(19) United States
(12) Patent Application Publication
(10) Pub. No.: US 2024/0173168 A1
(43) Pub. Date: May 30, 2024

(54) PROSTHETIC CAPSULAR DEVICES, SYSTEMS, AND METHODS

(71) Applicant: Omega Ophthalmics LLC, Versailles, KY (US)

(72) Inventors: Gary N. Wortz, Versailles, KY (US); Rick William Ifland, Versailles, KY (US); William Wiley, Chagrin Falls, OH (US)

(21) Appl. No.: 18/517,925

(22) Filed: Nov. 22, 2023

Related U.S. Application Data
(60) Provisional application No. 63/428,102, filed on Nov. 27, 2022.

(51) Int. Cl. A61F 9/00; A61F 2/16

(52) U.S. Cl. CPC A61F 9/0017; A61F 2/16; A61F 2002/16901; A61F 2230/0065; A61F 2250/0067

(57) ABSTRACT

The embodiments herein generally relate to systems, methods, and devices, including prosthetic capsular devices configured to be inserted in a natural capsular bag of an eye, the prosthetic capsular device comprising: a housing structure comprising an anterior portion and a posterior portion, an interior cavity, a groove formed along the circumference of the interior cavity, and a drug delivery device having a ring or an arc configuration, wherein the drug delivery device is held in place within the groove within the interior cavity of the housing structure.

### INCORPORATION BY REFERENCE TO ANY PRIORITY APPLICATIONS

The present application claims the benefit under 35 U.S.C. § 119(c) of U.S. Provisional Patent Application No. 63/428,102, filed Nov. 27, 2022, which is incorporated herein by reference in its entirety under 37 C.F.R. § 1.57. Any and all applications for which a foreign or domestic priority claim is identified in the Application Data Sheet as filed with the present application are hereby incorporated by reference under 37 CFR 1.57.

### BACKGROUND

The embodiments of the disclosure generally relate to ophthalmic prosthetics, and more particularly to devices, systems, and methods for a prosthetic apparatus for insertion into an eye.

### DESCRIPTION

Cataract surgery is one of the most successfully and most frequently performed surgical procedures in the United States. Each year, millions of people achieve a dramatic improvement in their visual function thanks to this procedure. With the increasing proportion of the U.S. population reaching their retirement years, there is expected to be an almost doubling of the demand for cataract surgery over the next twenty years from 3.3 million to over 6 million annually. In response to the increased demand, more ophthalmologists may be trained and certified to perform cataract surgery, and each trained and certified ophthalmologist may perform more cataract surgeries each year.

### SUMMARY

For purposes of this summary, certain aspects, advantages, and novel features of the invention are described herein. It is to be understood that not all such advantages necessarily may be achieved in accordance with any particular embodiment of the invention. Thus, for example, those skilled in the art will recognize that the invention may be embodied or conducted in a manner that achieves one advantage or group of advantages as taught herein without necessarily achieving other advantages as may be taught or suggested herein.

Some embodiments herein are directed to a prosthetic capsular device configured to be inserted in a natural capsular bag of an eye, the prosthetic capsular device comprising: a housing structure comprising: an anterior portion comprising: an anterior circular opening; an anterior rim surrounding the anterior circular opening and defining a perimeter of the anterior circular opening; and an anterior sidewall connected to the anterior rim and extending laterally outward and posteriorly from the anterior rim to a center plane of the housing structure; a posterior portion comprising: a posterior circular opening; a posterior rim surrounding the posterior circular opening and defining a perimeter of the posterior circular opening; and a posterior sidewall connected to the posterior rim and extending laterally outward and anteriorly from the posterior rim to the longitudinal center plane of the housing structure; an interior cavity formed between the anterior circular opening and the posterior circular opening, the interior cavity configured to house a drug delivery device; and a groove formed by one or more ribs, the one or more ribs formed along a circumference of the interior cavity at the longitudinal center plane of the housing structure, wherein each rib of the one or more ribs comprises a top surface and a bottom surface formed a rib angle, and wherein the groove is configured to hold the drug delivery device in place within the interior cavity of the housing structure; and a drug delivery device having a ring or an arc configuration, wherein the drug delivery device is held in place within the groove formed by the one or more ribs within the interior cavity of the housing structure.

In some embodiments, the drug delivery device, when viewed in profile, comprises a circle, a square, a rectangle, an oval, a triangle, a rounded triangle, a pentagon, a trapezoid, an ellipse, a semicircle, a hexagon, an octagon, a ring, a rhombus, or a star shape. In some embodiments, the drug delivery device comprises a length and a width, and wherein the ration of the length to the width is between about 2:1 and about 200:1. In some embodiments, the length is between 1 mm and 50 mm.

In some embodiments, the drug delivery device contacts an interior surface or an interior sidewall of the housing structure. In some embodiments, the drug delivery device is configured to flex or bend from a straight configuration to the ring or the arc configuration.

In some embodiments, the drug delivery device comprises an outer diameter between about 3 mm and about 12 mm. In some embodiments, a length of the drug delivery device is substantially equal to a circumference of the interior cavity of the housing structure. In some embodiments, a length of the drug delivery device is greater than a circumference of the interior cavity of the housing structure, such that at least a portion of the drug delivery device overlaps when the drug delivery device is held in the interior cavity.

In some embodiments, a length of the drug delivery device is less than a circumference of the interior cavity of the housing structure. In some embodiments, the prosthetic capsular device further comprises a second drug delivery device having an arc configuration, wherein the second drug delivery device is held in place within the groove formed by the one or more ribs within the interior cavity of the housing structure. In some embodiments, the second drug delivery device comprises a second length, wherein a sum of the length of the drug delivery device and the second length of the second drug delivery device is about equal to the circumference of the interior cavity of the housing structure. In some embodiments, the drug delivery device and the second drug delivery device substantially form an entire ring shape within the groove of the housing structure.

In some embodiments, the drug delivery device comprises a bio-erodible material. In some embodiments, the bio-erodible material comprises collagen, alginates, poly(lactic-glycolic acid) (PLGA), polyanhydrides, poly caprolactone, poly(trimethylene carbonate), or polyphosphazenes. In some embodiments, the bio-erodible material is impregnated or infused with one or more drugs. In some embodiments, the one or more drugs comprise anti-inflammatory drugs (NSAIDs, Steroids), antifibrotics (Mitomycin), immune modulators (Cyclosporin, Tacrolimus, methotrexate), Anti-viral (Gancyclovir, Valcyclovir, acyclovir), Antibiotics, Antifungal (Voriconazol, Natamycin, Amphoteracin B), or Anti-Vegf drugs (Avastin, Lucentis, Eylea).

In some embodiments, the drug delivery device comprises silicone, vulcanized silicone, and/or a silicone polymer. In some embodiments, the drug delivery device comprises one or more holes, eyelets, slits, and/or openings. In some embodiments, the one or more holes, eyelets, slits, and/or openings are configured to allow the drug delivery device to be placed within the interior cavity of the housing structure with an injector.

### BRIEF DESCRIPTION OF THE DRAWINGS

The drawings are provided to illustrate example embodiments and are not intended to limit the scope of the disclosure. A better understanding of the systems and methods described herein will be appreciated upon reference to the following description in conjunction with the accompanying drawings, wherein:

FIG. 1 illustrates an example prosthetic capsular device according to some embodiments herein.

FIG. 2 is an anterior side perspective view of the example prosthetic capsular device of FIG. 1.

FIG. 3 illustrates another example prosthetic capsular device according to some embodiments herein.

FIG. 4 is an anterior side perspective view of the example prosthetic capsular device of FIG. 3.

FIG. 5 illustrates another example prosthetic capsular device according to some embodiments herein.

FIG. 6 illustrates another example prosthetic capsular device according to some embodiments herein.

FIG. 7 illustrates another example prosthetic capsular device according to some embodiments herein.

FIG. 8 illustrates another example prosthetic capsular device according to some embodiments herein.

FIG. 9 illustrates another example prosthetic capsular device according to some embodiments herein.

FIG. 10 illustrates another example prosthetic capsular device according to some embodiments herein.

FIG. 11 illustrates another example prosthetic capsular device according to some embodiments herein.

FIG. 12 illustrates another example prosthetic capsular device according to some embodiments herein.

FIG. 13 illustrates another example prosthetic capsular device according to some embodiments herein.

FIG. 14 illustrates another example prosthetic capsular device according to some embodiments herein.

FIG. 15 illustrates another example prosthetic capsular device according to some embodiments herein.

FIG. 16 illustrates another example prosthetic capsular device according to some embodiments herein.

FIG. 17 illustrates another example prosthetic capsular device according to some embodiments herein.

FIG. 18 illustrates another example prosthetic capsular device according to some embodiments herein.

FIG. 19 illustrates another example prosthetic capsular device according to some embodiments herein.

FIG. 20 illustrates another example prosthetic capsular device according to some embodiments herein.

FIG. 21 illustrates another example prosthetic capsular device according to some embodiments herein.

FIG. 22 illustrates another example prosthetic capsular device according to some embodiments herein.

FIG. 23 illustrates another example prosthetic capsular device according to some embodiments herein.

FIG. 24 illustrates another example prosthetic capsular device according to some embodiments herein.

FIG. 25 illustrates another example prosthetic capsular device according to some embodiments herein.

FIG. 26 illustrates another example prosthetic capsular device according to some embodiments herein.

FIG. 27 illustrates another example prosthetic capsular device according to some embodiments herein.

FIG. 28 illustrates another example prosthetic capsular device according to some embodiments herein.

FIG. 29 illustrates another example prosthetic capsular device according to some embodiments herein.

FIG. 30 illustrates another example prosthetic capsular device according to some embodiments herein.

FIG. 31 Illustrates an example diagram of the interaction between a prosthetic capsular device and an iris of the eye.

FIG. 32 illustrates an example diagram of a prosthetic capsular device within an eye.

FIG. 33 illustrates an example image of a prosthetic capsular device within an eye according to some embodiments herein.

FIG. 34 illustrates another example prosthetic device according to some embodiments herein.

FIG. 35 illustrates another example prosthetic device according to some embodiments herein.

FIG. 36 illustrates another example prosthetic device and an example intraocular lens therein according to some embodiments herein.

FIG. 37 illustrates another example prosthetic device and an example intraocular lens therein according to some embodiments herein.

FIG. 38 illustrates another example prosthetic device and an example intraocular lens therein according to some embodiments herein.

FIG. 39 illustrates another example prosthetic device and an example intraocular lens therein according to some embodiments herein.

FIG. 40 illustrates an example injector cartridge for use in a method of inserting a prosthetic intraocular device and/or an intraocular lens according to some embodiments herein.

FIG. 41 illustrates another example prosthetic device according to some embodiments herein.

FIG. 42A illustrates a schematic of a kit for use in an ophthalmic surgical procedure according to some embodiments herein.

FIGS. 42B-42G illustrate schematics of a drug delivery device according to some embodiments herein.

FIG. 42H illustrates a drug delivery device in an example prosthetic device according to some embodiments herein.

FIGS. 43A-43B illustrate a flow chart of methods for inserting a kit according to some embodiments herein.

FIGS. 44A-44D illustrate another example prosthetic device according to some embodiments herein.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be conducted in a manner that achieves or optimizes one advantage or group of advantages as taught herein without necessarily achieving other aspects or advantages as may also be taught or suggested herein.

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present technology.

Devices and methods that help provide the desired refractive endpoint in cataract surgery are described in U.S. Pat. Nos. 8,900,300, 9,414,907, 9,358,103, and 10,603,162, each of which is hereby incorporated by reference in its entirety. All patents, patent applications, and other documents referred to in this application are incorporated by reference herein in their entirety.

In addition to the increase in demand for cataract surgery, technological advances have increased patient expectations for the surgery. The procedure takes a short amount of time to perform, and patients expect quick recovery of visual function. Patients are also asking their ophthalmologist to give them the restoration of more youthful vision without glasses through the use multifocal intraocular lenses, extended depth of focus lenses, accommodating lenses, other presbyopia correcting lenses, toric lenses, and monovision, to name a few. Despite accurate preoperative measurements and excellent surgical technique, post-surgical outcomes may vary due to undesirable physiological interaction with surgical implants.

Some embodiments herein are directed to prosthetic capsular devices and methods that address problems associated with prior devices. For example, the prosthetic capsular devices herein may be designed to eliminate, reduce, or mitigate contact between the iris pigment epithelium of an eye with the prosthetic capsular device. Implantation of an intraocular lens (IOL) or previous prosthetic capsular devices has become a routine practice among many surgeons, and several studies describe advantages of fixation of IOLs and other devices within the natural capsular bag. However, posterior iris chafing by the devices and IOLs may cause pigment dispersion and related inflammatory complications, specifically, the well described Uveitis-Glaucoma-Hyphema (UGH) syndrome. For example, the chafing may cause blurred vision, ocular pain, and headaches, pigmentary dispersion within the eye and on the IOL surface, iris trans illumination defects, iris changes including vacuolization/disruption/loss of the pigmented layer, iris thinning and iris atrophy, among others. With increased pigment shedding from the iris, the eye may experience iris transillumination defects, deposition of pigment of the corneal endothelium (Krukenbergs spindle), and angle pigmentation along the trabecular meshwork. Without being limited by any particular theory, it is postulated that the size and shape, including relatively large anterior-posterior thicknesses and sharp or straight edges, of previous prosthetic devices caused or contributed to this chafing and related complications. Thus, the prosthetic devices described herein are designed to eliminate, reduce, or mitigate posterior iris chafing.

Another problem associated with previous IOLs and prosthetic devices with implanted IOLs is lens tilt. Lens tilt occurs when the angle between the optical axis and the visual axis of the IOL are not colinear, which may occur if the IOL becomes misplaced within the natural capsular bag and/or the prosthetic capsular device. Lens tilt, along with decentration of the IOL, may cause suboptimal refractive outcomes for patients. For example, lens tilt may result in astigmatism and higher order aberrations. Substantial amounts of IOL tilt or decentration may cause enough astigmatism to significantly affect quality of vision. Multifocal, toric, and toric multi-focal are more sensitive to slight changes in tilt compared to monofocal IOLs and centration parameters need to be particularly accurate and precise. Thus, the prosthetic capsular devices described herein may be configured to secure IOLs therein such that the possibility of lens tilt and/or decentration is minimized.

The intravitreal space has been utilized widely over the past 30 plus years for injection of steroids for treatment of inflammatory and vascular disease processes. In the past 15 years or so, the intravitreal space has been widely utilized for treatment of wet age-related macular degeneration and other vascular proliferative retinal diseases with injection of Anti-Vegf monoclonal antibodies, dexamethasone, and fluocinolone acetanide and other drugs.

Drug delivery in the vitreous cavity advantageously allows for a large potential volume of drugs to be placed in close proximity to the retina. However, drug delivery in the vitreous cavity requires an injection, usually monthly or some other period. The medications are sometimes opaque creating a barrier to light being received by the retina and/or loss of vision. Intravitreal injections can damage the natural lens capsule and lens, increase intraocular pressure, and/or can cause retinal detachment and endophthalmitis or other damage to the eye tissue.

The anterior segment of the eye has only recently been regarded as a possible target for drug delivery. Recently drugs such as dexamethasone and bimatorprost have been FDA approved for the treatment of post-operative inflammation and glaucoma, respectively. While both of these treatment options can be effective, each is plagued by having poor control of drug location. When a drug is placed in the anterior segment it can cause damage if it migrates adjacent to the corneal endothelium. Similarly, iris atrophy has been noted in patients with certain drugs being in prolonged contact with the iris. Unlike the posterior segment (vitreous cavity), controlling the location of controlled release drug implants plays a significant role in preventing complications.

Accordingly, it can be advantageous to utilize an ophthalmic device configured to continuously deliver drugs to the eye tissue thereby eliminating the need for periodic injections into the eye and avoiding the negative consequences of such injections. In some embodiments, such an ophthalmic device can comprise a prosthetic capsular device for insertion into the natural capsular bag, and in some embodiments, the prosthetic capsular device can be configured to create a space or void or volume within the eye for receiving drug delivery devices for placement in the eye. In some embodiments, an IOL is first inserted into the natural capsular bag followed by the insertion of the prosthetic capsular device. In some embodiments, the prosthetic capsular device is positioned anterior to an IOL such that the prosthetic capsular device can create an extant intracapsular space accessible through the pupil and capsulorhexis. A drug delivery device can be inserted into the prosthetic capsular device, and in some embodiments be positioned peripherally to the visual pathway such that the drug delivery device does not or does not materially or does not substantially obstruct, interfere, or block the visual pathway. In some environments, the drug delivery device is configured to dissolve or partially dissolve, or otherwise change over time, thereby releasing into the eye at least a portion of the drugs in the drug delivery device. In some embodiment, the prosthetic capsular device may receive one or more secondary drug delivery devices, for example, after a first drug delivery device dissolves or after substantially all the drugs in the drug delivery device has been released into the eye, or for example, simultaneous or subsequent with the first drug delivery device in order to release multiple drugs into the eye at the same time.

In some embodiments, a drug or drug delivery device can be inserted into the housing of the device, such that the prosthetic capsule is impregnated with an active pharmaceutical agent during the molding process. In some embodiments, the prosthetic capsule can be coated either on the interior, the exterior, or both with a drug after the molding process such that the drug dissolves off of the surface of the prosthetic capsule. In some embodiments, the prosthetic capsule is placed into a solution containing active pharmaceutical agents at some point after molding (either before packaging, or afterward such as immediately prior to surgery) such that it soaks up drug into the silicone pores, through which drug can dissolve into the eye over time. In some embodiments, the drug delivery device is composed of a biocompatible flexible non-absorbable tubular material with active pharmaceutical agent inside of it, with an opening or multiple openings allowing for the controlled release of the drug. In this embodiment, the drug containing tube could be explanted from the eye once the active pharmaceutical agent has substantially dissolved and a new device could be inserted. This process could take place over the patient's lifetime for extant treatment of disease processes because the housing of the prosthetic capsular device is configured to allow for the insertion and removal of the drug delivery device or allow for the insertion of new drugs. In some embodiments, the housing of the prosthetic capsular device is configured for the insertion of new drug delivery devices or drugs over years and decades because the housing of the prosthetic capsular device is configured to remain in a self-expanded state while positioned in the natural capsular bag, and the housing of the device comprises an opening that is configured to remain open while positioned in the natural capsular bag, and the opening allows for drug delivery devices and/or drugs to be inserted into the interior of the housing of the prosthetic capsular device. In some embodiments, the drug delivery device can be bio erodible. In some embodiments, the drug delivery device may release one or more drug into the natural capsular bag. In some embodiments, the drug delivery device can be configured to comprise a substantially linear elongated shape configuration. In some embodiments, the substantially linear elongated shape configuration enables the drug delivery device to be easily inserted into a delivery cannula or trocar or injector in order to position the drug delivery device within the prosthetic capsular device. In some embodiments, the substantially linear elongated shaped drug delivery device is configured to bend within the prosthetic capsular device. In some embodiments, the bending of the linear elongated shaped drug delivery device allows the drug delivery device to be positioned in the peripheral area of the prosthetic capsular device in order to avoid, obstruct or otherwise block the visual pathway of the eye. In some embodiments, the substantially linear elongated shaped drug delivery device can comprise end portions that are substantially curved whereas a mid-portion of the drug delivery device is substantially linear.

In some embodiments, the drug delivery device can be configured to comprise a substantially curved shaped configuration. In some embodiments, the drug delivery device can be configured to comprise a bias such that the drug delivery device takes on a curved shaped configuration. In some embodiments, the curved shaped drug delivery device can be straightened into a more linear configuration in order for the drug delivery device to be inserted through an insertion cannula or trocar or injector for implantation into the prosthetic capsular device. In some embodiments, after the curved shaped drug delivery device is positioned into the prosthetic capsular device, the inherent bias of the drug delivery device allows the drug delivery device to take on a curved shape within the prosthetic capsular device. In some embodiments, the bias of the curved shaped drug delivery device enables the drug delivery device to position itself into the peripheral area of the prosthetic capsular device in order to avoid or interfere or obstruct with the visual pathway of the eye. In some embodiments, the biased drug delivery device is configured to self-expand within the prosthetic capsular device in such a way as to take on a curved shape. In some embodiments, the drug delivery device can bend and/or self-expand from a substantially straight configuration to a substantially ring-shaped configuration. In some embodiments, the drug delivery device can comprise a substantially cylindrical shape. In some embodiments, the drug delivery device can comprise a profile shape. In some embodiments, the profile shape can be substantially circular shaped, oval shaped, elliptical shaped, diamond shaped, triangle shaped, rectangle shaped, pentagon shaped, hexagon shaped, octagon shaped, and/or any other shape. In some embodiments, the drug delivery device comprises substantially smooth and/or rounded edges, and in some embodiments, the smooth and/or rounded edges prevent scraping against the side walls of the prosthetic capsular device. In some embodiments, the prosthetic capsular device comprises a groove portion in the interior of the housing of the prosthetic capsular device, wherein the groove portion is configured to receive and/or retain the drug delivery device once the drug delivery device has been inserted into the prosthetic capsular device and self-expands and/or returns to drug delivery device's biased curved and/or ring-shaped configuration.

Additional technology can be coupled to the prosthetic capsular device. In some embodiments, the prosthetic capsular device can be coupled to the ciliary muscle so the ciliary muscles can expand and/or contract the prosthetic capsular device. In some embodiments, one or more lenses coupled to the prosthetic capsular such that the ciliary muscle can alter or control a distance between the one or more lenses allowing for improved vision.

In some embodiments, the device comprises one or more fixation members extending anteriorly from an exterior surface of the device. In some embodiments, one or more fixation members are positioned on an anterior surface of the device. In some embodiments, one or more fixation members are positioned on a side wall surface of the device. In some embodiments, the one or more fixation members are positioned on a posterior surface of the device. In some embodiments, the one or more fixation members are positioned on a surface between an anterior surface and a side wall surface of the device. In some embodiments, the one or more fixation members are positioned on a surface between a posterior surface and a side wall surface of the device. In some embodiments, the one or more fixation members comprise one or more protrusions that extend outwardly from the exterior surface of the device. In some embodiments, the one or protrusions comprise rounded surfaces to avoid causing irritation and/or inflammation and/or other damage to the tissue of the eye, for example, the interior surface of the natural capsular bag. In some embodiments, the one or more protrusions comprise one or more openings for receiving sutures that enable fixation of the one or more protrusions to tissues of the eye, for example, the ciliary muscle. In some embodiments, one or more fixation members can be inserted into an area of the eye, for example, the sulcus of the eye. In some embodiments, one or more fixation members can be inserted through the anterior capsule of the eye and into the sulcus of the eye. In some embodiments, the one or more fixation members can be inserted into the sulcus by creating one or more microrhexises in the anterior capsule of the eye, wherein the created microrhexises can be configured to receive the one or more fixation members of the device in order to secure the device to the eye. In some embodiments, the ciliary muscle can move the one or more fixation members causing the device to expand and/or contract. In some embodiments, the expansion and/or contraction can cause the device to expand and/or contract laterally between the side walls of the eye, and in some embodiments, the lateral expansion and/or contraction of the device can cause the device to expand and/or contract in an anterior and/or posterior direction, for example, the profile of the device can become more circular and/or elliptical. In some embodiments, the expansion and/or contraction of the device can allow for pressure to be applied to other devices, for example, an IOL, positioned within and/or on the device, thereby in some embodiments causing the characteristics of the other devices to change, for example, changing the magnification and/or focus of the IOL.

In some embodiments, a posterior lens and/or an anterior lens can be inserted into and/or onto the device. In some embodiments, the posterior lens can be coupled or positioned adjacent to a posterior surface of the device. In some embodiments, the posterior lens is positioned adjacent to an interior surface or an exterior surface of the posterior portion of the device. In some embodiments, the posterior lens is inserted into the eye prior to the device being inserted into the eye, or simultaneously with the device, and in some embodiments, the posterior lens is kept in position by compression forces exerted on the posterior lens by the outward expansion of the device and/or by the natural capsular bag conforming around the posterior lens and device. In some embodiments, the anterior lens can be coupled or positioned adjacent to an anterior surface of the device. In some embodiments, the anterior lens is positioned adjacent to an interior surface or an exterior surface of the anterior portion of the device. In some embodiments, the anterior lens is inserted into the eye after the device is inserted into the eye, or simultaneously with the device, and in some embodiments, the anterior lens is kept in position by compression forces exerted on the anterior lens by the outward expansion of the device and/or by the natural capsular bag conforming around the anterior lens and device similar to a sock-like member. In some embodiments, the posterior lens can be coupled to or inserted in a posterior opening of the device. In some embodiments, the anterior lens can be coupled to or inserted in an anterior opening of the device. In some embodiments, expansion and/or contraction of the device can change a distance between the posterior lens and the anterior lens, thereby a magnification and/or power and/or focus of the light entering into the eye through the anterior and/or posterior lens.

In some embodiments, the prosthetic capsular device is configured to receive an interior lens within the interior of the housing of the prosthetic capsular device. In some embodiments, the prosthetic capsular device is configured to comprise one, and in some embodiments, the prosthetic capsular device is configured to comprise two or three lens simultaneously, for example, the prosthetic capsular device can be configured with one lens posterior to the prosthetic capsule device, one lens inside the interior of the prosthetic capsule device, and one lens positioned in the sulcus anteriorly to the prosthetic device. In some embodiments, one or more of the anterior lens and/or the interior lens and/or the posterior lens are configured to be light adjustable, for example, the exposure of light to the lens can cause the lens to darken to shield the eye from bright lights and/or UV rays and/or other electromagnetic radiation wavelengths.

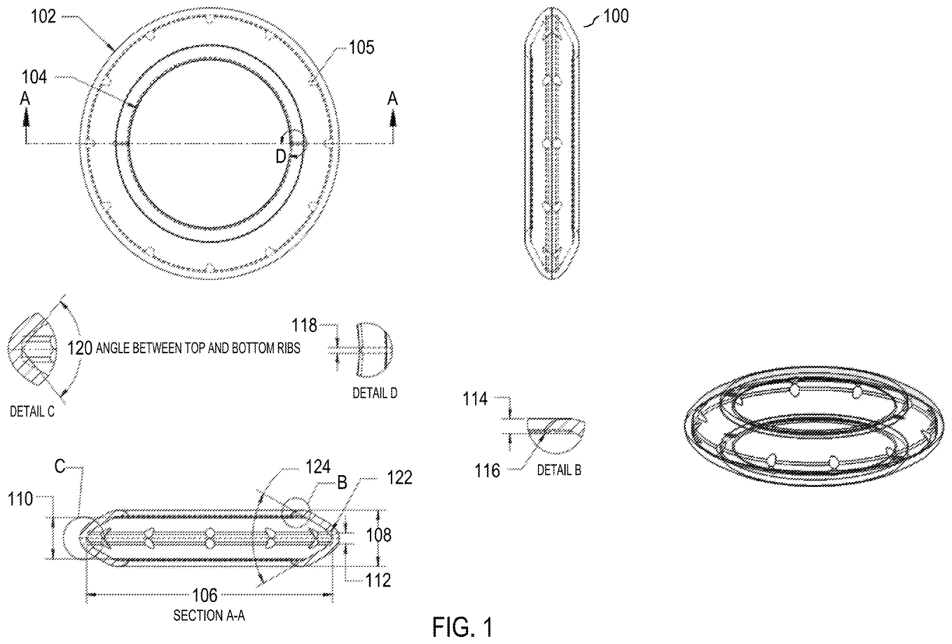

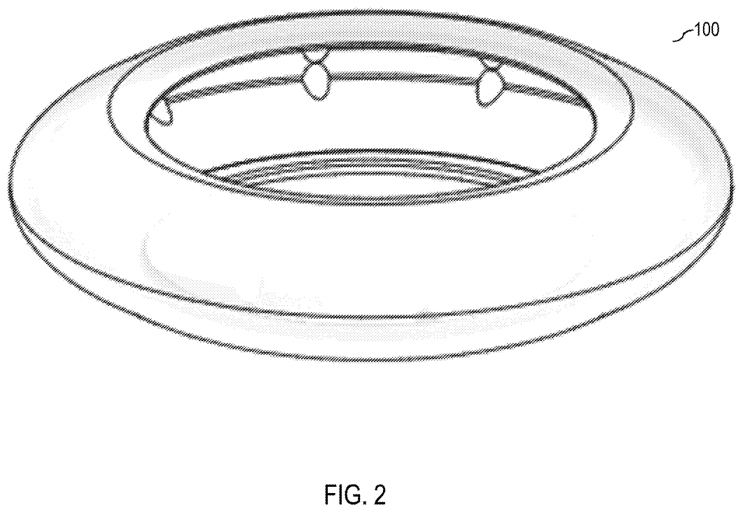

FIG. 1 illustrates various perspective views of an example of a prosthetic capsular device 100. In some embodiments, the device 100 includes features described with respect to the devices described in U.S. Pat. No. 10,603,162, which is hereby incorporated by reference in its entirety, or modifications thereof. FIG. 2 is an anterior side perspective view of the example prosthetic capsular device of FIG. 1.

In some embodiments, the device 100 includes features described with respect to the devices described in U.S. Pat. No. 9,358,103, which is hereby incorporated by reference in its entirety, or modifications thereof. For example, the device 100 can comprise an anterior side, a posterior side, and one or more sidewalls extending between the anterior side and the posterior side; a cavity or opening defined by the anterior side, posterior side, and the one or more sidewalls. Device 100 can be configured to comprise one or more intraocular lenses, electronic devices, or other intraocular devices held within the cavity. The IOLs may comprise any and all lens powers and designs that are currently known in the art of intraocular lenses, including, but not limited to: spherical, aspheric, wavefront, convex, concave, extended depth of focus, pinhole or small aperture, multifocal (diffractive, refractive, zonal), toric, accommodative, ultraviolet (UV) filtering, diffractive chromatic aberration reducing lenses, light adjustable lenses (ultraviolet light adjustable, femtosecond phase wrapping), and optical powers ranging from any positive diopter value (e.g., including +35 D and above) to any negative diopter value (e.g., including −35 D and below).

Further, in certain embodiments, device 100 includes one or more additional features. For example, device 100 can comprise a generally lenticular or lens-like shape as opposed to a box-like design. In other words, the generally shape of the device 100 can be more like the shape of a natural lens. Risks of negative and/or positive dysphotopsia can be reduced due to the generally lenticular shape of the device 100. Negative dysphotopsia is a widespread problem in cataract surgery, generally described by patients as a temporal dark crescent in their vision and is believed to occur either due to the optical phenomenon known as total internal reflection or by obstruction of light. This can occur either at the junction of the optic edge and the empty collapsed surrounding capsule forming a relatively planar surface, or due to the capsule overlapping a portion of the optic, most commonly the nasal aspect. In embodiments in which the implantable device 100 comprises an overall lens-like configuration, the capsule can be held open, preventing a relatively planar surface from being formed by fusion of the posterior and anterior capsule. More specifically, when light hits a curvilinear slice of the device 100, which can be made from silicone for example, it may travel through the curvilinear slice instead of bouncing off and causing a negative shadow as it generally would for flat surfaces. This may be especially true in the horizontal meridian across the 180-degree plane. As such, in some embodiments, device 100 does not comprise any flat edges or surfaces. In other words, every surface of the device 100 can be curvilinear. Flat optical surfaces can promote total internal reflection and are not found in the natural human lens or lens capsule in the native state. One goal of some of the embodiments described herein is to reduce negative dysphotopsias by not having any flat optical surfaces. Certain embodiments may have additional features such as an opaque or translucent tint. This may further enhance the reduction of positive dysphotopsias by blocking stray light that could be reflected off of the IOL border or haptic edges. This could also function as an artificial iris of sorts, depending on the color and opacity of the tint, blocking light that could be transmitted through an iris transillumination defect, a traumatically altered iris, or a surgical peripheral iridotomy, likewise preventing positive dysphotopsias and glare.

In some embodiments, substantially the whole device 100 can comprise silicone and/or a soft silicone polymer. In addition, in certain embodiments, substantially the whole device 100 can comprise a flexible and/or elastic material. As such, device 100 can be foldable or collapsible for implantation into the eye through a small incision. Once inserted into the eye, device 100 can naturally unfold and self-expand into its expanded configuration as illustrated in FIG. 1. Device 100 can comprise one or more capsular areas. The one or more capsular areas can be adapted to receive and/or hold an IOL. In some embodiments, one or more sidewalls can comprise a concave shape. For example, the interior surface of the one or more sidewalls can form a cavity. The cavity can be configured to hold an IOL, for example.

In some embodiments, device 100 comprises a single-molded design. In other words, the whole device 100, or substantially the whole device 100 can be molded from a single piece of material. For example, in some embodiments, substantially the whole device 100 can be molded of silicone using a silicone compression mold. In other embodiments, the device 100 or any portion thereof can be manufactured by 3D laser cutting, two photon lithography, additive manufacturing, 3D printing, compression molding, and/or any combination of the aforementioned manufacturing processes or others.

In some embodiments, the device 100 can be inserted through an incision between about 1.5 mm and about 3 mm (e.g., about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, ranges between such values, etc.).

Further, in some embodiments, a length of a major axis of the device 100 or a length measured from the outermost end of one sidewall to the outermost end of another sidewall along a major axis of the device 100 can be about 9.65 mm. In other embodiments, the length of the major axis of the device 100 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 100 may comprise a diameter 102 of the device 100.

In some embodiments, the thickness of silicone or other material of the device 100 can be about 0.25 mm. In certain embodiments, the thickness of silicone or other material of the device 100 can be about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, and/or within a range defined by two of the aforementioned values.

In some embodiments, the thickness of the silicone or other material of the device 100 varies depending on the portion of the device 100. In other words, some portions of device 100 can be made of thinner materials while other portions of device 100 can be made of thicker materials. For example, certain portions of the device that provide support to the anterior portion of the device 100 may be made with thicker materials for added support.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 104, which can be a diameter of an anterior opening and/or a posterior opening.

In some embodiments, the shape and size of the device 100 may minimize anterior, posterior, and/or radial protrusion into the natural capsular bag relative to previously used capsular devices. In some embodiments, the device 100 may be smaller in certain dimensions especially towards the anterior and periphery of the device. In some embodiments, sharp and/or straight edges or sides may not be present in the device 100 to reduce friction between the device 100 and the posterior aspect of the iris of an eye. In some embodiments, the smaller size, decrease in anterior, posterior, and/or radial protrusion into the natural capsular bag, and smoothened or curved edges, may result in the device 100 having an enhanced biocompatibility profile and/or may reduce inflammation caused by the device in the eye. In some embodiments, the unique shape and design of device 100 may result in a decrease and/or elimination of inflammation of the eye (e.g., anterior of the eye) upon insertion of the device. In some embodiments, the decrease and/or elimination of post-insertion inflammation resulting from the shape of the device 100 may result in a decrease and/or elimination of the need for post-operative anti-inflammatory medications such as, e.g., steroids or nonsteroidal anti-inflammatory drugs (NSAIDs). It may also result in a decrease and/or elimination of device removals and/or replacements, which may be needed if inflammation cannot be reduced or removed.

The device 100 can be self-expandable to keep the capsule open. The device 100 can comprise at least three different planes. For example, a first plane can correspond with the posterior opening or end of the device, where an IOL can be attached. A second plane can correspond with the anterior opening or end of the device, where another refractive surface or IOL can be attached. In some embodiments, device 100 comprises a symmetrical device such that the anterior opening and posterior opening are determined by the device 100 position in the eye. A third plane can be positioned in between the posterior end and the anterior end, for example, along a ridge formed in the central portion, where another an IOL can be attached. In some embodiments, the central portion may comprise a continuous lateral portion interposed between the anterior portion and the posterior portion. In some embodiments, the continuous lateral portion protrudes radially beyond the anterior portion and the posterior portion. In some embodiments, the continuous lateral portion fully encloses a lateral side of the housing structure, wherein an internal cavity of the continuous lateral portion forms a groove for containing an IOL. In some embodiments, the central portion may comprise a continuous lateral portion comprising an exterior surface comprising a rounded bulge, the rounded bulge extending radially beyond the anterior portion and the posterior portion. In some embodiments, the continuous lateral portion comprises an interior surface comprising a groove or ridge, wherein at least a portion of the interior surface is formed at an acute angle or an obtuse angle relative to the anterior portion and the posterior portion.

In some embodiments, the prosthetic capsular devices comprise one or more orientation designation indicators or mechanisms 118 configured to serve as a marker to indicate the direction and/or orientation of the prosthetic device before, during, and/or after insertion into the eye. In some embodiments, the one or more orientation designation mechanisms 118 may be located on the anterior side, the posterior side, and/or on the interior and/or exterior sidewalls of the prosthetic capsular device. In some embodiments, the one or more orientation designation mechanisms 118 may assist and/or allow a surgeon or medical professional to determine or perceive if the prosthetic capsular device is oriented correctly before, during, and/or after insertion into the eye.

In some embodiments, the one or more orientation indicators may comprise visual distinguishing factors on the anterior side, the posterior side, and/or on the interior and/or exterior sidewalls of the prosthetic capsular device. For example, the anterior side, the posterior side, and/or the interior and/or exterior sidewalls may differ based on varying structural features, axis marks, colors, shapes, textures, tones, shades, brightness, outlines, sizes, text indicators, engravings, and icons, among others. In some embodiments, the one or more orientation designation indicators facilitate the current orientation of the prosthetic capsular device before, during, and after insertion into the eye and serve as measurement tools to measure, for example, rotational stability.

In some embodiments, the one or more orientation designation indicators comprise a protuberance, nub, protrusion, projection, bulge, or other structure extending from a surface of the housing 100. In some embodiments, the one or more orientation designation indicators comprise a visual marker such as a hole or aperture. In some embodiments, the visual marker may serve as a reference point to measure rotational stability and position of the prosthetic capsular device 100 before, during, and/or after insertion into the eye. In some embodiments, the one or more orientation designation indicators 118 may extend radially inward from the diameter 104 of the anterior opening and/or the posterior opening. However, in some embodiments, the one or more orientation designation indicators may extend radially inward or radially outward from any structure of the prosthetic device 100 and/or an IOL coupled to the device. In some embodiments, it may be preferable for the one or more orientation designation indicators to extend radially inwardly from the anterior opening to provide optimal visibility to a surgeon and/or medical professional and to avoid unnatural exterior protrusions into the natural capsular bag.

In some embodiments, the prosthetic capsular device 100 may comprise about 2 orientation designation indicators. In some embodiments, the number of orientation designation indicators 118 may be about 1, 2, 3, 4, 5, 6, 7, 8, 9, 10, 11, 12, 13, 14, 15, 20, 25, 30, 35, 40, 45, 50, 100, and/or within a range defined by two of the aforementioned values. An example orientation designation indicator 118 is illustrated in Detail D of FIG. 1. In some embodiments, the orientation designation indicator 118 may comprise a thickness of about 0.10 mm. In some embodiments the orientation designation indicator 118 may comprise a thickness of between about 0.01 mm and 0.30 mm.

In some embodiments, a prosthetic capsular device configured to be inserted in a natural capsular bag of an eye after removal of a lens can comprise a housing structure 100 capable of containing one or more intraocular devices and/or refractive surfaces. In particular, the housing structure can comprise an anterior side, wherein the anterior side comprises an anterior opening that can be elliptical, circular, arcuate, triangular, rectangular, polygonal, or otherwise shaped as shown in the provided Figures, wherein the anterior opening is capable of allowing at least one of insertion, removal, or replacement of an intraocular lens device, and wherein the anterior opening is further configured to be coupled to a lens to cover the anterior opening; a posterior side, wherein the posterior side comprises an posterior opening that can be elliptical, circular, arcuate, triangular, rectangular, polygonal, or otherwise shaped as shown in the provided Figures, wherein the posterior opening is capable of allowing at least one of insertion, removal, or replacement of an intraocular lens or device, and wherein the posterior opening is further configured to be coupled to an intraocular lens or device to cover the posterior opening; and a continuous lateral portion interposed between the anterior portion and the posterior portion, wherein the continuous lateral portion protrudes radially beyond the anterior portion and the posterior portion, wherein the continuous lateral portion fully encloses a lateral side of the housing structure, wherein an internal cavity of the continuous lateral portion forms a groove or ridge for containing an intraocular lens or device within, for example, an anterior portion of the device. The continuous lateral portion may not have any openings, for example along the lateral portion of the device in some embodiments. The housing structure 100 can be symmetrical over a plane at a midpoint of the continuous lateral portion between the anterior portion and the posterior portion.

In some embodiments, the ridge or groove may comprise one or more ribs 105. The ribs 105 are shown in detail along the ridge as Detail C in FIG. 1. In some embodiments, the ribs may be configured to hold an intraocular lens within device 100. For example, the ribs 105 may be configured to reduce mobility of an intraocular lens within device 100, such that lens tilt, lens rotation, and/or decentration is reduced or eliminated. In some embodiments, the ribs 105 may comprise a top surface and a bottom surface with a rib angle 120 between the top and bottom surface. In some embodiments, the rib angle may comprise about 100°. In some embodiments, the rib angle may comprise about 10° to about 180°. For example, in some embodiments, the rib angle may be about 10°, about 15°, about 20°, about 25°, about 30°, about 35°, about 40°, about 45°, about 50°, about 55°, about 60°, about 65°, about 70°, about 75°, about 80°, about 85°, about 90°, about 95°, about 100°, about 105°, about 110°, about 115°, about 120°, about 125°, about 130°, about 135°, about 140°, about 145°, about 150°, about 155°, about 160°, about 165°, about 170°, about 175°, about 180°, or any value between the aforementioned values. In some embodiments, the rib angle 105 may be determined based on the thickness 108 of the device 100, along with the diameter 102.

In some embodiments, device 100 may comprise about 12 ribs. In some embodiments, the device 100 may comprise between about 1 rib and 100 ribs. about 1 ribs, about 2 ribs, about 3 ribs, about 4 ribs, about 5 ribs, about 6 ribs, about 7 ribs, about 8 ribs, about 9 ribs, about 10 ribs, about 11 ribs, about 12 ribs, about 13 ribs, about 14 ribs, about 15 ribs, about 16 ribs, about 17 ribs, about 18 ribs, about 19 ribs, about 20 ribs, about 21 ribs, about 22 ribs, about 23 ribs, about 24 ribs, about 25 ribs, about 26 ribs, about 27 ribs, about 28 ribs, about 29 ribs, about 30 ribs, about 31 ribs, about 32 ribs, about 33 ribs, about 34 ribs, about 35 ribs, about 36 ribs, about 37 ribs, about 38 ribs, about 39 ribs, about 40 ribs, about 41 ribs, about 42 ribs, about 43 ribs, about 44 ribs, about 45 ribs, about 46 ribs, about 47 ribs, about 48 ribs, about 49 ribs, about 50 ribs, about 51 ribs, about 52 ribs, about 53 ribs, about 54 ribs, about 55 ribs, about 56 ribs, about 57 ribs, about 58 ribs, about 59 ribs, about 60 ribs, about 61 ribs, about 62 ribs, about 63 ribs, about 64 ribs, about 65 ribs, about 66 ribs, about 67 ribs, about 68 ribs, about 69 ribs, about 70 ribs, about 71 ribs, about 72 ribs, about 73 ribs, about 74 ribs, about 75 ribs, about 76 ribs, about 77 ribs, about 78 ribs, about 79 ribs, about 80 ribs, about 81 ribs, about 82 ribs, about 83 ribs, about 84 ribs, about 85 ribs, about 86 ribs, about 87 ribs, about 88 ribs, about 89 ribs, about 90 ribs, about 91 ribs, about 92 ribs, about 93 ribs, about 94 ribs, about 95 ribs, about 96 ribs, about 97 ribs, about 98 ribs, about 99 ribs, or about 100 ribs.

In some embodiments, a thickness 108 of the device may comprise a maximum distance between the anterior side and posterior side of the device 100. In some embodiments, the thickness 108 of device 100 may be about 2.00 mm. In some embodiments, the thickness 108 of the device 100 may be about 1.50 mm. In some embodiments, the thickness 108 of the device 100 may be between about 0.5 mm and 4.0 mm. In some embodiments, the thickness 108 of the device 100 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

In some embodiments, device 100 may comprise a ridge thickness 112 comprising the size of the ridge locating ribs 105. In some embodiments, the ridge thickness 112 may be about 0.40 mm. In some embodiments, the ridge thickness 112 may be between about 0.10 mm and about 1.00 mm. In some embodiments, the ridge thickness may be configured to reduce the possibility of lens tilt by an intraocular lens located in the ridge and secured by ribs 105.

In some embodiments, device 100 may comprise an inner thickness 110 comprising a distance between an inner surface of the sidewall at the anterior opening and an inner surface of the sidewall at the posterior opening. In some embodiments, the inner thickness 110 may be about 1.48 mm.

In some embodiments, device 100 may comprise an inner diameter 106 comprising the distance between the interior surfaces of the sidewalls at the ridge. In some embodiments, the inner diameter 106 may be about 9.15 mm. In some embodiments, the interior diameter may be between about 5.00 mm and 15.00 mm.

In some embodiments, the sidewall at the anterior portion of the device 100 and the sidewall at the posterior of the device 100 may form a sidewall angle 124, formed at the ridge of the device 100. In some embodiments, the sidewall angle 124 may be about 34°. In some embodiments, the sidewall angle 124 may about 57°. In some embodiments, the sidewall angle 124 may be about 10° to about 180°. For example, in some embodiments, the sidewall angle 124 may be about 10°, about 15°, about 20°, about 25°, about 30°, about 35°, about 40°, about 45°, about 50°, about 55°, about 60°, about 65°, about 70°, about 75°, about 80°, about 85°, about 90°, about 95°, about 100°, about 105°, about 110°, about 115°, about 120°, about 125°, about 130°, about 135°, about 140°, about 145°, about 150°, about 155°, about 160°, about 165°, about 170°, about 175°, about 180°, or any value between the aforementioned values. In some embodiments, the sidewall angle 124 may be determined based on the thickness 108 of the device 100, along with the diameter 102.

In some embodiments, the cavity of the device may comprise a volume for maintaining the shape and size of the natural capsular bag. The volume may be formed by the angled sidewalls and may comprise a tapered confinement area, wherein the sidewall taper into the cavity at the anterior and posterior openings. In some embodiments, the taper may form a curve in the sidewall adjacent to the anterior and posterior openings. The curved section of the sidewall may provide a smoothed edge to reduce the impact of incidental contact with the posterior surface of the iris of the eye, or pressure transduced through the natural capsular bag and imparted onto the posterior surface of the iris of the eye. In some embodiments, the taper length 114 may comprise a distance between the exterior surface of the sidewall at its most anterior/posterior point and the exterior surface at the anterior/posterior opening. In some embodiments, the exterior surface may comprise curved surfaces 122 and 116 at the ridge and at the openings, respectively. The curved shape of the sidewalls may contribute to a reduction in post-surgical complications through minimization of contact or the severity of contact between the device 100 and the iris. The curved shape of the sidewall of the device 100 near the openings is shown in Detail B.

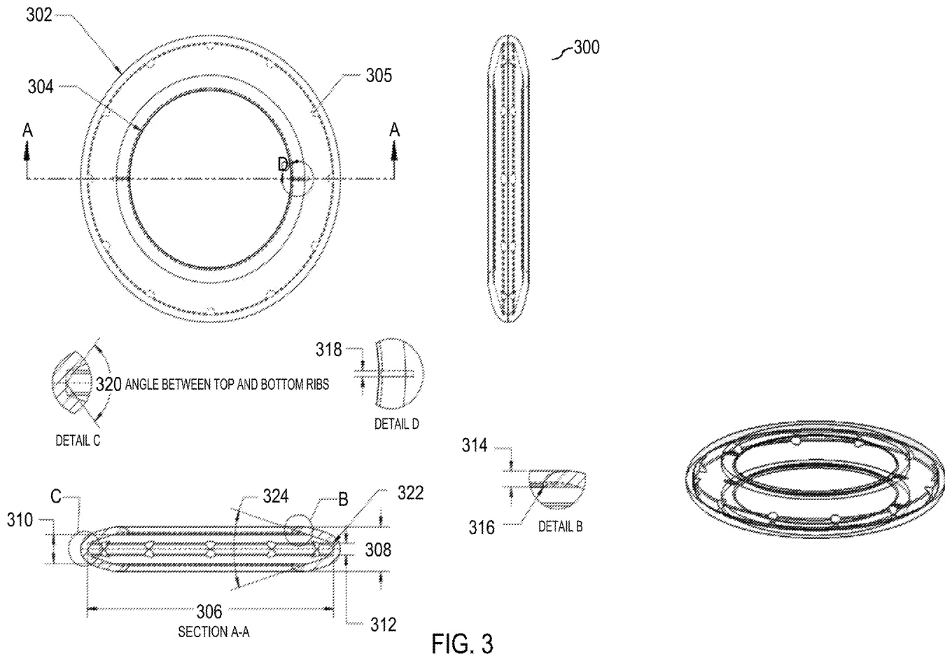

FIGS. 3-30 illustrate other example prosthetic capsular devices according to some embodiments herein. The embodiments of FIGS. 2-30 may comprise some or all of the features of devices described in U.S. Pat. No. 10,603,162 and of device 100. In addition, the embodiments of FIGS. 2-30 may comprise one or more additional features.

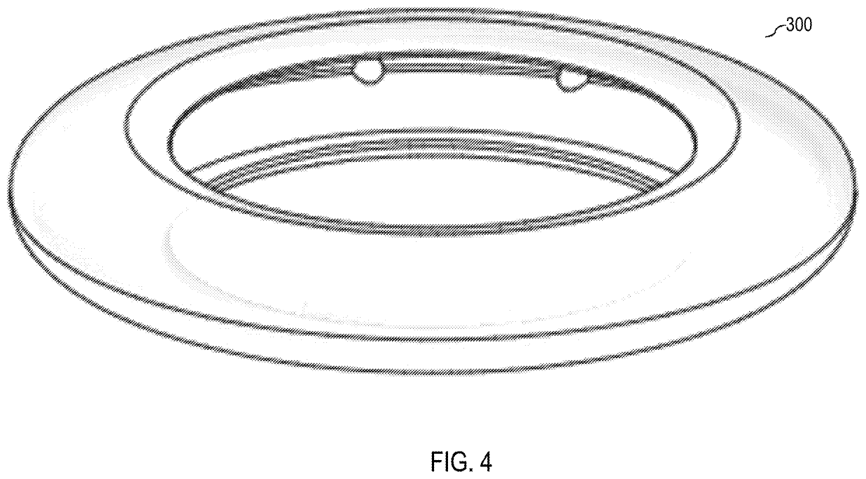

FIG. 3 illustrates another example prosthetic capsular device according to some embodiments herein. FIG. 4 is an anterior side perspective view of the example prosthetic capsular device of FIG. 3. In device 300 of FIGS. 3-4, the sidewall at the anterior portion of the device 300 and the sidewall at the posterior of the device 300 may form a sidewall angle 324, formed at the ridge of the device 300. In some embodiments, the sidewall angle 324 may be smaller than that of device 100. In some embodiments, this smaller sidewall angle may reduce the overall thickness and profile of the device 300 relative to device 100. As such, device 300 may be smaller and be used where a smaller profile is necessary depending on the needs of a patient.

In some embodiments, a length of a major axis of the device 300 or a length measured from the outermost end of one sidewall to the outermost end of another sidewall along a major axis of the device 300 can be about 9.65 mm. In other embodiments, the length of the major axis of the device 300 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 300 may comprise a diameter 302 of the device 300.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 304, which can be a diameter of an anterior opening and/or a posterior opening.

In some embodiments, the device 300 may comprise a ridge or groove comprising one or more ribs 305. The ribs 305 are shown in detail along the ridge as Detail C in FIG. 3. In some embodiments, the ribs may be configured to hold an intraocular lens within device 300. For example, the ribs 305 may be configured to reduce mobility of an intraocular lens within device 300, such that lens tilt, lens rotation, and/or decentration is reduced or eliminated. In some embodiments, the ribs 305 may comprise a top surface and a bottom surface with a rib angle 320 between the top and bottom surface. In some embodiments, the rib angle may comprise about 100°. In some embodiments, the rib angle may comprise about 10° to about 180°. For example, in some embodiments, the rib angle may be about 10°, about 15°, about 20°, about 25°, about 30°, about 35°, about 40°, about 45°, about 50°, about 55°, about 60°, about 65°, about 70°, about 75°, about 80°, about 85°, about 90°, about 95°, about 100°, about 105°, about 110°, about 115°, about 120°, about 125°, about 130°, about 135°, about 140°, about 145°, about 150°, about 155°, about 160°, about 165°, about 170°, about 175°, about 180°, or any value between the aforementioned values. In some embodiments, the rib angle 305 may be determined based on the thickness 308 of the device 300, along with the diameter 302.

In some embodiments, a thickness 308 of the device 300 may comprise a maximum distance between the anterior side and posterior side of the device 300. In some embodiments, the thickness 308 of device 308 may be about 2.00 mm. In some embodiments, the thickness 308 of the device 300 may be about 1.50 mm. In some embodiments, the thickness 308 of the device 300 may be between about 0.5 mm and 4.0 mm. In some embodiments, the thickness 308 of the device may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

In some embodiments, the prosthetic capsular device 300 comprises one or more orientation designation indicators or mechanisms 318 configured to serve as a marker to indicate the direction and/or orientation of the prosthetic device before, during, and/or after insertion into the eye. In some embodiments, the one or more orientation designation mechanisms 318 may be located on the anterior side, the posterior side, and/or on the interior and/or exterior sidewalls of the prosthetic capsular device. In some embodiments, the one or more orientation designation mechanisms 318 may assist and/or allow a surgeon or medical professional to determine or perceive if the prosthetic capsular device is oriented correctly before, during, and/or after insertion into the eye. The orientation designation indicators or mechanisms 318 may comprise similar or identical features as those discussed in relation to orientation designation indicators or mechanisms 118 of FIG. 1.

In some embodiments, the device 300 may comprise an inner thickness 310 comprising a distance between an inner surface of the sidewall at the anterior opening and an inner surface of the sidewall at the posterior opening. In some embodiments, the inner thickness 310 may be about 0.98 mm.

In some embodiments, the taper length 314 may comprise a distance between the exterior surface of the sidewall at its most anterior/posterior point and the exterior surface at the anterior/posterior opening. In some embodiments, the exterior surface may comprise curved surfaces 322 and 316 at the ridge and at the openings, respectively. The curved shape of the sidewalls may contribute to a reduction in post-surgical complications through minimization of contact or the severity of contact between the device 300 and the iris. The curved shape of the sidewall of device 300 near the openings is shown in Detail B.

In some embodiments, the device 300 may comprise an inner diameter 306 comprising the distance between the interior surfaces of the sidewalls at the ridge. In some embodiments, the inner diameter 306 may be about 9.15 mm. In some embodiments, the interior diameter may be between about 5.00 mm and 15.00 mm.

In some embodiments, the device 300 may comprise a ridge thickness 312 comprising the size of the ridge locating ribs 305. In some embodiments, the ridge thickness 312 may be about 0.40 mm. In some embodiments, the ridge thickness 312 may be between about 0.10 mm and about 1.00 mm. In some embodiments, the ridge thickness may be configured to reduce the possibility of lens tilt by an intraocular lens located in the ridge and secured by ribs 305.

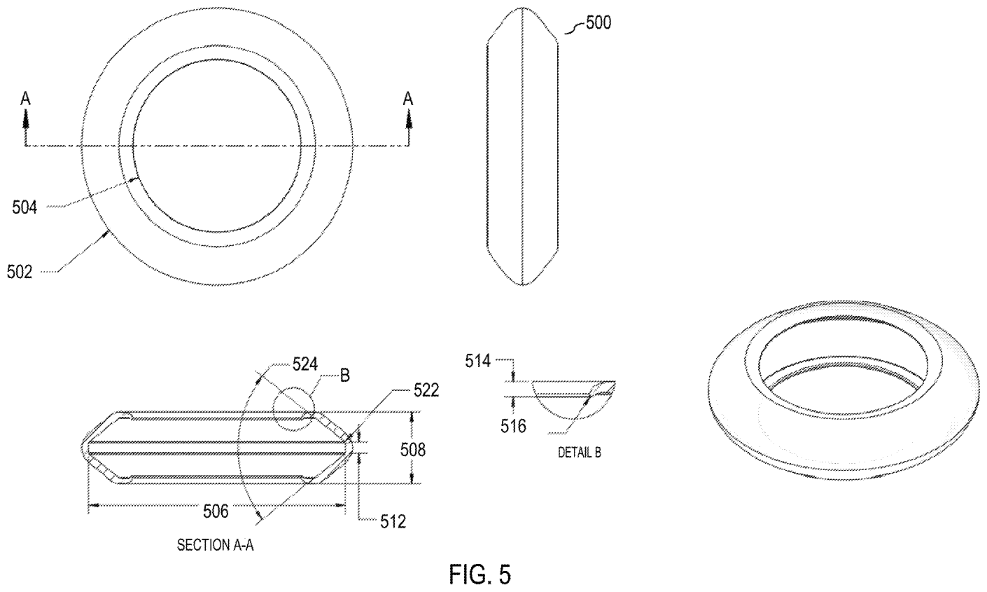

FIG. 5 illustrates another example prosthetic capsular device according to some embodiments herein. In device 500 of FIG. 5, the sidewall at the anterior portion of the device 500 and the sidewall at the posterior of the device 500 may form a sidewall angle 524, formed at a slot of the device 500. In some embodiments, the sidewall angle 524 may be larger than that of device 100 and device 300. For example, the sidewall angle may be about 750 or even larger. Furthermore, device 500 may not comprise any ribs, such as ribs 105 or ribs 305. Instead, device 500 may comprise a slot within the device cavity configured to secure an intraocular lens therein.

In some embodiments, a length of a major axis of the device 500 or a length measured from the outermost end of one sidewall to the outermost end of another sidewall along a major axis of the device 500 can be about 9.65 mm. In other embodiments, the length of the major axis of the device 500 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 500 may comprise a diameter 502 of the device 500.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 504, which can be a diameter of an anterior opening and/or a posterior opening.

In some embodiments, taper length 514 may comprise a distance between the exterior surface of the sidewall at its most anterior/posterior point and the exterior surface at the anterior/posterior opening. In some embodiments, the exterior surface may comprise curved surfaces 522 and 516 at the slot and at the openings, respectively. The curved shape of the sidewalls may contribute to a reduction in post-surgical complications through minimization of contact or the severity of contact between the device 500 and the iris. The curved shape of the sidewall of the device 500 near the openings is shown in Detail B.

In some embodiments, the device 500 may comprise an inner diameter 506 comprising the distance between the interior surfaces of the sidewalls at the ridge. In some embodiments, the inner diameter 506 may be about 9.15 mm. In some embodiments, the interior diameter may be between about 5.00 mm and 15.00 mm.

In some embodiments, the device 500 may comprise a slot thickness 512 comprising the size of the slot. In some embodiments, the slot thickness 512 may be about 0.40 mm. In some embodiments, the slot thickness 512 may be between about 0.10 mm and about 1.00 mm. In some embodiments, the slot thickness may be configured to reduce the possibility of lens tilt by an intraocular lens located in the slot.

In some embodiments, a thickness 508 of the device 500 may comprise a maximum distance between the anterior side and posterior side of the device 500. In some embodiments, the thickness 508 of the device 308 may be about 2.50 mm. In some embodiments, the thickness 508 of the device 500 may be between about 0.5 mm and 4.0 mm. In some embodiments, the thickness 508 of the device may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

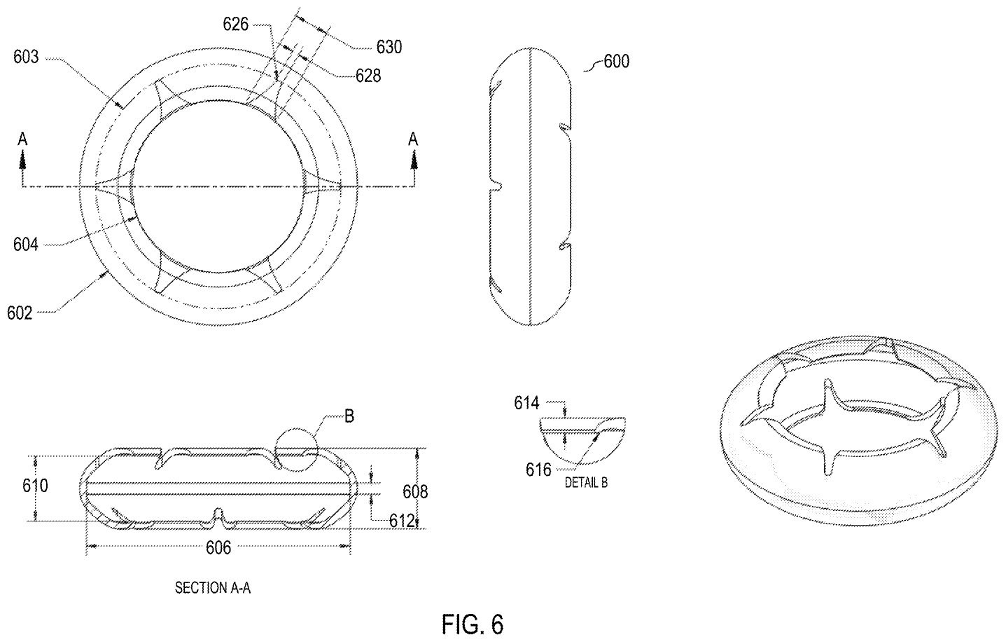

FIG. 6 illustrates another example prosthetic capsular device according to some embodiments herein. In device 600 of FIG. 6, the sidewall at the anterior portion of the device 600 and the sidewall at the posterior of the device 600 may comprise one or more cutouts 626, opening the anterior portion and the posterior portion of the device 600 to the interior cavity. In some embodiments, there may be 6 cutouts in the device 600. However, the number and shape of the cutouts is not limited. In some embodiments, the cutouts 626 may facilitate folding and expansion of the device or may allow for insertion of differently shaped or sized intraocular lenses.

In some embodiments, the cutouts may be substantially triangular with a rounded or blunted tip, wherein the tip comprises a width 628. In some embodiments, the tip may comprise a width 628 of about 0.25 mm. In some embodiments, the tip may comprise a width 628 of about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, and/or within a range defined by two of the aforementioned values. In some embodiments, the base of the cutouts may comprise a base width 630 of about 1.25 mm. In some embodiments, the base width 630 may range from about 0.1 mm to about 3.00 mm. In some embodiments, the tip may comprise one or more rounded corners having a radius of about 0.13 mm. In some embodiments, the diameter 603 of the cutouts 626, measured at the tips of the cutouts, may be about 8.5 mm.

In some embodiments, a length of a major axis of the device 600 or a length measured from the outermost end of one sidewall to the outermost end of another sidewall along a major axis of the device 600 can be about 9.65 mm. In other embodiments, the length of the major axis of the device 600 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 600 may comprise a diameter 602 of the device 600.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 604, which can be a diameter of an anterior opening and/or a posterior opening.

In some embodiments, device 600 may comprise an inner thickness 610 comprising a distance between an inner surface of the sidewall at the anterior opening and an inner surface of the sidewall at the posterior opening. In some embodiments, the inner thickness 610 may be about 2.27 mm.

In some embodiments, the taper length 614 may comprise a distance between the exterior surface of the sidewall at its most anterior/posterior point and the exterior surface at the anterior/posterior opening. In some embodiments, the exterior surface may comprise curved surface 616 at the openings. The curved shape of the sidewalls may contribute to a reduction in post-surgical complications through minimization of contact or the severity of contact between the device 600 and the iris. The curved shape of the sidewall of device 600 near the openings is shown in Detail B.

In some embodiments, device 600 may comprise an inner diameter 606 comprising the distance between the interior surfaces of the sidewalls at the ridge. In some embodiments, the inner diameter 606 may be about 9.15 mm. In some embodiments, the interior diameter may be between about 5.00 mm and 15.00 mm.

In some embodiments, the device 600 may comprise a slot thickness 612 comprising the size of the slot. In some embodiments, the slot thickness 612 may be about 0.40 mm. In some embodiments, the slot thickness 612 may be between about 0.10 mm and about 1.00 mm. In some embodiments, the slot thickness may be configured to reduce the possibility of lens tilt by an intraocular lens located in the slot.

In some embodiments, a thickness 608 of the device 600 may comprise a maximum distance between the anterior side and posterior side of the device 600. In some embodiments, the thickness 608 of the device 600 may be about 2.80 mm. In some embodiments, the thickness 608 of the device 600 may be between about 0.5 mm and 4.0 mm. In some embodiments, the thickness 608 of the device 600 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

In some embodiments, device 600 may comprise an inner thickness 610 comprising a distance between an inner surface of the sidewall at the anterior opening and an inner surface of the sidewall at the posterior opening. In some embodiments, the inner thickness 610 may be about 2.27 mm.

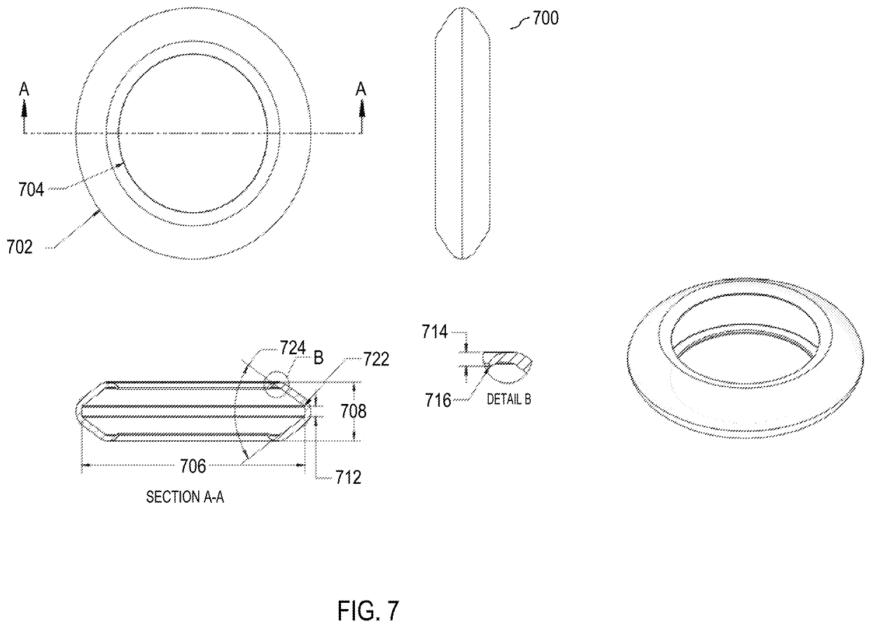

FIG. 7 illustrates another example prosthetic capsular device according to some embodiments herein. In device 700 of FIG. 7, the sidewall at the anterior portion of the device 700 and the sidewall at the posterior of the device 700 may form a sidewall angle 724, formed at a slot of the device 700. In some embodiments, the sidewall angle 724 may be larger than that of device 100 and device 300. For example, the sidewall angle may be about 690 or even larger. Furthermore, device 700 may not comprise any ribs, such as ribs 105 or ribs 305. Instead, device 700 may comprise a slot within the device cavity configured to secure an intraocular lens therein.

In some embodiments, a length of a major axis of the device 700 or a length measured from the outermost end of one sidewall to the outermost end of another sidewall along a major axis of the device 700 can be about 9.65 mm. In other embodiments, the length of the major axis of the device 700 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 700 may comprise a diameter 702 of the device 700.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 704, which can be a diameter of an anterior opening and/or a posterior opening.

In some embodiments, the taper length 714 may comprise a distance between the exterior surface of the sidewall at its most anterior/posterior point and the exterior surface at the anterior/posterior opening. In some embodiments, the exterior surface may comprise curved surfaces 722 and 716 at the slot and at the openings, respectively. The curved shape of the sidewalls may contribute to a reduction in post-surgical complications through minimization of contact or the severity of contact between the device 700 and the iris. The curved shape of the sidewall of device 700 near the openings is shown in Detail B.

In some embodiments, the device 700 may comprise an inner diameter 706 comprising the distance between the interior surfaces of the sidewalls at the ridge. In some embodiments, the inner diameter 706 may be about 9.15 mm. In some embodiments, the interior diameter may be between about 5.00 mm and 15.00 mm.

In some embodiments, the device 700 may comprise a slot thickness 712 comprising the size of the slot. In some embodiments, the slot thickness 712 may be about 0.40 mm. In some embodiments, the slot thickness 712 may be between about 0.10 mm and about 1.00 mm. In some embodiments, the slot thickness may be configured to reduce the possibility of lens tilt by an intraocular lens located in the slot.

In some embodiments, a thickness 708 of the device 700 may comprise a maximum distance between the anterior side and posterior side of the device 700. In some embodiments, the thickness 708 of the device 700 may be about 2.21 mm. In some embodiments, the thickness 708 of the device 700 may be between about 0.5 mm and 4.0 mm. In some embodiments, the thickness 708 of the device 700 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

In some embodiments, the device 700 may comprise an inner thickness 710 comprising a distance between an inner surface of the sidewall at the anterior opening and an inner surface of the sidewall at the posterior opening. In some embodiments, the inner thickness 710 may be about 2.27 mm.

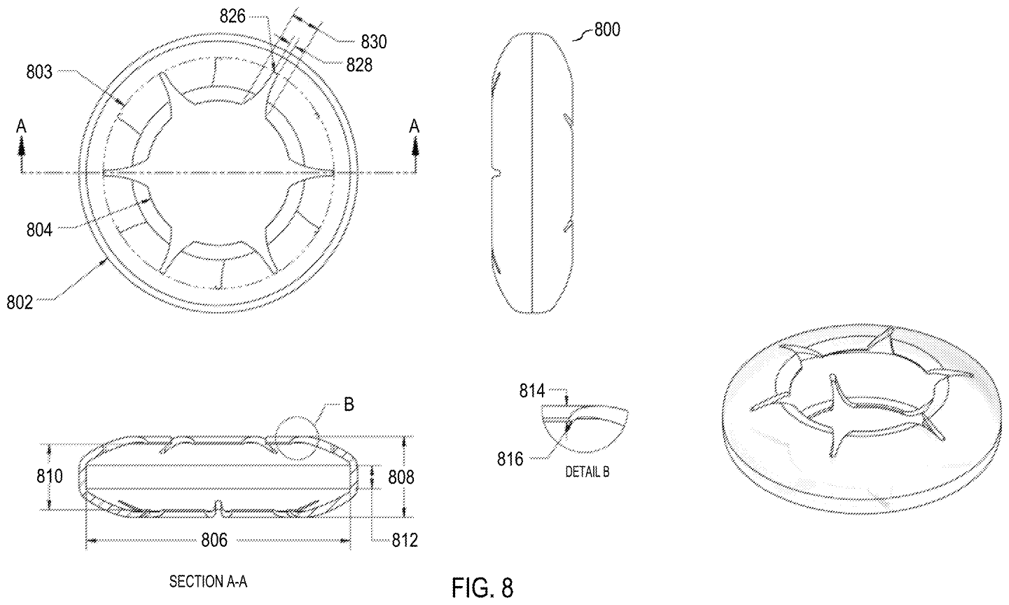

FIG. 8 illustrates another example prosthetic capsular device according to some embodiments herein. In device 800 of FIG. 8, the sidewall at the anterior portion of the device 800 and the sidewall at the posterior of the device 800 may comprise one or more cutouts 826, opening the anterior portion and the posterior portion of the device 800 to the interior cavity. In some embodiments, there may be 6 cutouts in the device 800. However, the number and shape of the cutouts is not limited. In some embodiments, the cutouts 826 may facilitate folding and expansion of the device or may allow for insertion of differently shaped or sized intraocular lenses.

In some embodiments, the cutouts may be substantially triangular with a rounded or blunted tip, wherein the tip comprises a width 828. In some embodiments, the tip may comprise a width 828 of about 0.19 mm. In some embodiments, the tip may comprise a width 828 of about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, and/or within a range defined by two of the aforementioned values. In some embodiments, the base of the cutouts may comprise a base width 830 of about 0.98 mm. In some embodiments, the base width 830 may range from about 0.1 mm to about 3.00 mm. In some embodiments, the tip may comprise one or more rounded corners having a radius of about 0.10 mm. In some embodiments, the diameter 803 of the cutouts 826, measured at the tips of the cutouts, may be about 8.00 mm.

In some embodiments, a length of a major axis of the device 800 or a length measured from the outermost end of one sidewall to the outermost end of another sidewall along a major axis of the device 800 can be about 9.65 mm. In other embodiments, the length of the major axis of the device 800 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 800 may comprise a diameter 802 of the device 800.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 804, which can be a diameter of an anterior opening and/or a posterior opening.

In some embodiments, the device 800 may comprise an inner thickness 810 comprising a distance between an inner surface of the sidewall at the anterior opening and an inner surface of the sidewall at the posterior opening. In some embodiments, the inner thickness 810 may be about 2.27 mm.

In some embodiments, the taper length 814 may comprise a distance between the exterior surface of the sidewall at its most anterior/posterior point and the exterior surface at the anterior/posterior opening. In some embodiments, the exterior surface may comprise curved surfaces 816 at the openings. The curved shape of the sidewalls may contribute to a reduction in post-surgical complications through minimization of contact or the severity of contact between the device 800 and the iris. The curved shape of the sidewall of device 800 near the openings is shown in Detail B.

In some embodiments, the device 800 may comprise an inner diameter 806 comprising the distance between the interior surfaces of the sidewalls at the ridge. In some embodiments, the inner diameter 806 may be about 9.15 mm. In some embodiments, the interior diameter may be between about 5.00 mm and 15.00 mm.

In some embodiments, the device 800 may comprise a slot thickness 812 comprising the size of the slot. In some embodiments, the slot thickness 812 may be about 0.80 mm. In some embodiments, the slot thickness 812 may be between about 0.10 mm and about 1.00 mm. In some embodiments, the slot thickness may be configured to reduce the possibility of lens tilt by an intraocular lens located in the slot.

In some embodiments, a thickness 808 of the device 800 may comprise a maximum distance between the anterior side and posterior side of the device 800. In some embodiments, the thickness 808 of the device 800 may be about 2.80 mm. In some embodiments, the thickness 808 of the device 800 may be between about 0.5 mm and 4.0 mm. In some embodiments, the thickness 808 of the device 800 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

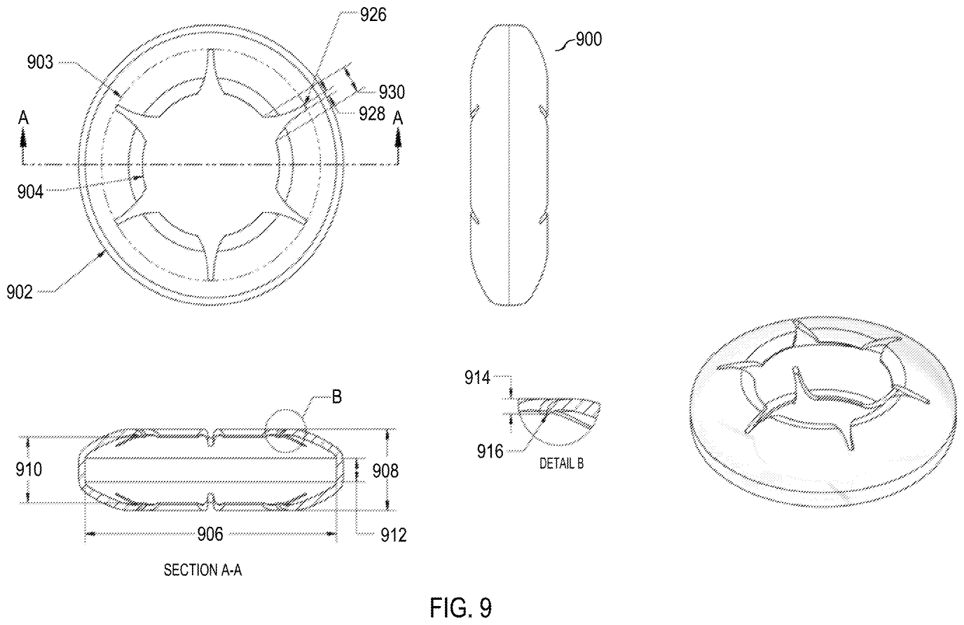

FIG. 9 illustrates another example prosthetic capsular device according to some embodiments herein. In device 900 of FIG. 9, the sidewall at the anterior portion of the device 900 and the sidewall at the posterior of the device 900 may comprise one or more cutouts 926, opening the anterior portion and the posterior portion of the device 900 to the interior cavity. In some embodiments, there may be 6 cutouts in the device 900. However, the number and shape of the cutouts is not limited. In some embodiments, the cutouts 926 may facilitate folding and expansion of the device or may allow for insertion of differently shaped or sized intraocular lenses.

In some embodiments, the cutouts may be substantially triangular with a rounded or blunted tip, wherein the tip comprises a width 928. In some embodiments, the tip may comprise a width 928 of about 0.19 mm. In some embodiments, the tip may comprise a width 928 of about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, and/or within a range defined by two of the aforementioned values. In some embodiments, the base of the cutouts may comprise a base width 930 of about 0.98 mm. In some embodiments, the base width 930 may range from about 0.1 mm to about 3.00 mm. In some embodiments, the tip may comprise one or more rounded corners having a radius of about 0.10 mm. In some embodiments, the diameter 903 of the cutouts 926, measured at the tips of the cutouts, may be about 8.00 mm.

In some embodiments, a length of a major axis of the device 900 or a length measured from the outermost end of one sidewall to the outermost end of another sidewall along a major axis of the device 900 can be about 9.65 mm. In other embodiments, the length of the major axis of the device 900 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 900 may comprise a diameter 902 of the device 900.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 904, which can be a diameter of an anterior opening and/or a posterior opening.

In some embodiments, the device 900 may comprise an inner thickness 910 comprising a distance between an inner surface of the sidewall at the anterior opening and an inner surface of the sidewall at the posterior opening. In some embodiments, the inner thickness 910 may be about 2.27 mm.

In some embodiments, the taper length 914 may comprise a distance between the exterior surface of the sidewall at its most anterior/posterior point and the exterior surface at the anterior/posterior opening. In some embodiments, the exterior surface may comprise curved surfaces 916 at the openings. The curved shape of the sidewalls may contribute to a reduction in post-surgical complications through minimization of contact or the severity of contact between the device 900 and the iris. The curved shape of the sidewall of device 900 near the openings is shown in Detail B.

In some embodiments, the device 900 may comprise an inner diameter 906 comprising the distance between the interior surfaces of the sidewalls at the ridge. In some embodiments, the inner diameter 906 may be about 9.15 mm. In some embodiments, the interior diameter may be between about 5.00 mm and 15.00 mm.

In some embodiments, the device 900 may comprise a slot thickness 912 comprising the size of the slot. In some embodiments, the slot thickness 912 may be about 0.80 mm. In some embodiments, the slot thickness 912 may be between about 0.10 mm and about 1.00 mm. In some embodiments, the slot thickness may be configured to reduce the possibility of lens tilt by an intraocular lens located in the slot.

In some embodiments, a thickness 908 of the device 900 may comprise a maximum distance between the anterior side and posterior side of the device 900. In some embodiments, the thickness 908 of the device 900 may be about 2.80 mm. In some embodiments, the thickness 908 of the device 900 may be between about 0.5 mm and 4.0 mm. In some embodiments, the thickness 908 of the device 900 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

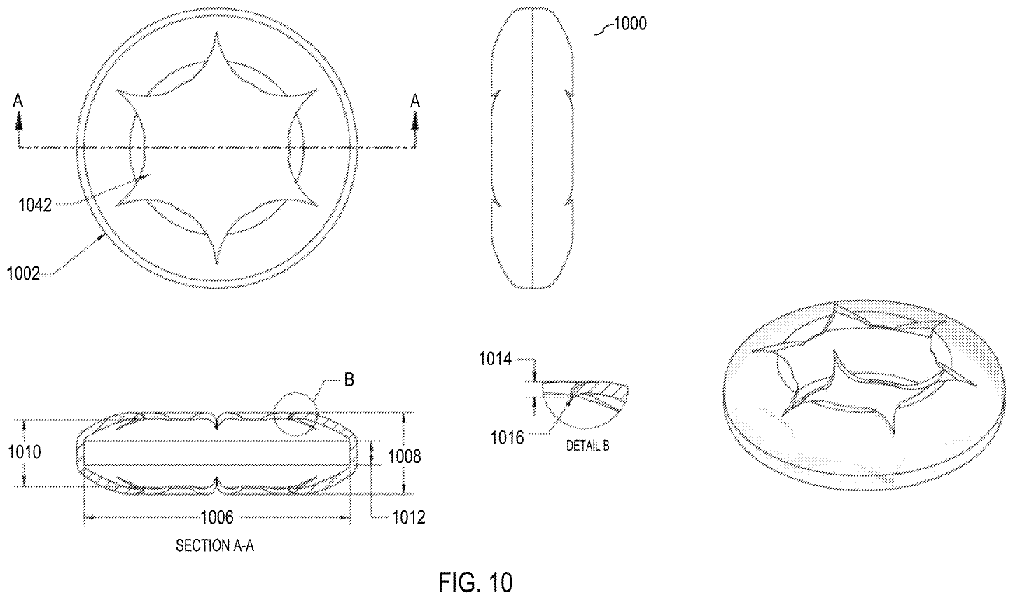

FIG. 10 illustrates another example prosthetic capsular device according to some embodiments herein. In device 1000 of FIG. 10, the sidewall at the anterior portion of the device 1000 and the sidewall at the posterior of the device 1000 may comprise one or more cutouts, opening the anterior portion and the posterior portion of the device 1000 to the interior cavity. In some embodiments, there may be 6 cutouts in the device 1000. However, the number and shape of the cutouts is not limited. In some embodiments, the cutouts may facilitate folding and expansion of the device or may allow for insertion of differently shaped or sized intraocular lenses. In some embodiments, the cutouts may be integrally formed with the anterior opening and the posterior opening forming a pointed star-shaped opening.

In some embodiments, a length of a major axis of the device 1000 or a length measured from the outermost end of one sidewall to the outermost end of another sidewall along a major axis of the device 1000 can be about 9.65 mm. In other embodiments, the length of the major axis of the device 1000 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 1000 may comprise a diameter 1002 of the device 1000.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 1004, which can be a diameter of an anterior opening and/or a posterior opening.

In some embodiments, the device 1000 may comprise an inner thickness 1010 comprising a distance between an inner surface of the sidewall at the anterior opening and an inner surface of the sidewall at the posterior opening. In some embodiments, the inner thickness 1010 may be about 2.27 mm.

In some embodiments, the taper length 1014 may comprise a distance between the exterior surface of the sidewall at its most anterior/posterior point and the exterior surface at the anterior/posterior opening. In some embodiments, the exterior surface may comprise curved surface 1016 at the openings, respectively. The curved shape of the sidewalls may contribute to a reduction in post-surgical complications through minimization of contact or the severity of contact between the device 1000 and the iris. The curved shape of the sidewall of the device 1000 near the openings is shown in Detail B.

In some embodiments, device 1000 may comprise an inner diameter 1006 comprising the distance between the interior surfaces of the sidewalls at the ridge. In some embodiments, the inner diameter 1006 may be about 9.15 mm. In some embodiments, the interior diameter may be between about 5.00 mm and 15.00 mm.

In some embodiments, the device 1000 may comprise a slot thickness 1012 comprising the size of the slot. In some embodiments, the slot thickness 1012 may be about 0.80 mm. In some embodiments, the slot thickness 1012 may be between about 0.10 mm and about 1.00 mm. In some embodiments, the slot thickness may be configured to reduce the possibility of lens tilt by an intraocular lens located in the slot.

In some embodiments, a thickness 1008 of the device 1000 may comprise a maximum distance between the anterior side and posterior side of the device 1000. In some embodiments, the thickness 1008 of the device 1000 may be about 2.80 mm. In some embodiments, the thickness 1008 of the device 1000 may be between about 0.5 mm and 4.0 mm. In some embodiments, the thickness 1008 of the device 1000 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

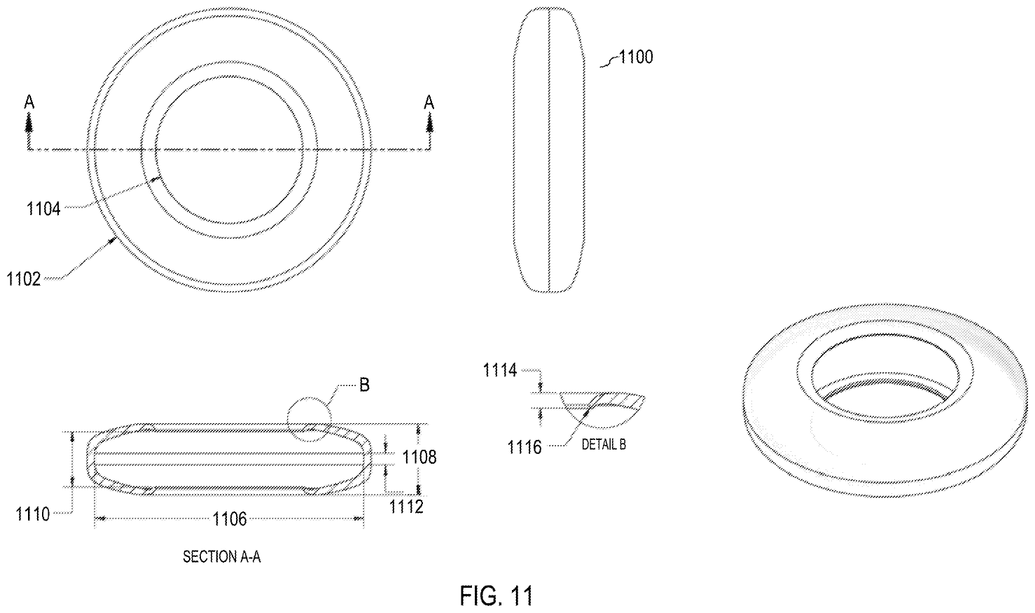

FIG. 11 illustrates another example prosthetic capsular device according to some embodiments herein. In device 1100 of FIG. 11, the sidewall at the anterior portion of the device 1100 and the sidewall at the posterior of the device 1100 may form a slot of the device 1100. In some embodiments, the device 1100 may comprise a cylindrical middle portion perpendicular to the anterior opening and the posterior opening. Device 1100 may comprise a slot within the device cavity configured to secure an intraocular lens therein.

In some embodiments, a length of a major axis of the device 1100 or a length measured from the outermost end of one sidewall to the outermost end of another sidewall along a major axis of the device 1100 can be about 9.65 mm. In other embodiments, the length of the major axis of the device 1100 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 1100 may comprise a diameter 1102 of the device 1100.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 1104, which can be a diameter of an anterior opening and/or a posterior opening.

In some embodiments, the taper length 1114 may comprise a distance between the exterior surface of the sidewall at its most anterior/posterior point and the exterior surface at the anterior/posterior opening. In some embodiments, the exterior surface may comprise curved surfaces 1116 at the openings. The curved shape of the sidewalls may contribute to a reduction in post-surgical complications through minimization of contact or the severity of contact between the device 1100 and the iris. The curved shape of the sidewall of the device 1100 near the openings is shown in Detail B.

In some embodiments, the device 1100 may comprise an inner diameter 1106 comprising the distance between the interior surfaces of the sidewalls at the ridge. In some embodiments, the inner diameter 1106 may be about 9.15 mm. In some embodiments, the interior diameter may be between about 5.00 mm and 15.00 mm.

In some embodiments, device 1100 may comprise a slot thickness 1112 comprising the size of the slot. In some embodiments, the slot thickness 1112 may be about 0.40 mm. In some embodiments, the slot thickness 1112 may be between about 0.10 mm and about 1.00 mm. In some embodiments, the slot thickness may be configured to reduce the possibility of lens tilt by an intraocular lens located in the slot.

In some embodiments, a thickness 1108 of the device 1100 may comprise a maximum distance between the anterior side and posterior side of the device 1100. In some embodiments, the thickness 1108 of the device 1100 may be about 2.40 mm. In some embodiments, the thickness 1108 of the device 1100 may be between about 0.5 mm and 4.0 mm. In some embodiments, the thickness 1108 of the device 1100 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

In some embodiments, the device 1100 may comprise an inner thickness 1110 comprising a distance between an inner surface of the sidewall at the anterior opening and an inner surface of the sidewall at the posterior opening. In some embodiments, the inner thickness 1110 may be about 1.87 mm.

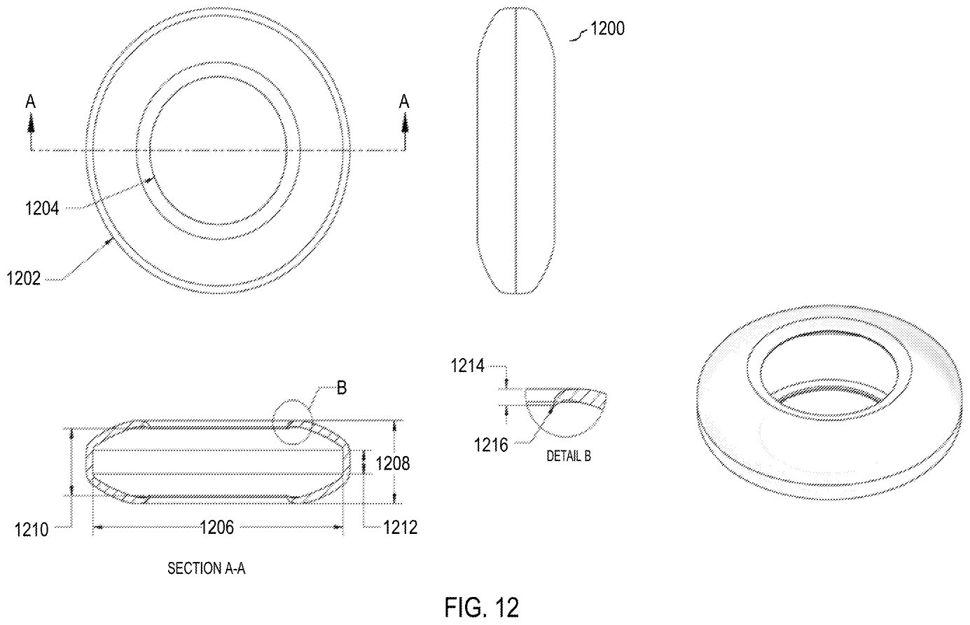

FIG. 12 illustrates another example prosthetic capsular device according to some embodiments herein. In device 1200 of FIG. 12, the sidewall at the anterior portion of device 1200 and the sidewall at the posterior of the device 1200 may form a slot of the device 1200. In some embodiments, the device 1200 may comprise a cylindrical middle portion perpendicular to the anterior opening and the posterior opening. Device 1200 may comprise a slot within the device cavity configured to secure an intraocular lens therein.

In some embodiments, a length of a major axis of the device 1200 or a length measured from the outermost end of one sidewall to the outermost end of another sidewall along a major axis of the device 1200 can be about 9.65 mm. In other embodiments, the length of the major axis of the device 1200 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 1200 may comprise a diameter 1202 of the device 1200.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 1204, which can be a diameter of an anterior opening and/or a posterior opening.

In some embodiments, the taper length 1214 may comprise a distance between the exterior surface of the sidewall at its most anterior/posterior point and the exterior surface at the anterior/posterior opening. In some embodiments, the exterior surface may comprise curved surfaces 1216 at the openings. The curved shape of the sidewalls may contribute to a reduction in post-surgical complications through minimization of contact or the severity of contact between the device 1200 and the iris. The curved shape of the sidewall of device 1200 near the openings is shown in Detail B.

In some embodiments, device 1200 may comprise an inner diameter 1206 comprising the distance between the interior surfaces of the sidewalls at the ridge. In some embodiments, the inner diameter 1206 may be about 9.15 mm. In some embodiments, the interior diameter may be between about 5.00 mm and 15.00 mm.

In some embodiments, device 1200 may comprise a slot thickness 1212 comprising the size of the slot. In some embodiments, the slot thickness 1212 may be about 0.80 mm. In some embodiments, the slot thickness 1212 may be between about 0.10 mm and about 1.00 mm. In some embodiments, the slot thickness may be configured to reduce the possibility of lens tilt by an intraocular lens located in the slot.

In some embodiments, a thickness 1208 of the device 1200 may comprise a maximum distance between the anterior side and posterior side of the device 1200. In some embodiments, the thickness 1208 of the device 1200 may be about 2.80 mm. In some embodiments, the thickness 1208 of the device 1200 may be between about 0.5 mm and 4.0 mm. In some embodiments, the thickness 1208 of the device 1200 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

In some embodiments, the device 1200 may comprise an inner thickness 1210 comprising a distance between an inner surface of the sidewall at the anterior opening and an inner surface of the sidewall at the posterior opening. In some embodiments, the inner thickness 1210 may be about 2.27 mm.

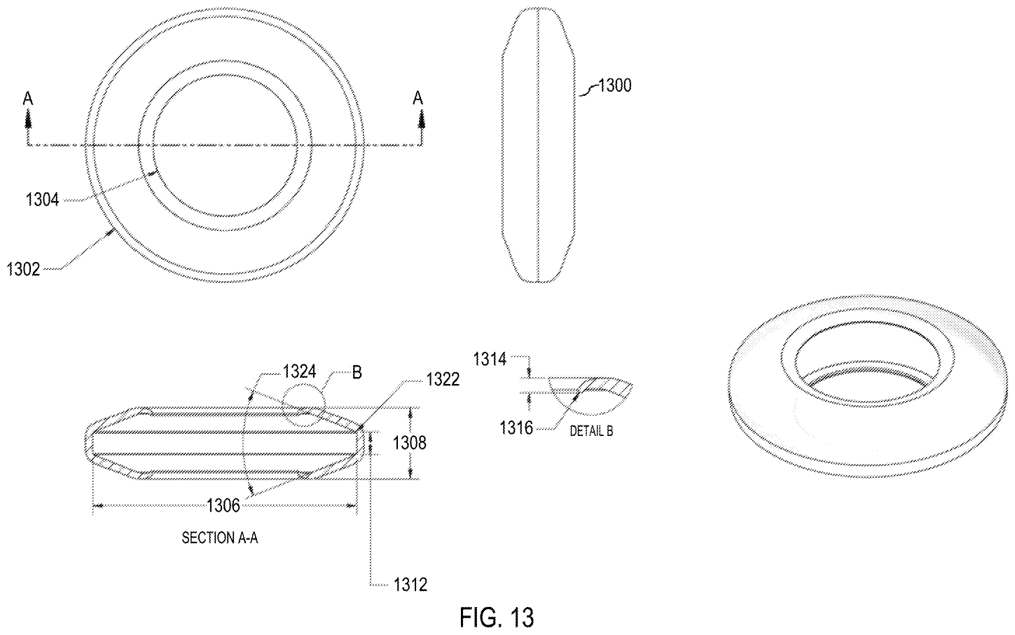

FIG. 13 illustrates another example prosthetic capsular device according to some embodiments herein. In device 1300 of FIG. 13, the sidewall at the anterior portion of the device 1300 and the sidewall at the posterior of the device 1300 may form a sidewall angle 1324, formed at a slot of the device 1300. For example, the sidewall angle may be about 44°. Furthermore, device 1300 may not comprise any ribs, such as ribs 105 or ribs 305. Instead, device 1300 may comprise a slot within the device cavity configured to secure an intraocular lens therein.

In some embodiments, a length of a major axis of the device 1300 or a length measured from the outermost end of one sidewall to the outermost end of another sidewall along a major axis of the device 1300 can be about 9.65 mm. In other embodiments, the length of the major axis of the device 1300 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 1300 may comprise a diameter 1302 of the device 1300.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 1304, which can be a diameter of an anterior opening and/or a posterior opening.

In some embodiments, the taper length 1314 may comprise a distance between the exterior surface of the sidewall at its most anterior/posterior point and the exterior surface at the anterior/posterior opening. In some embodiments, the exterior surface may comprise curved surfaces 1322 and 1316 at the slot and at the openings, respectively. The curved shape of the sidewalls may contribute to a reduction in post-surgical complications through minimization of contact or the severity of contact between device 1300 and the iris. The curved shape of the sidewall of device 1300 near the openings is shown in Detail B.

In some embodiments, the device 1300 may comprise an inner diameter 1306 comprising the distance between the interior surfaces of the sidewalls at the ridge. In some embodiments, the inner diameter 1306 may be about 9.15 mm. In some embodiments, the interior diameter may be between about 5.00 mm and 15.00 mm.

In some embodiments, the device 1300 may comprise a slot thickness 1312 comprising the size of the slot. In some embodiments, the slot thickness 1312 may be about 0.80 mm. In some embodiments, the slot thickness 1312 may be between about 0.10 mm and about 1.00 mm. In some embodiments, the slot thickness may be configured to reduce the possibility of lens tilt by an intraocular lens located in the slot.

In some embodiments, a thickness 1308 of the device 1300 may comprise a maximum distance between the anterior side and posterior side of the device 1300. In some embodiments, the thickness 1308 of the device 1300 may be about 2.50 mm. In some embodiments, the thickness 1308 of device 1300 may be between about 0.5 mm and 4.0 mm. In some embodiments, the thickness 1308 of the device 1300 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

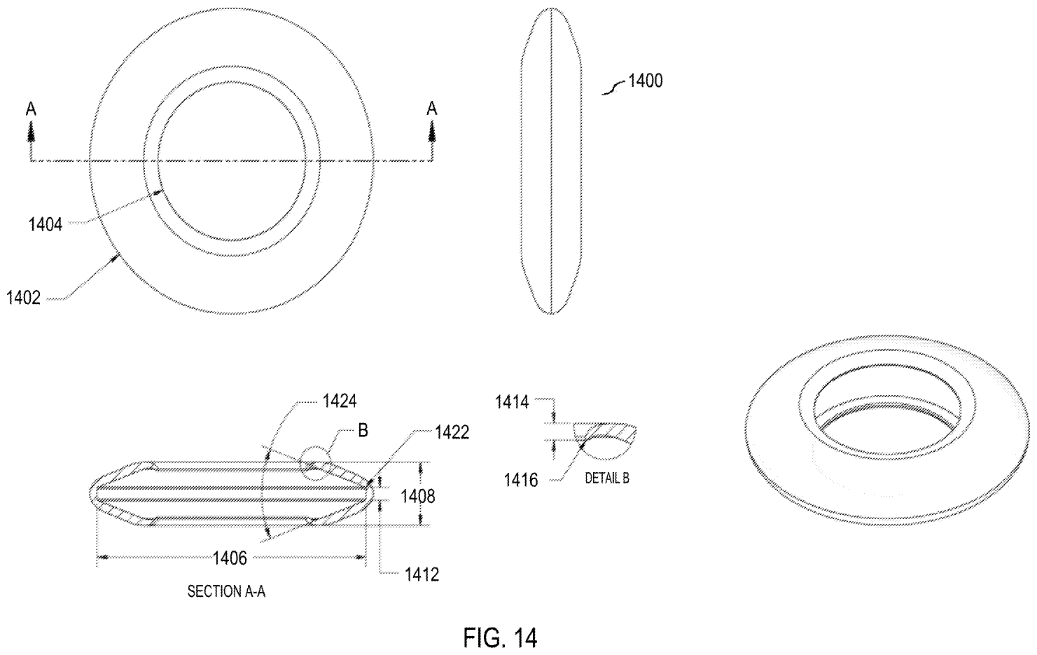

FIG. 14 illustrates another example prosthetic capsular device according to some embodiments herein. In device 1400 of FIG. 14, the sidewall at the anterior portion of the device 1400 and the sidewall at the posterior of the device 1400 may form a sidewall angle 1424, formed at a slot of the device 1400. For example, the sidewall angle may be about 41°. Furthermore, device 1400 may not comprise any ribs, such as ribs 105 or ribs 305. Instead, device 1400 may comprise a slot within the device cavity configured to secure an intraocular lens therein.

In some embodiments, a length of a major axis of the device 1400 or a length measured from the outermost end of one sidewall to the outermost end of another sidewall along a major axis of the device 1400 can be about 9.65 mm. In other embodiments, the length of the major axis of the device 1400 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 1400 may comprise a diameter 1402 of the device 1400.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 1404, which can be a diameter of an anterior opening and/or a posterior opening.

In some embodiments, the taper length 1414 may comprise a distance between the exterior surface of the sidewall at its most anterior/posterior point and the exterior surface at the anterior/posterior opening. In some embodiments, the exterior surface may comprise curved surfaces 1422 and 1416 at the slot and at the openings, respectively. The curved shape of the sidewalls may contribute to a reduction in post-surgical complications through minimization of contact or the severity of contact between the device 1400 and the iris. The curved shape of the sidewall of device 1400 near the openings is shown in Detail B.

In some embodiments, the device 1400 may comprise an inner diameter 1406 comprising the distance between the interior surfaces of the sidewalls at the ridge. In some embodiments, the inner diameter 1406 may be about 9.15 mm. In some embodiments, the interior diameter may be between about 5.00 mm and 15.00 mm.

In some embodiments, the device 1400 may comprise a slot thickness 1412 comprising the size of the slot. In some embodiments, the slot thickness 1412 may be about 0.40 mm. In some embodiments, the slot thickness 1412 may be between about 0.10 mm and about 1.00 mm. In some embodiments, the slot thickness may be configured to reduce the possibility of lens tilt by an intraocular lens located in the slot.

In some embodiments, a thickness 1408 of the device 1400 may comprise a maximum distance between the anterior side and posterior side of the device 1400. In some embodiments, the thickness 1408 of the device 1400 may be about 2.00 mm. In some embodiments, the thickness 1408 of the device 1400 may be between about 0.5 mm and 4.0 mm. In some embodiments, the thickness 1408 of the device 1400 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

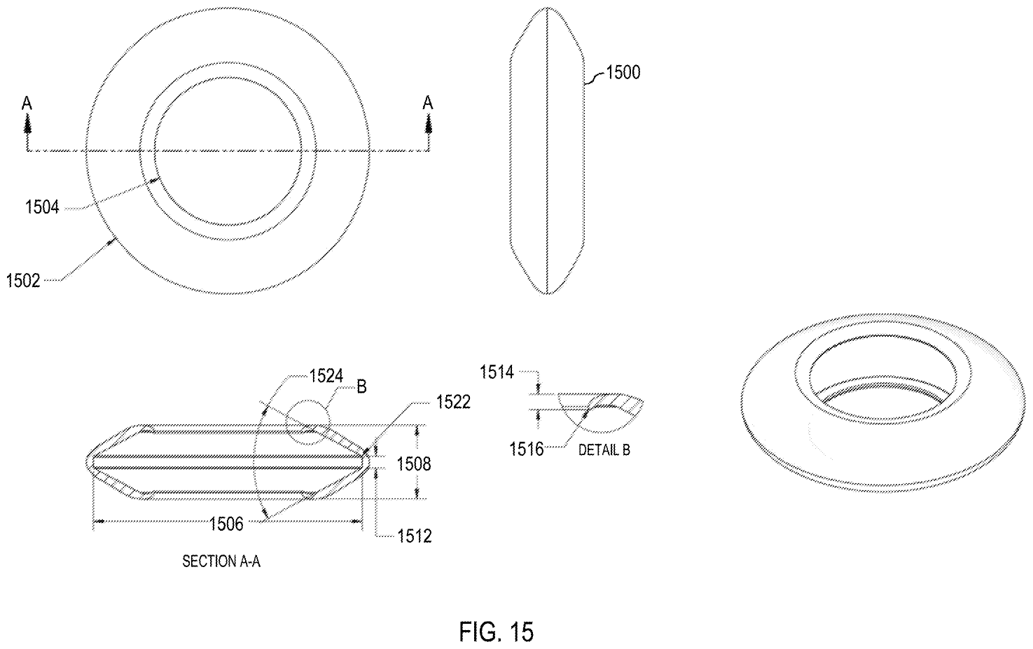

FIG. 15 illustrates another example prosthetic capsular device according to some embodiments herein. In device 1500 of FIG. 15, the sidewall at the anterior portion of the device 1500 and the sidewall at the posterior of the device 1500 may form a sidewall angle 1524, formed at a slot of the device 1500. For example, the sidewall angle may be about 56°. Furthermore, device 1500 may not comprise any ribs, such as ribs 105 or ribs 305. Instead, device 1500 may comprise a slot within the device cavity configured to secure an intraocular lens therein.

In some embodiments, a length of a major axis of the device 1500 or a length measured from the outermost end of one sidewall to the outermost end of another sidewall along a major axis of the device 1500 can be about 9.65 mm. In other embodiments, the length of the major axis of the device 1500 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of device 1500 may comprise a diameter 1502 of the device 1500.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 1504, which can be a diameter of an anterior opening and/or a posterior opening.

In some embodiments, the taper length 1514 may comprise a distance between the exterior surface of the sidewall at its most anterior/posterior point and the exterior surface at the anterior/posterior opening. In some embodiments, the exterior surface may comprise curved surfaces 1522 and 1516 at the slot and at the openings, respectively. The curved shape of the sidewalls may contribute to a reduction in post-surgical complications through minimization of contact or the severity of contact between device 1500 and the iris. The curved shape of the sidewall of device 1500 near the openings is shown in Detail B.

In some embodiments, the device 1500 may comprise an inner diameter 1506 comprising the distance between the interior surfaces of the sidewalls at the ridge. In some embodiments, the inner diameter 1506 may be about 9.15 mm. In some embodiments, the interior diameter may be between about 5.00 mm and 15.00 mm.

In some embodiments, device 1500 may comprise a slot thickness 1512 comprising the size of the slot. In some embodiments, the slot thickness 1512 may be about 0.40 mm. In some embodiments, the slot thickness 1512 may be between about 0.10 mm and about 1.00 mm. In some embodiments, the slot thickness may be configured to reduce the possibility of lens tilt by an intraocular lens located in the slot.

In some embodiments, a thickness 1508 of the device 1500 may comprise a maximum distance between the anterior side and posterior side of the device 1500. In some embodiments, the thickness 1508 of the device 1500 may be about 2.50 mm. In some embodiments, the thickness 1508 of device 1500 may be between about 0.5 mm and 4.0 mm. In some embodiments, the thickness 1508 of the device 1500 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

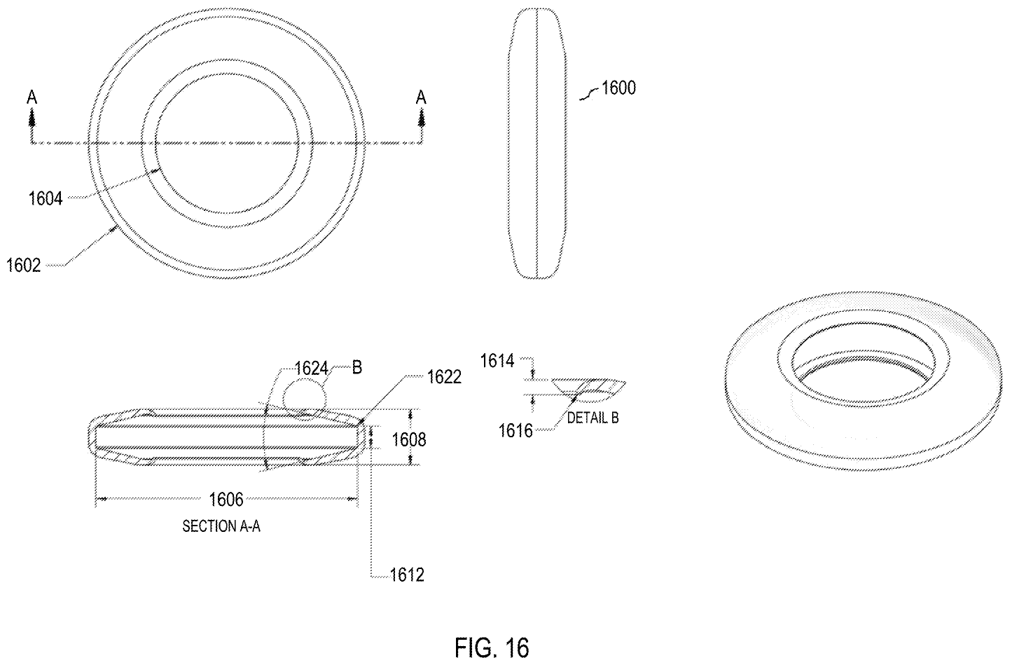

FIG. 16 illustrates another example prosthetic capsular device according to some embodiments herein. In device 1600 of FIG. 16, the sidewall at the anterior portion of device 1600 and the sidewall at the posterior of device 1600 may form a slot of device 1600. In some embodiments, device 1600 may comprise a cylindrical middle portion perpendicular to the anterior opening and the posterior opening. Device 1600 may comprise a slot within the device cavity configured to secure an intraocular lens therein. In device 1600 of FIG. 16, the sidewall at the anterior portion of the device 1600 and the sidewall at the posterior of the device 1600 may form a sidewall angle 1624, formed at a slot of the device 1600. For example, the sidewall angle may be about 27°. Furthermore, device 1600 may not comprise any ribs, such as ribs 105 or ribs 305. Instead, device 1600 may comprise a slot within the device cavity configured to secure an intraocular lens therein.

In some embodiments, a length of a major axis of the device 1600 or a length measured from the outermost end of one sidewall to the outermost end of another sidewall along a major axis of the device 1600 can be about 9.65 mm. In other embodiments, the length of the major axis of the device 1600 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 1600 may comprise a diameter 1602 of the device 1600.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 1604, which can be a diameter of an anterior opening and/or a posterior opening.

In some embodiments, the taper length 1614 may comprise a distance between the exterior surface of the sidewall at its most anterior/posterior point and the exterior surface at the anterior/posterior opening. In some embodiments, the exterior surface may comprise curved surfaces 1622 and 1616 at the slot and the openings, respectively. The curved shape of the sidewalls may contribute to a reduction in post-surgical complications through minimization of contact or the severity of contact between device 1600 and the iris. The curved shape of the sidewall of device 1600 near the openings is shown in Detail B.

In some embodiments, the device 1600 may comprise an inner diameter 1606 comprising the distance between the interior surfaces of the sidewalls at the ridge. In some embodiments, the inner diameter 1606 may be about 9.15 mm. In some embodiments, the interior diameter may be between about 5.00 mm and 15.00 mm.

In some embodiments, the device 1600 may comprise a slot thickness 1612 comprising the size of the slot. In some embodiments, the slot thickness 1612 may be about 0.80 mm. In some embodiments, the slot thickness 1612 may be between about 0.10 mm and about 1.00 mm. In some embodiments, the slot thickness may be configured to reduce the possibility of lens tilt by an intraocular lens located in the slot.

In some embodiments, a thickness 1608 of the device 1600 may comprise a maximum distance between the anterior side and posterior side of the device 1600. In some embodiments, the thickness 1608 of the device 1600 may be about 2.00 mm. In some embodiments, the thickness 1608 of the device 1600 may be between about 0.5 mm and 4.0 mm. In some embodiments, the thickness 1608 of the device 1600 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

FIG. 17 illustrates another example prosthetic capsular device according to some embodiments herein. In device 1700 of FIG. 17, the sidewall at the anterior portion of the device 1700 and the sidewall at the posterior of the device 1700 may form a slot of the device 1700. In some embodiments, the device 1700 may comprise a cylindrical middle portion perpendicular to the anterior opening and the posterior opening. Device 1700 may comprise a slot within the device cavity configured to secure an intraocular lens therein. In device 1700 of FIG. 17, the sidewall at the anterior portion of the device 1700 and the sidewall at the posterior of the device 1700 may form a sidewall angle 1724, formed at a slot of the device 1700. For example, the sidewall angle may be about 58°. Furthermore, device 1700 may not comprise any ribs, such as ribs 105 or ribs 305. Instead, device 1700 may comprise a slot within the device cavity configured to secure an intraocular lens therein.

In some embodiments, a length of a major axis of the device 1700 or a length measured from the outermost end of one sidewall to the outermost end of another sidewall along a major axis of the device 1700 can be about 9.65 mm. In other embodiments, the length of the major axis of the device 1700 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 1700 may comprise a diameter 1702 of the device 1700.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 1704, which can be a diameter of an anterior opening and/or a posterior opening.

In some embodiments, the taper length 1714 may comprise a distance between the exterior surface of the sidewall at its most anterior/posterior point and the exterior surface at the anterior/posterior opening. In some embodiments, the exterior surface may comprise curved surfaces 1722 and 1716 at the slot and the openings, respectively. The curved shape of the sidewalls may contribute to a reduction in post-surgical complications through minimization of contact or the severity of contact between the device 1700 and the iris. The curved shape of the sidewall of device 1700 near the openings is shown in Detail B.

In some embodiments, the device 1700 may comprise an inner diameter 1706 comprising the distance between the interior surfaces of the sidewalls at the ridge. In some embodiments, the inner diameter 1706 may be about 9.15 mm. In some embodiments, the interior diameter may be between about 5.00 mm and 15.00 mm.

In some embodiments, device 1700 may comprise a slot thickness 1712 comprising the size of the slot. In some embodiments, the slot thickness 1712 may be about 0.80 mm. In some embodiments, the slot thickness 1712 may be between about 0.10 mm and about 1.00 mm. In some embodiments, the slot thickness may be configured to reduce the possibility of lens tilt by an intraocular lens located in the slot.

In some embodiments, a thickness 1708 of the device 1700 may comprise a maximum distance between the anterior side and posterior side of the device 1700. In some embodiments, the thickness 1708 of the device 1700 may be about 3.00 mm. In some embodiments, the thickness 1708 of the device 1700 may be between about 0.5 mm and 4.0 mm. In some embodiments, the thickness 1708 of the device 1700 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

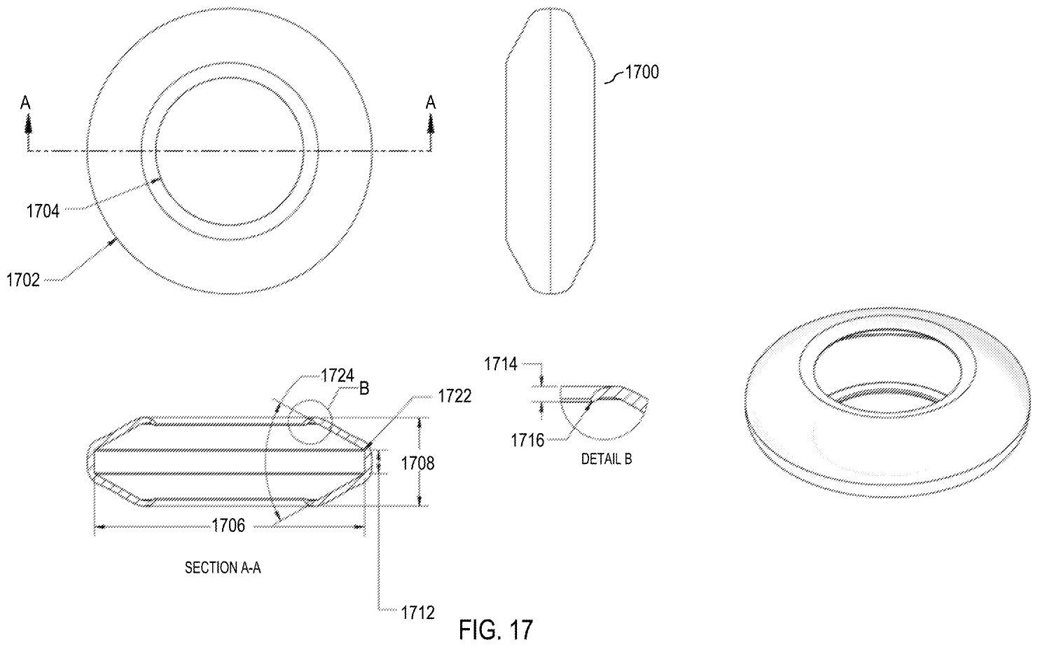

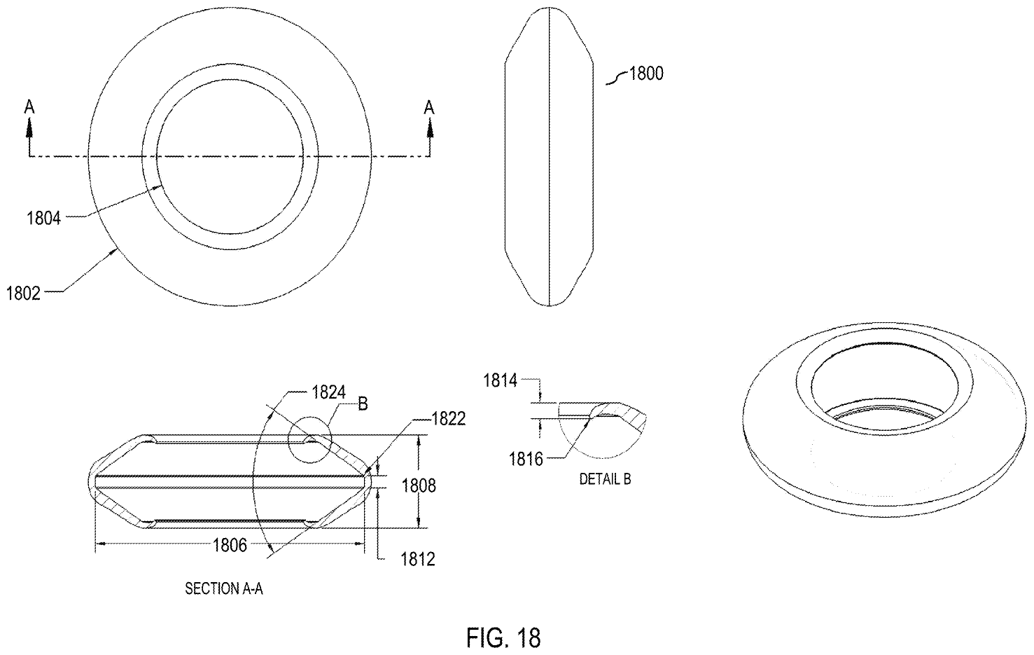

FIG. 18 illustrates another example prosthetic capsular device according to some embodiments herein. In device 1800 of FIG. 18, the sidewall at the anterior portion of the device 1800 and the sidewall at the posterior of the device 1800 may form a slot of the device 1800. In some embodiments, the device 1800 may comprise a cylindrical middle portion perpendicular to the anterior opening and the posterior opening. Device 1800 may comprise a slot within the device cavity configured to secure an intraocular lens therein. In device 1800 of FIG. 18, the sidewall at the anterior portion of the device 1800 and the sidewall at the posterior of the device 1800 may form a sidewall angle 1824, formed at a slot of the device 1800. For example, the sidewall angle may be about 69°. Furthermore, device 1800 may not comprise any ribs, such as ribs 105 or ribs 305. Instead, device 1800 may comprise a slot within the device cavity configured to secure an intraocular lens therein.

In some embodiments, a length of a major axis of the device 1800 or a length measured from the outermost end of one sidewall to the outermost end of another sidewall along a major axis of the device 1800 can be about 9.65 mm. In other embodiments, the length of the major axis of the device 1800 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 1800 may comprise a diameter 1802 of the device 1800.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 1804, which can be a diameter of an anterior opening and/or a posterior opening.

In some embodiments, the taper length 1814 may comprise a distance between the exterior surface of the sidewall at its most anterior/posterior point and the exterior surface at the anterior/posterior opening. In some embodiments, the exterior surface may comprise curved surfaces 1822 and 1816 at the slot and the openings, respectively. The curved shape of the sidewalls may contribute to a reduction in post-surgical complications through minimization of contact or the severity of contact between the device 1800 and the iris. The curved shape of the sidewall of device 1800 near the openings is shown in Detail B.

In some embodiments, the device 1800 may comprise an inner diameter 1806 comprising the distance between the interior surfaces of the sidewalls at the ridge. In some embodiments, the inner diameter 1806 may be about 9.15 mm. In some embodiments, the interior diameter may be between about 5.00 mm and 15.00 mm.

In some embodiments, the device 1800 may comprise a slot thickness 1812 comprising the size of the slot. In some embodiments, the slot thickness 1812 may be about 0.40 mm. In some embodiments, the slot thickness 1812 may be between about 0.10 mm and about 1.00 mm. In some embodiments, the slot thickness may be configured to reduce the possibility of lens tilt by an intraocular lens located in the slot.

In some embodiments, a thickness 1808 of the device 1800 may comprise a maximum distance between the anterior side and posterior side of the device 1800. In some embodiments, the thickness 1808 of the device 1800 may be about 3.00 mm. In some embodiments, the thickness 1808 of the device 1800 may be between about 0.5 mm and 4.0 mm. In some embodiments, the thickness 1808 of the device 1800 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

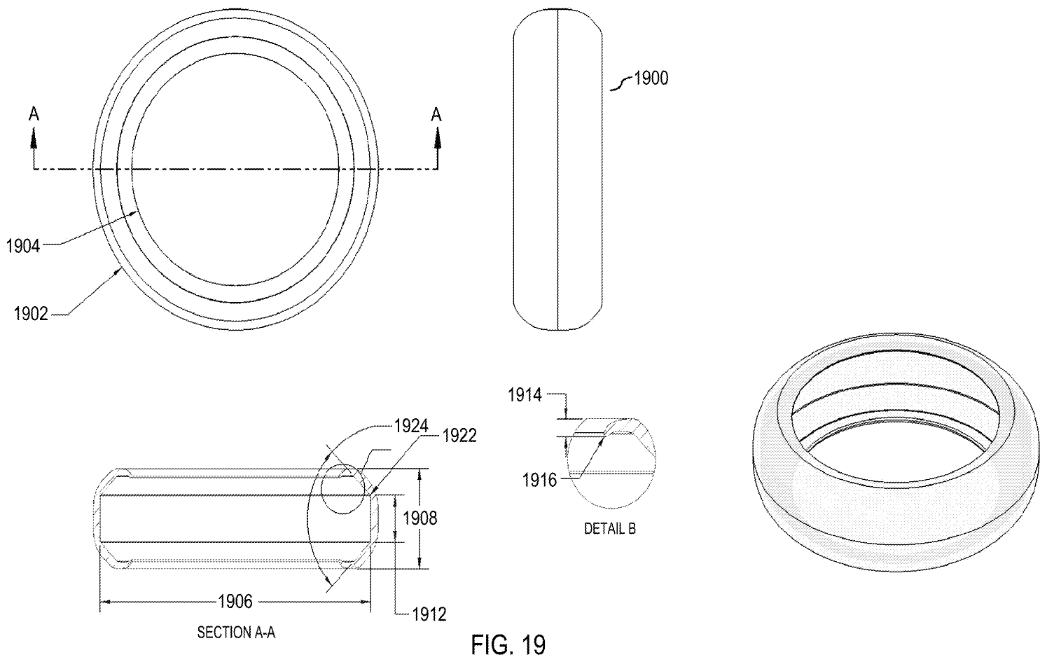

FIG. 19 illustrates another example prosthetic capsular device according to some embodiments herein. In device 1900 of FIG. 19, the sidewall at the anterior portion of the device 1900 and the sidewall at the posterior of the device 1900 may form a slot of the device 1900. In some embodiments, the device 1900 may comprise a cylindrical middle portion perpendicular to the anterior opening and the posterior opening. Device 1900 may comprise a slot within the device cavity configured to secure an intraocular lens therein. In device 1900 of FIG. 19, the sidewall at the anterior portion of the device 1900 and the sidewall at the posterior of the device 1900 may form a sidewall angle 1924, formed at a slot of the device 1900. For example, the sidewall angle may be about 91°. Furthermore, device 1900 may not comprise any ribs, such as ribs 105 or ribs 305. Instead, device 1900 may comprise a slot within the device cavity configured to secure an intraocular lens therein.

In some embodiments, a length of a major axis of the device 1900 or a length measured from the outermost end of one sidewall to the outermost end of another sidewall along a major axis of the device 1900 can be about 9.65 mm. In other embodiments, the length of the major axis of the device 1900 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 1900 may comprise a diameter 1902 of the device 1900.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 1904, which can be a diameter of an anterior opening and/or a posterior opening.

In some embodiments, the taper length 1914 may comprise a distance between the exterior surface of the sidewall at its most anterior/posterior point and the exterior surface at the anterior/posterior opening. In some embodiments, the exterior surface may comprise curved surfaces 1922 and 1916 at the slot and the openings, respectively. The curved shape of the sidewalls may contribute to a reduction in post-surgical complications through minimization of contact or the severity of contact between the device 1900 and the iris. The curved shape of the sidewall of device 1900 near the openings is shown in Detail B.

In some embodiments, the device 1900 may comprise an inner diameter 1906 comprising the distance between the interior surfaces of the sidewalls at the ridge. In some embodiments, the inner diameter 1906 may be about 9.15 mm. In some embodiments, the interior diameter may be between about 5.00 mm and 15.00 mm.

In some embodiments, the device 1900 may comprise a slot thickness 1912 comprising the size of the slot. In some embodiments, the slot thickness 1912 may be about 1.40 mm. In some embodiments, the slot thickness 1912 may be between about 0.10 mm and about 2.00 mm. In some embodiments, the slot thickness may be configured to reduce the possibility of lens tilt by an intraocular lens located in the slot.

In some embodiments, a thickness 1908 of the device 1900 may comprise a maximum distance between the anterior side and posterior side of the device 1900. In some embodiments, the thickness 1908 of the device 1900 may be about 3.00 mm. In some embodiments, the thickness 1908 of the device 1900 may be between about 0.5 mm and 4.0 mm. In some embodiments, the thickness 1908 of the device 1900 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

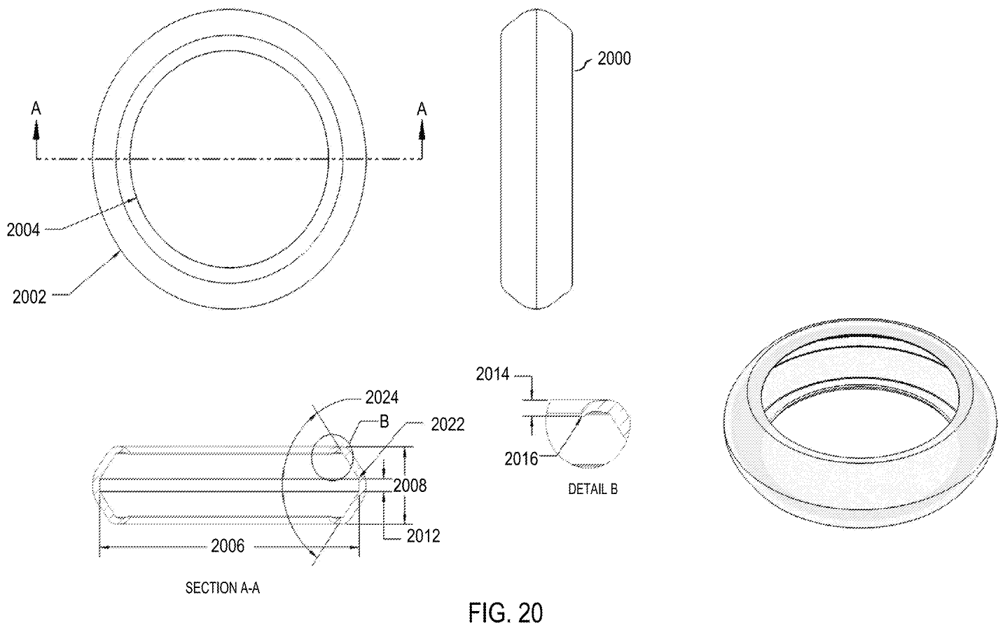

FIG. 20 illustrates another example prosthetic capsular device according to some embodiments herein. In device 2000 of FIG. 20, the sidewall at the anterior portion of the device 2000 and the sidewall at the posterior of the device 2000 may form a slot of the device 2000. In some embodiments, device 2000 may comprise a cylindrical middle portion perpendicular to the anterior opening and the posterior opening. Device 2000 may comprise a slot within the device cavity configured to secure an intraocular lens therein. In device 2000 of FIG. 20, the sidewall at the anterior portion of the device 2000 and the sidewall at the posterior of the device 2000 may form a sidewall angle 2024, formed at a slot of the device 2000. For example, the sidewall angle may be about 1110. Furthermore, device 2000 may not comprise any ribs, such as ribs 105 or ribs 305. Instead, device 2000 may comprise a slot within the device cavity configured to secure an intraocular lens therein.

In some embodiments, a length of a major axis of the device 2000 or a length measured from the outermost end of one sidewall to the outermost end of another sidewall along a major axis of the device 2000 can be about 9.65 mm. In other embodiments, the length of the major axis of the device 2000 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 2000 may comprise a diameter 2002 of the device 2000.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 2004, which can be a diameter of an anterior opening and/or a posterior opening.

In some embodiments, the taper length 2014 may comprise a distance between the exterior surface of the sidewall at its most anterior/posterior point and the exterior surface at the anterior/posterior opening. In some embodiments, the exterior surface may comprise curved surfaces 2022 and 2016 at the slot and the openings, respectively. The curved shape of the sidewalls may contribute to a reduction in post-surgical complications through minimization of contact or the severity of contact between the device 2000 and the iris. The curved shape of the sidewall of the device 2000 near the openings is shown in Detail B.

In some embodiments, the device 2000 may comprise an inner diameter 2006 comprising the distance between the interior surfaces of the sidewalls at the ridge. In some embodiments, the inner diameter 2006 may be about 9.15 mm. In some embodiments, the interior diameter may be between about 5.00 mm and 15.00 mm.

In some embodiments, the device 2000 may comprise a slot thickness 2012 comprising the size of the slot. In some embodiments, the slot thickness 2012 may be about 0.40 mm. In some embodiments, the slot thickness 2012 may be between about 0.10 mm and about 2.00 mm. In some embodiments, the slot thickness may be configured to reduce the possibility of lens tilt by an intraocular lens located in the slot.

In some embodiments, a thickness 2008 of the device 2000 may comprise a maximum distance between the anterior side and posterior side of the device 2000. In some embodiments, the thickness 2008 of the device 2000 may be about 2.50 mm. In some embodiments, the thickness 2008 of the device 2000 may be between about 0.5 mm and 4.0 mm. In some embodiments, the thickness 2008 of the device 2000 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

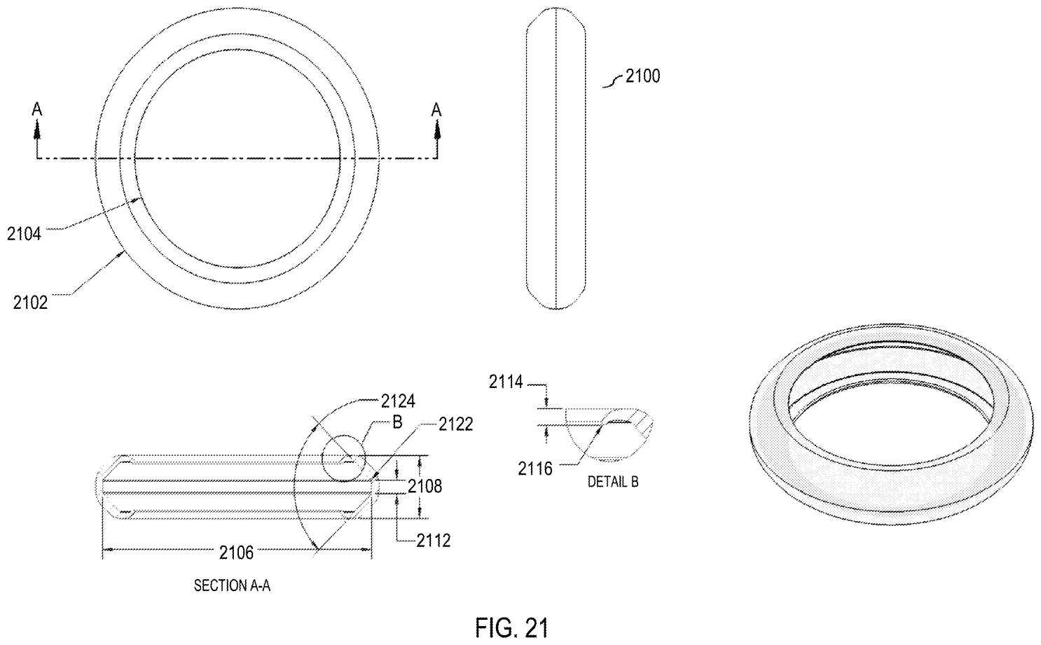

FIG. 21 illustrates another example prosthetic capsular device according to some embodiments herein. In device 2100 of FIG. 21, the sidewall at the anterior portion of device 2100 and the sidewall at the posterior of the device 2100 may form a slot of the device 2100. In some embodiments, device 2100 may comprise a cylindrical middle portion perpendicular to the anterior opening and the posterior opening. Device 2100 may comprise a slot within the device cavity configured to secure an intraocular lens therein. In device 2100 of FIG. 21, the sidewall at the anterior portion of device 2100 and the sidewall at the posterior of the device 2100 may form a sidewall angle 2124, formed at a slot of the device 2100. For example, the sidewall angle may be about 91°. Furthermore, device 2100 may not comprise any ribs, such as ribs 105 or ribs 305. Instead, device 2100 may comprise a slot within the device cavity configured to secure an intraocular lens therein.

In some embodiments, a length of a major axis of the device 2100 or a length measured from the outermost end of one sidewall to the outermost end of another sidewall along a major axis of the device 2100 can be about 9.65 mm. In other embodiments, the length of the major axis of the device 2100 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 2100 may comprise a diameter 2102 of the device 2100.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 2104, which can be a diameter of an anterior opening and/or a posterior opening.

In some embodiments, the taper length 2114 may comprise a distance between the exterior surface of the sidewall at its most anterior/posterior point and the exterior surface at the anterior/posterior opening. In some embodiments, the exterior surface may comprise curved surfaces 2122 and 2116 at the slot and the openings, respectively. The curved shape of the sidewalls may contribute to a reduction in post-surgical complications through minimization of contact or the severity of contact between device 2100 and the iris. The curved shape of the sidewall of device 2100 near the openings is shown in Detail B.

In some embodiments, device 2100 may comprise an inner diameter 2106 comprising the distance between the interior surfaces of the sidewalls at the ridge. In some embodiments, the inner diameter 2106 may be about 9.15 mm. In some embodiments, the interior diameter may be between about 5.00 mm and 15.00 mm.

In some embodiments, device 2100 may comprise a slot thickness 2112 comprising the size of the slot. In some embodiments, the slot thickness 2112 may be about 0.40 mm. In some embodiments, the slot thickness 2112 may between about 0.10 mm and about 2.00 mm. In some embodiments, the slot thickness may be configured to reduce the possibility of lens tilt by an intraocular lens located in the slot.

In some embodiments, a thickness 2108 of device 2100 may comprise a maximum distance between the anterior side and posterior side of the device 2100. In some embodiments, the thickness 2108 of device 2100 may be about 2.00 mm. In some embodiments, the thickness 2108 of device 2100 may be between about 0.5 mm and 4.0 mm. In some embodiments, the thickness 2108 of the device 2100 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

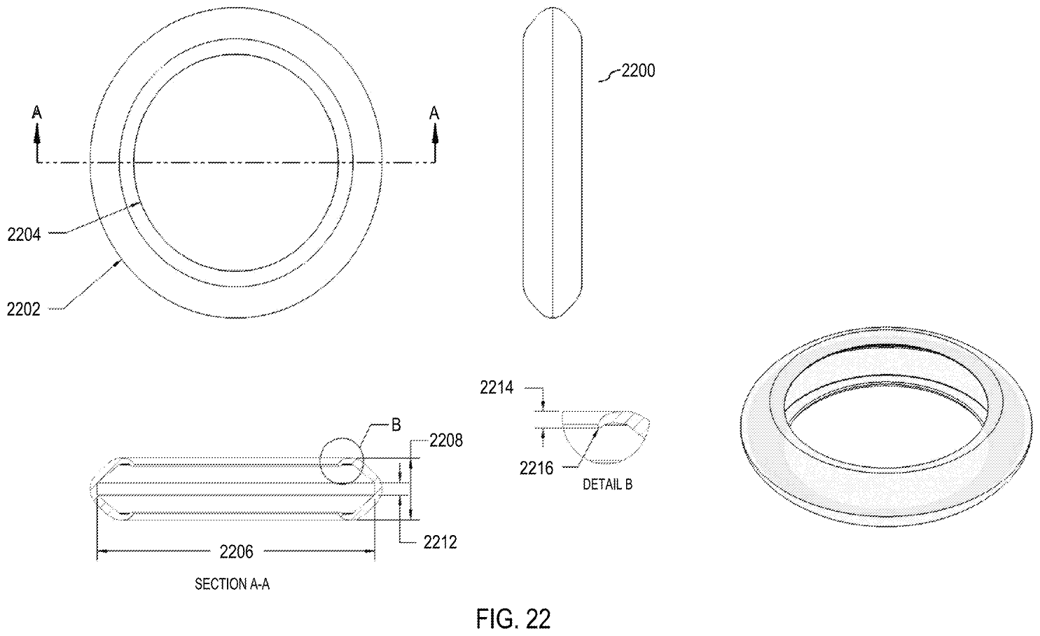

FIG. 22 illustrates another example prosthetic capsular device according to some embodiments herein. In device 2200 of FIG. 22, the sidewall at the anterior portion of device 2200 and the sidewall at the posterior of device 2200 may form a slot of device 2200. In some embodiments, device 2200 may comprise a cylindrical middle portion perpendicular to the anterior opening and the posterior opening. Device 2200 may comprise a slot within the device cavity configured to secure an intraocular lens therein.

In some embodiments, a length of a major axis of the device 2200 or a length measured from the outermost end of one sidewall to the outermost end of another sidewall along a major axis of the device 2200 can be about 10.00 mm. In other embodiments, the length of the major axis of the device 2200 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 2200 may comprise a diameter 2202 of the device 2200.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 2204, which can be a diameter of an anterior opening and/or a posterior opening.

In some embodiments, the taper length 2214 may comprise a distance between the exterior surface of the sidewall at its most anterior/posterior point and the exterior surface at the anterior/posterior opening. In some embodiments, the exterior surface may comprise curved surfaces 2216 at the openings. The curved shape of the sidewalls may contribute to a reduction in post-surgical complications through minimization of contact or the severity of contact between device 2200 and the iris. The curved shape of the sidewall of device 2200 near the openings is shown in Detail B.

In some embodiments, device 2200 may comprise an inner diameter 2206 comprising the distance between the interior surfaces of the sidewalls at the ridge. In some embodiments, the inner diameter 2206 may be about 9.50 mm. In some embodiments, the interior diameter may be between about 5.00 mm and 15.00 mm.

In some embodiments, device 2200 may comprise a slot thickness 2212 comprising the size of the slot. In some embodiments, the slot thickness 2212 may be about 0.40 mm. In some embodiments, the slot thickness 2212 may be between about 0.10 mm and about 2.00 mm. In some embodiments, the slot thickness may be configured to reduce the possibility of lens tilt by an intraocular lens located in the slot.

In some embodiments, a thickness 2208 of the device 2200 may comprise a maximum distance between the anterior side and posterior side of the device 2200. In some embodiments, the thickness 2208 of device 2200 may be about 2.00 mm. In some embodiments, the thickness 2208 of device 2200 may be between about 0.5 mm and 4.0 mm. In some embodiments, the thickness 2208 of the device 2200 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

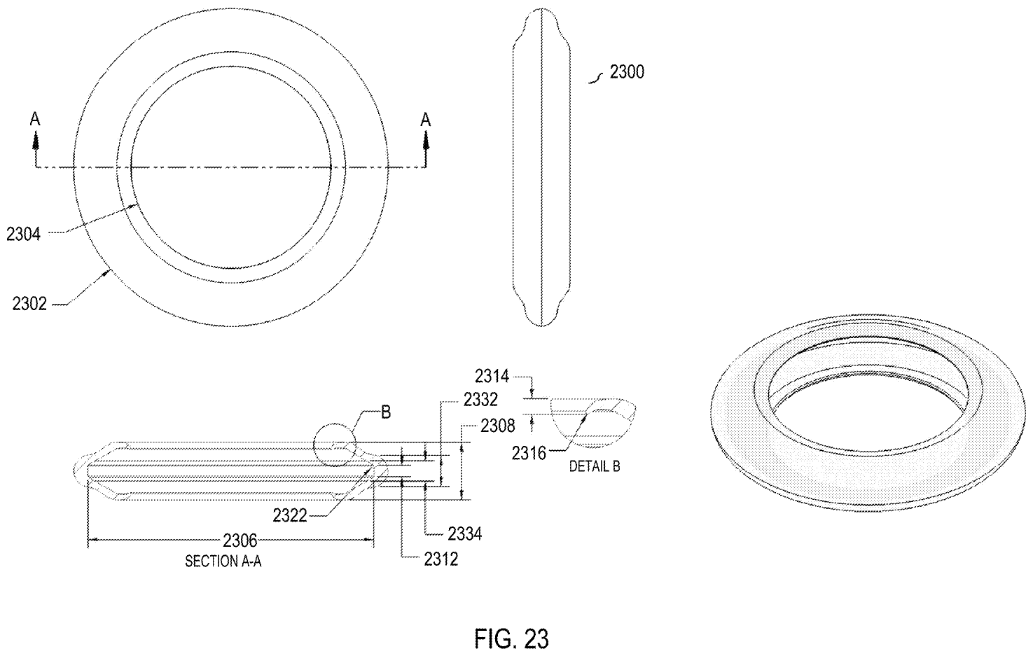

FIG. 23 illustrates another example prosthetic capsular device according to some embodiments herein. In device 2300 of FIG. 23, the sidewall at the anterior portion of the device 2300 and the sidewall at the posterior of the device 2300 may form a slot of the device 2300. In some embodiments, the device 2300 may comprise a rounded middle portion protruding laterally outward from the anterior portion and the posterior portion. In some embodiments, the rounded middle portion may comprise a middle portion length 2332, measured from the exterior of the device. In some embodiments, the middle portion length 2332 may be about 1.08 mm. In some embodiments, the rounded middle portion may comprise a middle portion interior length 2334, measuring on the interior of the device. In some embodiments, the middle portion interior length 2334 may be about 0.70 mm. Device 2300 may comprise a slot within the device cavity configured to secure an intraocular lens therein.

In some embodiments, a length of a major axis of the device 2300 or a length measured from the outermost end of one sidewall to the outermost end of another sidewall along a major axis of the device 2300 can be about 11.00 mm. In other embodiments, the length of the major axis of the device 2300 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 2300 may comprise a diameter 2302 of the device 2300.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 2304, which can be a diameter of an anterior opening and/or a posterior opening.

In some embodiments, the taper length 2314 may comprise a distance between the exterior surface of the sidewall at its most anterior/posterior point and the exterior surface at the anterior/posterior opening. In some embodiments, the exterior surface may comprise curved surfaces 2322 and 2316 at the slot and the openings, respectively. The curved shape of the sidewalls may contribute to a reduction in post-surgical complications through minimization of contact or the severity of contact between the device 2300 and the iris. The curved shape of the sidewall of device 2300 near the openings is shown in Detail B.

In some embodiments, device 2300 may comprise an inner diameter 2306 comprising the distance between the interior surfaces of the sidewalls at the ridge. In some embodiments, the inner diameter 2306 may be about 10.00 mm. In some embodiments, the interior diameter may be between about 5.00 mm and 15.00 mm.

In some embodiments, device 2300 may comprise a slot thickness 2312 comprising the size of the slot. In some embodiments, the slot thickness 2312 may be about 0.40 mm. In some embodiments, the slot thickness 2312 may be between about 0.10 mm and about 2.00 mm. In some embodiments, the slot thickness may be configured to reduce the possibility of lens tilt by an intraocular lens located in the slot.

In some embodiments, a thickness 2308 of the device 2300 may comprise a maximum distance between the anterior side and posterior side of the device 2300. In some embodiments, the thickness 2308 of device 2300 may be about 2.00 mm. In some embodiments, the thickness 2308 of the device 2300 may be between about 0.5 mm and 4.0 mm. In some embodiments, the thickness 2308 of the device 2300 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

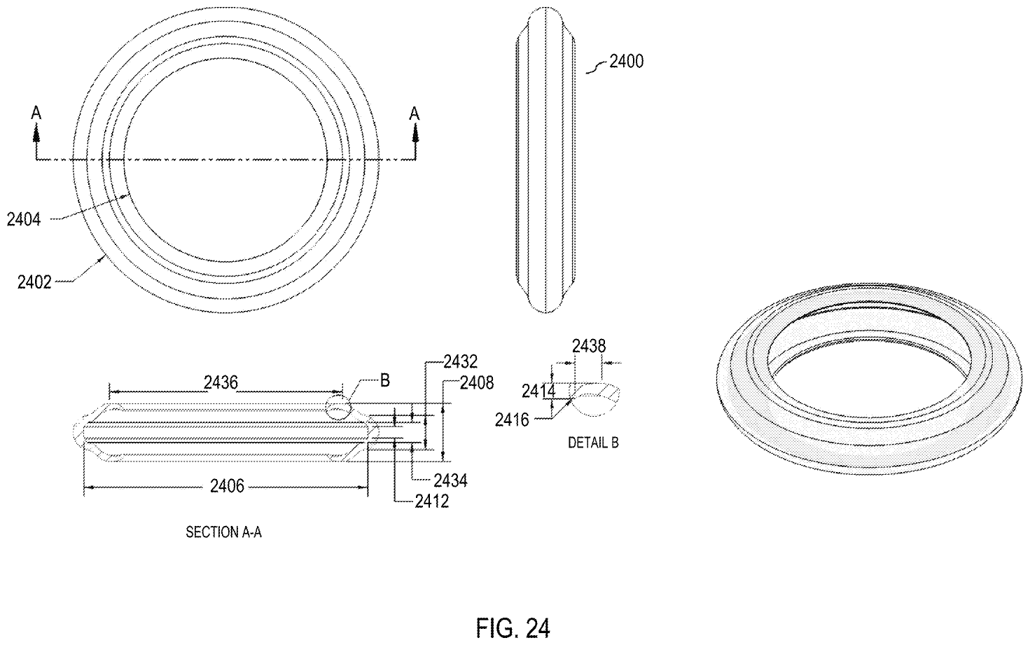

FIG. 24 illustrates another example prosthetic capsular device according to some embodiments herein. In device 2400 of FIG. 24, the sidewall at the anterior portion of the device 2400 and the sidewall at the posterior of the device 2400 may form a slot of the device 2400. In some embodiments, device 2400 may comprise a rounded middle portion protruding laterally outward from the anterior portion and the posterior portion. In some embodiments, the rounded middle portion may comprise a middle portion length 2432, measured from the exterior of the device. In some embodiments, the middle portion length 2432 may be about 1.17 mm. In some embodiments, the rounded middle portion may comprise a middle portion interior length 2434, measuring on the interior of the device. In some embodiments, the middle portion interior length 2434 may be about 0.70 mm. Device 2400 may comprise a slot within the device cavity configured to secure an intraocular lens therein.

In some embodiments, a length of a major axis of the device 2400 or a length measured from the outermost end of one sidewall to the outermost end of another sidewall along a major axis of the device 2400 can be about 10.50 mm. In other embodiments, the length of the major axis of the device 2400 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 2400 may comprise a diameter 2402 of the device 2400.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 2404, which can be a diameter of an anterior opening and/or a posterior opening. In some embodiments, the device may comprise a rim diameter 2436 comprising the diameter of the device formed by a rim surrounding the anterior opening and the posterior opening. In some embodiments, the rim diameter 2436 may measure between about 5.0 mm to about 15.0 mm. In some embodiments, the rim diameter 2436 may be about 8.0 mm. In some embodiments, the rim may comprise a rim length comprising a distance from the opening to a sidewall of the device. In some embodiments, the rim length may be about 0.1 mm to about 1.0 mm. In some embodiments, the rim length may be about 0.46 mm.

In some embodiments, the taper length 2414 may comprise a distance between the exterior surface of the sidewall at its most anterior/posterior point and the exterior surface at the anterior/posterior opening. In some embodiments, the exterior surface may comprise curved surfaces 2422 and 2416 at the slot and the openings, respectively. The curved shape of the sidewalls may contribute to a reduction in post-surgical complications through minimization of contact or the severity of contact between the device 2400 and the iris. The curved shape of the sidewall of device 2400 near the openings is shown in Detail B.

In some embodiments, the device 2400 may comprise an inner diameter 2406 comprising the distance between the interior surfaces of the sidewalls at the ridge. In some embodiments, the inner diameter 2406 may be about 9.75 mm. In some embodiments, the interior diameter may be between about 5.00 mm and 15.00 mm.

In some embodiments, the device 2400 may comprise a slot thickness 2412 comprising the size of the slot. In some embodiments, the slot thickness 2412 may be about 0.40 mm. In some embodiments, the slot thickness 2412 may be between about 0.10 mm and about 2.00 mm. In some embodiments, the slot thickness may be configured to reduce the possibility of lens tilt by an intraocular lens located in the slot.

In some embodiments, a thickness 2408 of the device 2400 may comprise a maximum distance between the anterior side and posterior side of the device 2400. In some embodiments, the thickness 2408 of the device 2400 may be about 2.00 mm. In some embodiments, the thickness 2408 of the device 2400 may be between about 0.5 mm and 4.0 mm. In some embodiments, the thickness 2408 of the device 2400 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

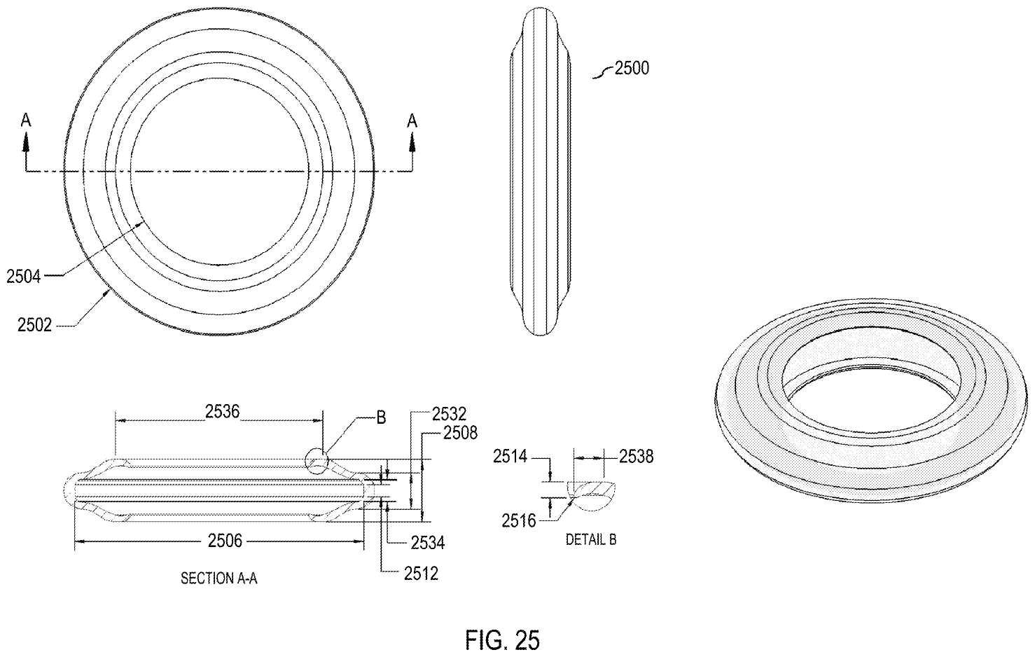

FIG. 25 illustrates another example prosthetic capsular device according to some embodiments herein. In device 2500 of FIG. 25, the sidewall at the anterior portion of device 2500 and the sidewall at the posterior of device 2500 may form a slot of device 2500. In some embodiments, device 2500 may comprise a rounded middle portion protruding laterally outward from the anterior portion and the posterior portion. In some embodiments, the rounded middle portion may comprise a middle portion length 2532, measured from the exterior of the device. In some embodiments, the middle portion length 2532 may be about 1.17 mm. In some embodiments, the rounded middle portion may comprise a middle portion interior length 2534, measuring on the interior of the device. In some embodiments, the middle portion interior length 2534 may be about 0.70 mm. Device 2500 may comprise a slot within the device cavity configured to secure an intraocular lens therein.

In some embodiments, a length of a major axis of the device 2500 or a length measured from the outermost end of one sidewall to the outermost end of another sidewall along a major axis of the device 2500 can be about 10.50 mm. In other embodiments, the length of the major axis of the device 2500 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 2500 may comprise a diameter 2502 of the device 2500.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 2504, which can be a diameter of an anterior opening and/or a posterior opening. In some embodiments, the device may comprise a rim diameter 2536 comprising the diameter of the device formed by a rim surrounding the anterior opening and the posterior opening. In some embodiments, the rim diameter 2536 may measure between about 5.0 mm to about 15.0 mm. In some embodiments, the rim diameter 2536 may be about 7.0 mm. In some embodiments, the rim may comprise a rim length comprising a distance from the opening to a sidewall of the device. In some embodiments, the rim length may be about 0.1 mm to about 1.0 mm. In some embodiments, the rim length may be about 0.50 mm.

In some embodiments, the taper length 2514 may comprise a distance between the exterior surface of the sidewall at its most anterior/posterior point and the exterior surface at the anterior/posterior opening. In some embodiments, the exterior surface may comprise curved surfaces 2516 at the openings. The curved shape of the sidewalls may contribute to a reduction in post-surgical complications through minimization of contact or the severity of contact between the device 2500 and the iris. The curved shape of the sidewall of device 2500 near the openings is shown in Detail B.

In some embodiments, device 2500 may comprise an inner diameter 2506 comprising the distance between the interior surfaces of the sidewalls at the ridge. In some embodiments, the inner diameter 2506 may be about 9.75 mm. In some embodiments, the interior diameter may be between about 5.00 mm and 15.00 mm.

In some embodiments, device 2500 may comprise a slot thickness 2512 comprising the size of the slot. In some embodiments, the slot thickness 2512 may be about 0.40 mm. In some embodiments, the slot thickness 2512 may be between about 0.10 mm and about 2.00 mm. In some embodiments, the slot thickness may be configured to reduce the possibility of lens tilt by an intraocular lens located in the slot.

In some embodiments, a thickness 2508 of the device 2500 may comprise a maximum distance between the anterior side and posterior side of the device 2500. In some embodiments, the thickness 2508 of the device 2500 may be about 2.00 mm. In some embodiments, the thickness 2508 of the device 2500 may be between about 0.5 mm and 4.0 mm. In some embodiments, the thickness 2508 of the device 2500 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

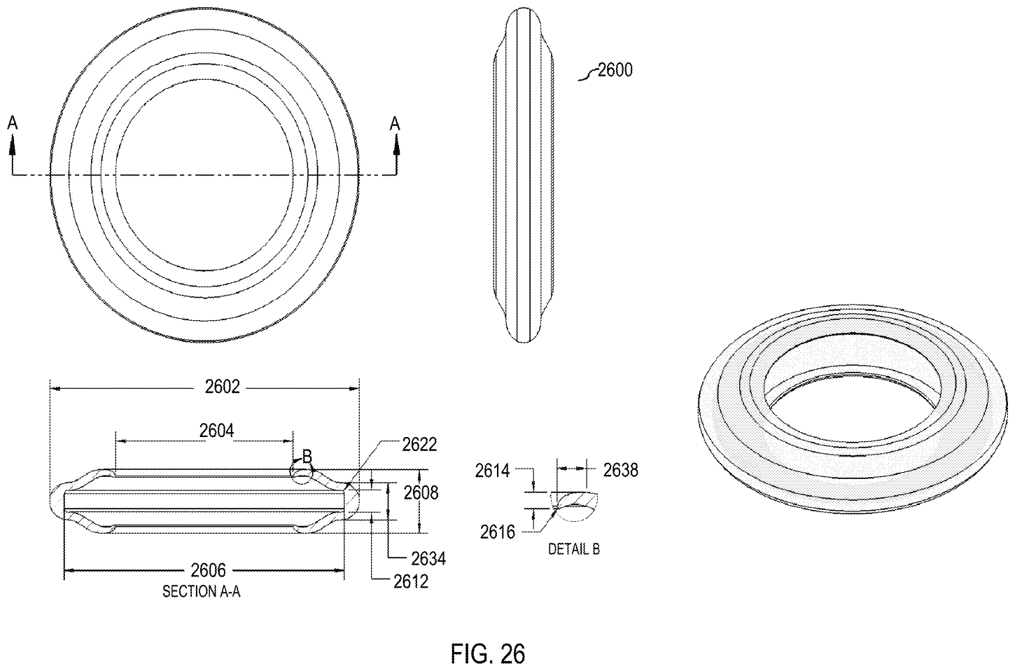

FIG. 26 illustrates another example prosthetic capsular device according to some embodiments herein. In device 2600 of FIG. 26, the sidewall at the anterior portion of device 2600 and the sidewall at the posterior of the device 2600 may form a slot of device 2600. In some embodiments, device 2600 may comprise a rounded middle portion protruding laterally outward from the anterior portion and the posterior portion. In some embodiments, the rounded middle portion may comprise a middle portion interior length 2634, measuring on the interior of the device. In some embodiments, the middle portion interior length 2634 may be about 0.70 mm. Device 2600 may comprise a slot within the device cavity configured to secure an intraocular lens therein.

In some embodiments, a length of a major axis of the device 2600 or a length measured from the outermost end of one sidewall to the outermost end of another sidewall along a major axis of the device 2600 can be about 10.50 mm. In other embodiments, the length of the major axis of the device 2600 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 2600 may comprise a diameter 2602 of the device 2600.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 2604, which can be a diameter of an anterior opening and/or a posterior opening.

In some embodiments, the taper length 2614 may comprise a distance between the exterior surface of the sidewall at its most anterior/posterior point and the exterior surface at the anterior/posterior opening. In some embodiments, the exterior surface may comprise curved surfaces 2616 at the openings. The curved shape of the sidewalls may contribute to a reduction in post-surgical complications through minimization of contact or the severity of contact between the device 2600 and the iris. The curved shape of the sidewall of device 2600 near the openings is shown in Detail B.

In some embodiments, device 2600 may comprise an inner diameter 2606 comprising the distance between the interior surfaces of the sidewalls at the ridge. In some embodiments, the inner diameter 2606 may be about 9.50 mm. In some embodiments, the interior diameter may be between about 5.00 mm and 15.00 mm.

In some embodiments, the device 2600 may comprise a slot thickness 2612 comprising the size of the slot. In some embodiments, the slot thickness 2612 may be about 0.70 mm. In some embodiments, the slot thickness 2612 may be between about 0.10 mm and about 2.00 mm. In some embodiments, the slot thickness may be configured to reduce the possibility of lens tilt by an intraocular lens located in the slot.

In some embodiments, a thickness 2608 of the device 2600 may comprise a maximum distance between the anterior side and posterior side of the device 2600. In some embodiments, the thickness 2608 of the device 2600 may be about 2.00 mm. In some embodiments, the thickness 2608 of device 2600 may be between about 0.5 mm and 4.0 mm. In some embodiments, the thickness 2608 of the device 2600 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

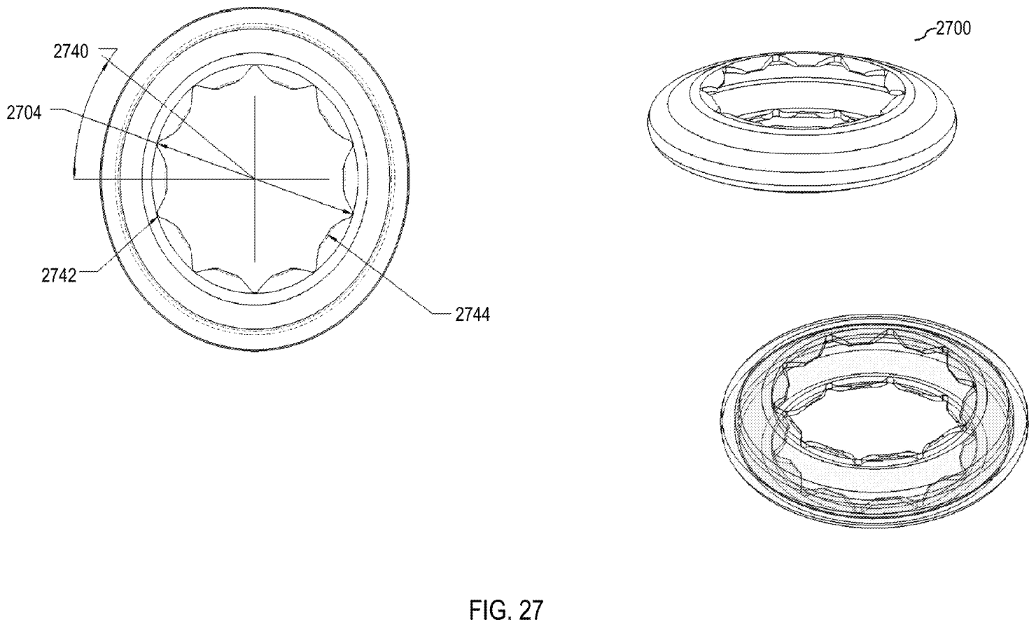

FIG. 27 illustrates another example prosthetic capsular device according to some embodiments herein. In device 2700 of FIG. 27, the sidewall at the anterior portion of the device 2700 and the sidewall at the posterior of the device 2700 may comprise one or more cutouts 2742, opening the anterior portion and the posterior portion of the device 2700 to the interior cavity. In some embodiments, there may be 10 cutouts in device 2700. However, the number and shape of the cutouts is not limited. In some embodiments, the cutouts 2742 may facilitate folding and expansion of the device or may allow for insertion of differently shaped or sized intraocular lenses.

In some embodiments, the cutouts 2742 may be substantially triangular with a rounded or blunted tip, wherein the tip comprises a radius. In some embodiments, the tip may comprise a radius of about 0.15 mm. In some embodiments, the tip may comprise a radius of about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, and/or within a range defined by two of the aforementioned values. In some embodiments, each cutout 2742 may extend around the circular opening in sectors of about 36°, although the angle may depend on the number of cutouts 2742. In some embodiments, the diameter 2704 of the cutouts 2742, measured at the tips of the cutouts, may be about 6.97 mm.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 2744, which can be a diameter of an anterior opening and/or a posterior opening.

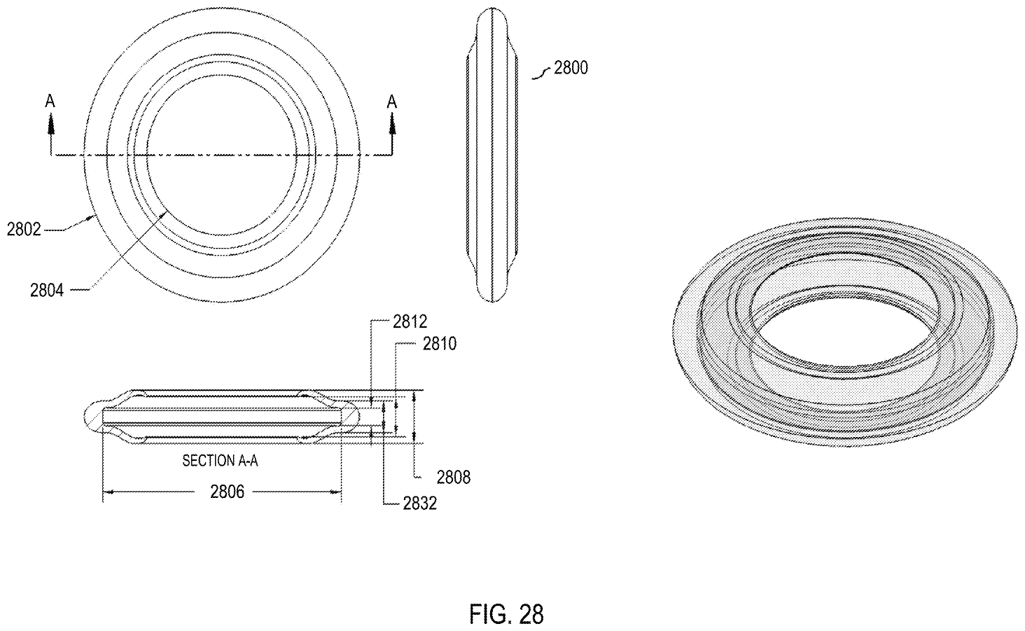

FIG. 28 illustrates another example prosthetic capsular device according to some embodiments herein. In device 2800 of FIG. 28, the sidewall at the anterior portion of the device 2800 and the sidewall at the posterior of the device 2800 may form a slot of the device 2800. In some embodiments, device 2800 may comprise a rounded middle portion protruding laterally outward from the anterior portion and the posterior portion. In some embodiments, the rounded middle portion may comprise a middle portion length 2832, measuring on the exterior of the device. In some embodiments, the middle portion length 2832 may be about 1.20 mm. Device 2800 may comprise a slot within the device cavity configured to secure an intraocular lens therein.

In some embodiments, a length of a major axis of the device 2800 or a length measured from the outermost end of one sidewall to the outermost end of another sidewall along a major axis of the device 2800 can be about 11.00 mm. In other embodiments, the length of the major axis of the device 2800 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 2800 may comprise a diameter 2802 of the device 2800.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 2804, which can be a diameter of an anterior opening and/or a posterior opening.

In some embodiments, device 2800 may comprise an inner diameter 2806 comprising the distance between the interior surfaces of the sidewalls at the ridge. In some embodiments, the inner diameter 2806 may be about 9.50 mm. In some embodiments, the interior diameter may be between about 5.00 mm and 15.00 mm.

In some embodiments, the device 2800 may comprise a slot thickness 2812 comprising the size of the slot. In some embodiments, the slot thickness 2812 may be about 0.67 mm. In some embodiments, the slot thickness 2812 may be between about 0.10 mm and about 2.00 mm. In some embodiments, the slot thickness may be configured to reduce the possibility of lens tilt by an intraocular lens located in the slot.

In some embodiments, a thickness 2808 of the device 2800 may comprise a maximum distance between the anterior side and posterior side of the device 2800. In some embodiments, the thickness 2808 of the device 2800 may be about 2.00 mm. In some embodiments, the thickness 2808 of the device 2800 may be between about 0.5 mm and 4.0 mm. In some embodiments, the thickness 2808 of the device 2800 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

In some embodiments, device 2800 may comprise an inner thickness 2810 comprising a distance between an inner surface of the sidewall at the anterior opening and an inner surface of the sidewall at the posterior opening. In some embodiments, the inner thickness 2810 may be about 1.48 mm.

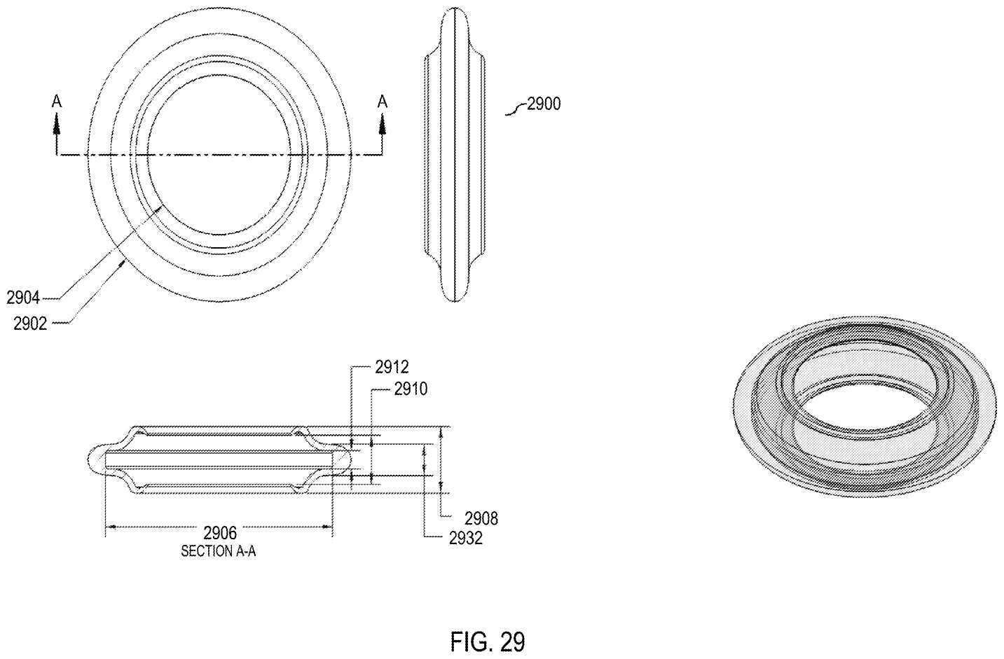

FIG. 29 illustrates another example prosthetic capsular device according to some embodiments herein. In device 2900 of FIG. 29, the sidewall at the anterior portion of device 2900 and the sidewall at the posterior of the device 2900 may form a slot of device 2900. In some embodiments, device 2900 may comprise a rounded middle portion protruding laterally outward from the anterior portion and the posterior portion. In some embodiments, the rounded middle portion may comprise a middle portion length 2932, measuring on the exterior of the device. In some embodiments, the middle portion length 2932 may be about 1.17 mm. Device 2900 may comprise a slot within the device cavity configured to secure an intraocular lens therein.

In some embodiments, a length of a major axis of the device 2900 or a length measured from the outermost end of one sidewall to the outermost end of another sidewall along a major axis of the device 2900 can be about 11.00 mm. In other embodiments, the length of the major axis of the device 2900 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 2900 may comprise a diameter 2902 of the device 2900.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 2904, which can be a diameter of an anterior opening and/or a posterior opening.

In some embodiments, device 2900 may comprise an inner diameter 2906 comprising the distance between the interior surfaces of the sidewalls at the ridge. In some embodiments, the inner diameter 2906 may be about 9.50 mm. In some embodiments, the interior diameter may be between about 5.00 mm and 15.00 mm.

In some embodiments, device 2900 may comprise a slot thickness 2912 comprising the size of the slot. In some embodiments, the slot thickness 2912 may be about 0.67 mm. In some embodiments, the slot thickness 2912 may be between about 0.10 mm and about 2.00 mm. In some embodiments, the slot thickness may be configured to reduce the possibility of lens tilt by an intraocular lens located in the slot.

In some embodiments, a thickness 2908 of the device 2900 may comprise a maximum distance between the anterior side and posterior side of the device 2900. In some embodiments, the thickness 2908 of device 2900 may be about 2.50 mm. In some embodiments, the thickness 2908 of the device 2900 may be between about 0.5 mm and 4.0 mm. In some embodiments, the thickness 2908 of the device 2900 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

In some embodiments, device 2900 may comprise an inner thickness 2910 comprising a distance between an inner surface of the sidewall at the anterior opening and an inner surface of the sidewall at the posterior opening. In some embodiments, the inner thickness 2910 may be about 1.85 mm.

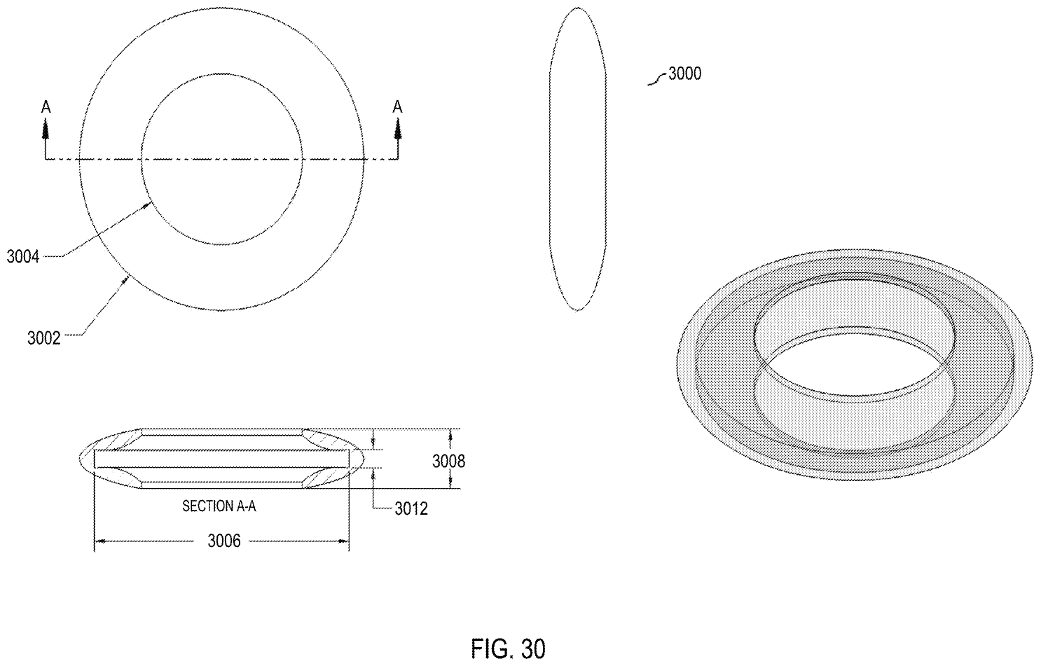

FIG. 30 illustrates another example prosthetic capsular device according to some embodiments herein. In device 3000 of FIG. 30, the sidewall at the anterior portion of device 3000 and the sidewall at the posterior of the device 3000 may form a slot of device 3000. In some embodiments, the device 3000 has a completely curved exterior surface, such that the device 3000 is disk-shaped.

In some embodiments, a length of a major axis of the device 3000 or a length measured from the outermost end of one sidewall to the outermost end of another sidewall along a major axis of the device 3000 can be about 11.50 mm. In other embodiments, the length of the major axis of the device 3000 can be about 5.00 mm, about 6.00 mm, about 7.00 mm, about 8.00 mm, about 9.00 mm, about 10.00 mm, about 11.00 mm, about 12.00 mm, about 13.00 mm, about 14.00 mm, about 15.00 mm, and/or within a range defined by two of the aforementioned values. In some embodiments, the length of the major axis of the device 3000 may comprise a diameter 3002 of device 3000.

In certain embodiments, the width of an opening of the cavity formed by each end of the two sidewalls can be about 5.00 mm. In certain embodiments, the width of an opening of the cavity formed by each end of the two sidewalls can be about 6.00 mm. In some embodiments, the width of the opening of the cavity formed by each end of the two sidewalls can be about 7.0 mm. In some embodiments, the width of the opening of the cavity formed by each end of the two sidewalls can be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, and/or within a range defined by two of the aforementioned values. In some embodiments, the width of the opening of the cavity formed by each end of the two sidewalls may comprise an opening diameter 3004, which can be a diameter of an anterior opening and/or a posterior opening.

In some embodiments, device 3000 may comprise an inner diameter 3006 comprising the distance between the interior surfaces of the sidewalls at the ridge. In some embodiments, the inner diameter 3006 may be about 10.30 mm. In some embodiments, the interior diameter may be between about 5.00 mm and 15.00 mm.

In some embodiments, device 3000 may comprise a slot thickness 3012 comprising the size of the slot. In some embodiments, the slot thickness 3012 may be about 0.70 mm. In some embodiments, the slot thickness 3012 may be between about 0.10 mm and about 2.00 mm. In some embodiments, the slot thickness may be configured to reduce the possibility of lens tilt by an intraocular lens located in the slot.

In some embodiments, a thickness 3008 of device 3000 may comprise a maximum distance between the anterior side and posterior side of the device 3000. In some embodiments, the thickness 3008 of the device 3000 may be about 2.27 mm. In some embodiments, the thickness 3008 of the device 3000 may be between about 0.5 mm and 4.0 mm. In some embodiments, the thickness 3008 of the device 3000 may about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, or any value between the aforementioned values.

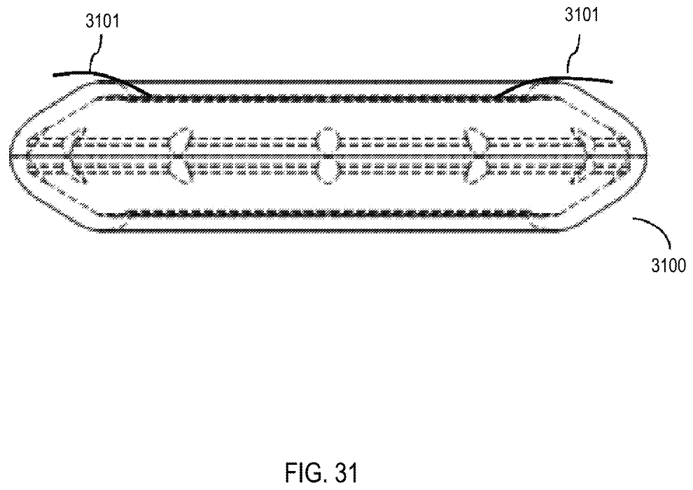

FIG. 31 illustrates an example of the interaction between the curved section of the sidewall and the iris of the eye. The curved section of the sidewalls may form a concavity in the sidewalls that reduces the amount and/or severity of contact between the device 3100 and posterior of the iris 3101. In some embodiments, the smoother contact between the device 3100 and the posterior of the iris 3101 may reduce or eliminate pigment dispersion and/or inflammation that can be caused using other devices. In some embodiments, at least a portion of an external surface of the sidewalls of the devices herein may be curved, while at least of portion of an interior surface of the sidewalls may be straight. In some embodiments, device 3100 may correspond to device 100 or any of the prosthetic capsular devices shown in the Figures. For example, the exterior surface of device 3100 may correspond to the exterior surface of device 100.

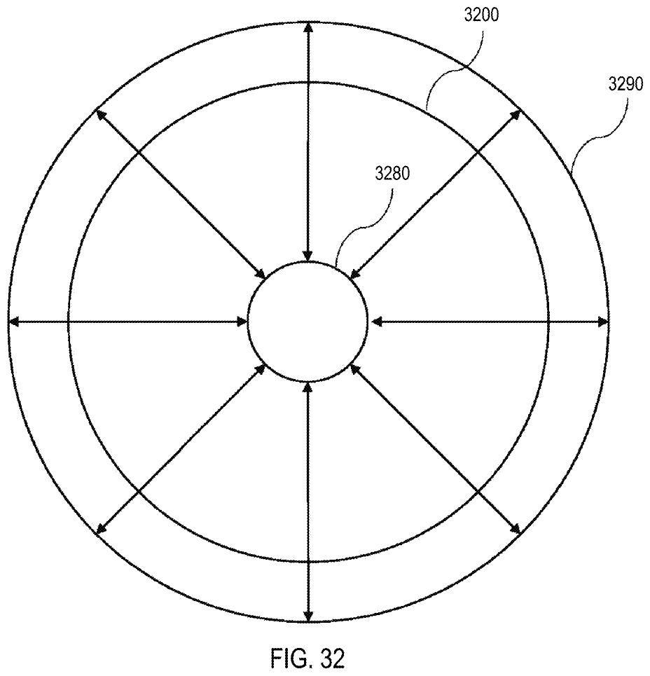

FIG. 32 illustrates an example diagram of a prosthetic capsular device within an eye. The prosthetic capsular device 3200 may comprise any of the devices described herein. The diagram illustrates an eye with a dilated pupil 3290 and with a contracted pupil 3280. As the pupil contracts and dilates when viewing objects, the posterior surface of the iris may contact device 3200. In some embodiments, the shape, size, and tapered surfaces of the devices described herein may reduce or mitigate the severity of the contact between the iris and the device. As a result, post-surgical complications may be minimized.

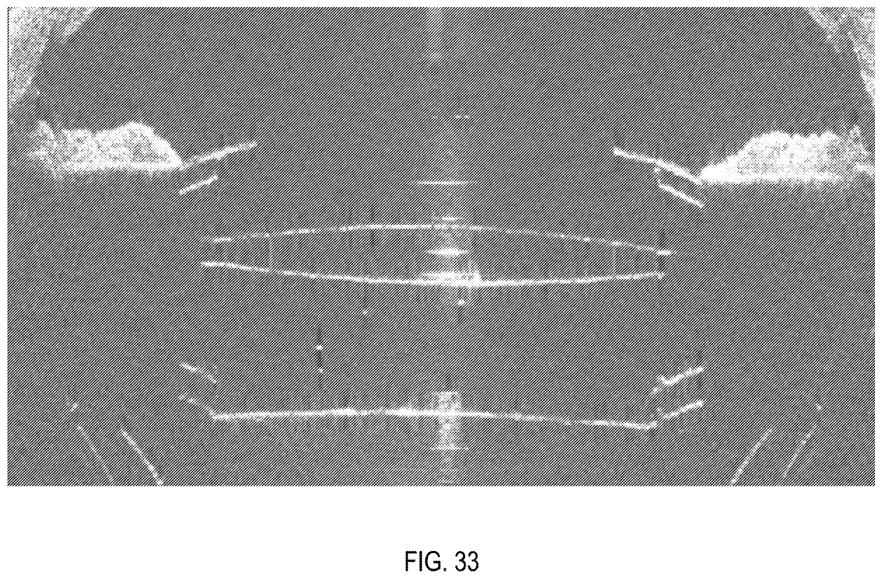

FIG. 33 illustrates an example image of a prosthetic capsular device within an eye. The prosthetic capsular device may comprise device 100 or any of the other prosthetic capsular devices shown in the Figures.

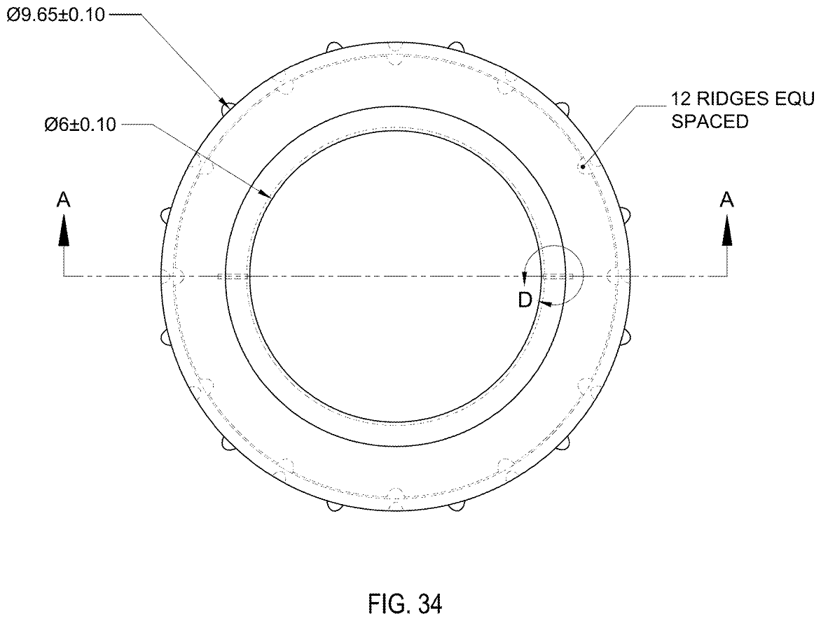

FIG. 34 illustrates another example prosthetic device according to some embodiments herein. The device of FIG. 34 may comprise some or all of the features described with respect to the devices of FIGS. 1-33. In some embodiments, the prosthetic capsular device may comprise one of more tabs, notches, or ribs on an interior surface of the device. In some embodiments, the one of more tabs, notches, or ribs on an interior surface of the device may project inwardly, into the central cavity of the device. In some embodiments, the one or more tabs, notches, or ribs on an interior surface of the device may be configured to couple, contact, join, or otherwise interact with an IOL or haptics of an IOL. In some embodiments, the one or more tabs, notches, or ribs on an interior surface of the device may prevent rotation or translation of an IOL within the device. Preventing rotation of an IOL within the device may be particularly important in cases in which a toric IOL is used, as the rotational position of the lens within the eye is significant to the functionality of the toric lens. The size, shape, location, and orientation of the one or more tabs, notches, or ribs on the interior surface of the device is not limited. Preferably, the one or more tabs, notches, or ribs on the interior surface of the device may be located such that they may couple, contact, join, or otherwise interact with an IOL or haptics of an IOL.

In some embodiments, in addition to the one or more tabs, notches, or ribs on an interior surface of the device, the prosthetic capsular device may comprise one or more tabs, notches, or ribs on an exterior surface of the device, protruding radially outward from the exterior surface. In some embodiments, the one or more tabs, notches, or ribs on an exterior surface of the device may contact or engage a surface of the natural capsular bag. In some embodiments, this contact of the one or more tabs, notches, or ribs with the natural capsular bag may prevent or eliminate rotation of the prosthetic capsular device within the eye. Again, preventing rotation may be particularly important in the case in which a toric IOL is inserted within the prosthetic capsular device. The size, shape, location, and orientation of the one or more tabs, notches, or ribs on the exterior surface of the device is not limited. However, in some embodiments, the one or more tabs, notches, or ribs on the exterior surface of the device may be located at a radially outward location, such as along a midpoint of the device along the longitudinal axis, such that the one or more tabs, notches, or ribs may contact the natural capsular bag.

FIG. 35 illustrates another example prosthetic device according to some embodiments herein. In some embodiments, in addition to, or as an alternative to, the one or more tabs, notches, or ribs on the exterior surface of the device, the prosthetic capsular device may comprise one or more ridges extending along at least a portion of the exterior surface of the device. For example, as illustrated in FIG. 35, one or more ridges may extend along the entirety of the device from an anterior opening to a posterior opening. However, in some embodiments, the ridges may extend only along a portion of the device in the longitudinal direction. Furthermore, although one or more ridges are illustrated as being present around the entirety of the circumference of the exterior sidewall of the device, the one or more ridges may be present around only a portion of the circumference. As with the one or more tabs, notches, or ribs on the exterior surface of the device, the one or more ridges may contact a surface of the natural capsular bag to prevent rotation and/or translation of the prosthetic capsular device within the eye. In some embodiments, in addition to, or as an alternative to, the one or more tabs, notches, ribs, or ridges on the exterior surface of the device, the exterior surface of the device may comprise one or more textured surfaces. In some embodiments, a textured surface may provide enhanced grip or friction with the natural capsular bag, which may prevent or reduce translational or rotational movement of the device within the eye. In some embodiments, the textured surface may comprise an adhesive, nanostructures or micro-structures formed on the exterior surface, a separate material formed on the exterior surface, or may be formed using other methods known in the art.

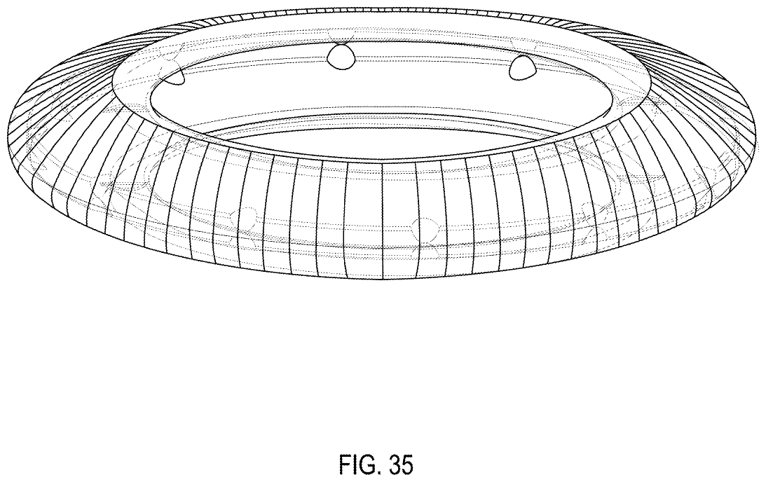

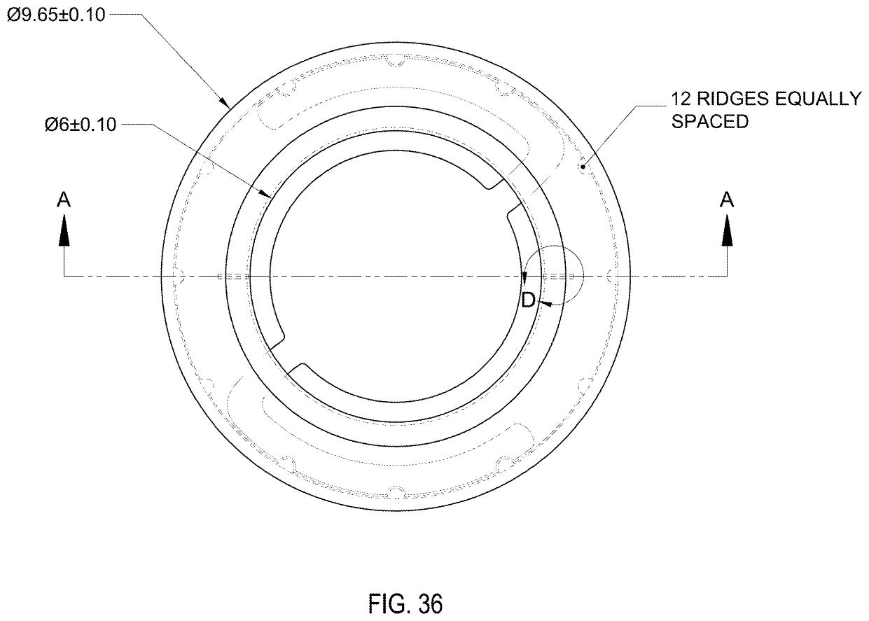

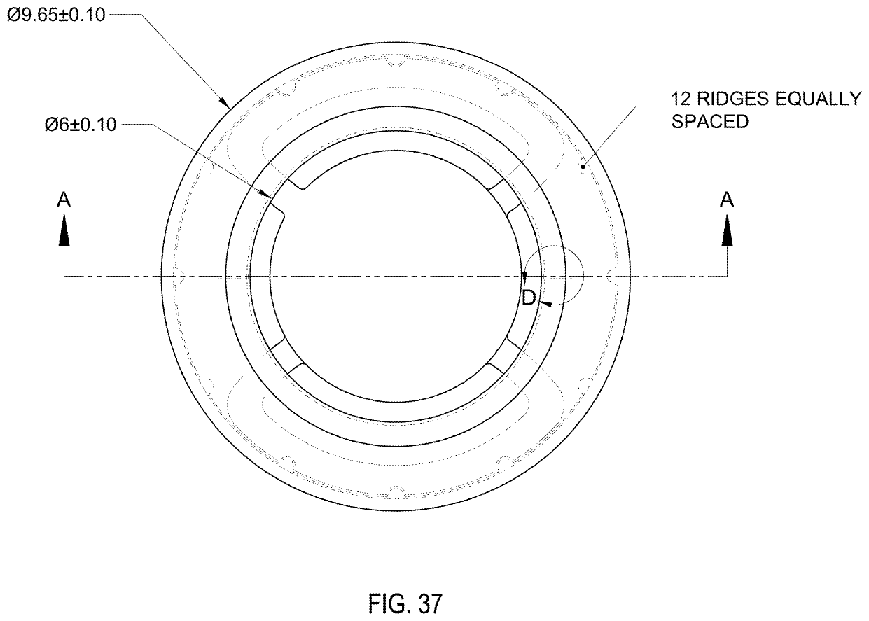

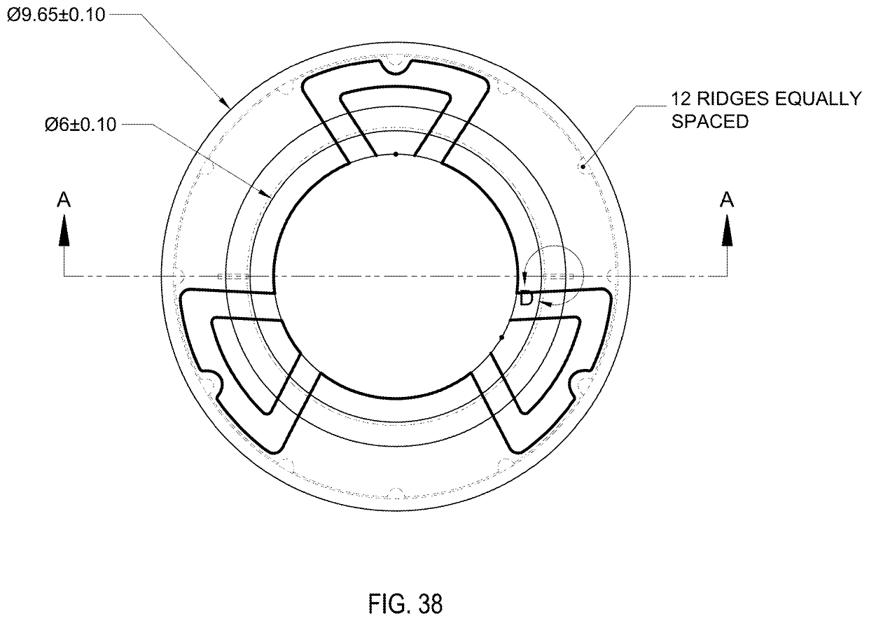

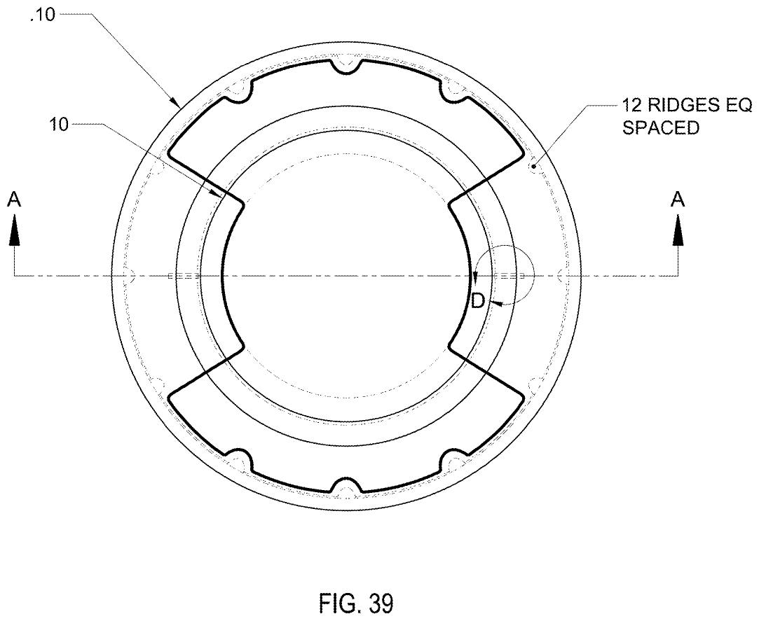

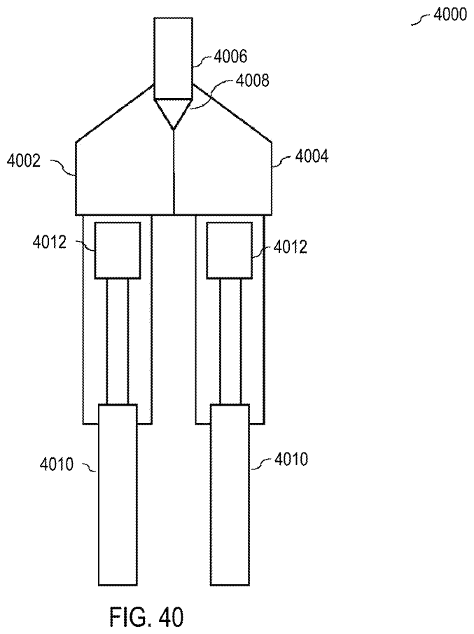

FIG. 36 illustrates another example prosthetic device and an example intraocular lens therein according to some embodiments herein. In particular, FIG. 36 illustrates a prosthetic implant device with an IOL implanted therein, wherein haptics of the IOL lock together with one or more tabs, notches, or ribs on an interior surface of the device within the cavity of the capsule. In some embodiments, one or more notches in the haptic or haptics of the IOL may couple, contact, join, or otherwise interact with the one or more tabs, notches, or ribs on an interior surface of the device. The shape, size, location, and orientation of IOLs and haptics that may be coupled to the one or more tabs, notches, or ribs on an interior surface of the device is not particularly limited. For the sake of example, additional IOLs and haptic shapes are illustrated in FIGS. 37-39. In some embodiments, disclosed herein are one or more “lock and key” kit configurations comprising a prosthetic capsular device and an IOL that couples to the one or more tabs, notches, or ribs on an interior surface of the device. FIG. 40 illustrates an example injector cartridge for use in a method of inserting a prosthetic intraocular device and/or an intraocular lens according to some embodiments herein.

Thus, some embodiments herein are directed to a kit for use in an ophthalmic surgical procedure, the kit comprising: a prosthetic implant device for insertion into a natural capsular bag of an eye after removal of a cataract, and an IOL. In some embodiments, the prosthetic implant device may comprise one or more tabs, notches, or ribs on an interior surface of the device, which are configured to couple, contact, join, or otherwise interact with one or more notches of the IOL or of haptics attached to the IOL.

FIG. 40 illustrates an example injector cartridge 4000 for use in a method of inserting a prosthetic intraocular device and/or an intraocular lens according to some embodiments herein. In some embodiments, during ocular surgical procedures, injector cartridges may be used to inject devices and/or IOLs into the eye. Existing injectors comprise single-use or reusable cartridges that facilitate insertion of an ophthalmic prosthetic device or IOL through a corneal incision of an eye. However, existing injectors have several drawbacks, including an inability to insert a prosthetic capsular device followed by an IOL, without removing the injector from the corneal insertion. For example, using existing injectors, a prosthetic capsular device may be inserted into the eye after removal of a cataract. After this insertion, the injector must be removed from the corneal incision to reset the injector, the IOL must be inserted into the injector, and the injector must be reloaded and replaced into the incision. This procedure is time-consuming, inefficient, and may introduce additional risk of surgical complications, specifically the stretching of the corneal incision, in a patient's eye.

The injector cartridge 4000 of FIG. 40 may be configured to insert both a prosthetic capsular device and an IOL into an eye without removal of the device from a corneal incision. In some embodiments, a single injector cartridge and push rod system is provided that allows both the prosthetic capsular device and the IOL to be implanted in one insertion. The left side chamber 4002 may contain the capsule, and the right-side chamber 4004 may contain the IOL. For example, as illustrated, the injector may comprise two or more loading chambers and/or channels, each of which funnels into a single distal injection chamber 4006. In some embodiments, the injector may comprise a “double-barrel” with two loading chambers, one for a prosthetic capsular device and one for an IOL, which will further simplify and expedite implantation. In some embodiments, one or more gates 4008 are provided, which prevent the prosthetic capsular device or IOL from being pressed backward into the opposing loading chamber. In some embodiments, push rods, such as screw push rods 4010, may be used to selectively load the prosthetic capsular device or IOL into the single distal injection chamber. In some embodiments, a foam-tip sponge 4012 trails the prosthetic capsular device and IOL within the injector to ensure that the prosthetic capsular device and IOL properly enter the eye.

In some embodiments, a surgical method is provided for implanting an ophthalmic prosthetic device and/or an IOL into an eye, the method comprising: inserting, using an injection system, the ophthalmic prosthetic device and IOL, through a corneal incision of an eye. In some embodiments, the method comprises using a single injector cartridge and push rod system that allows both the prosthetic capsular device and the IOL to be implanted in one insertion. In some embodiments, the method comprises using an injector, which comprises a “double-barrel” configuration with two loading chambers, one for a prosthetic capsular device and one for an IOL.

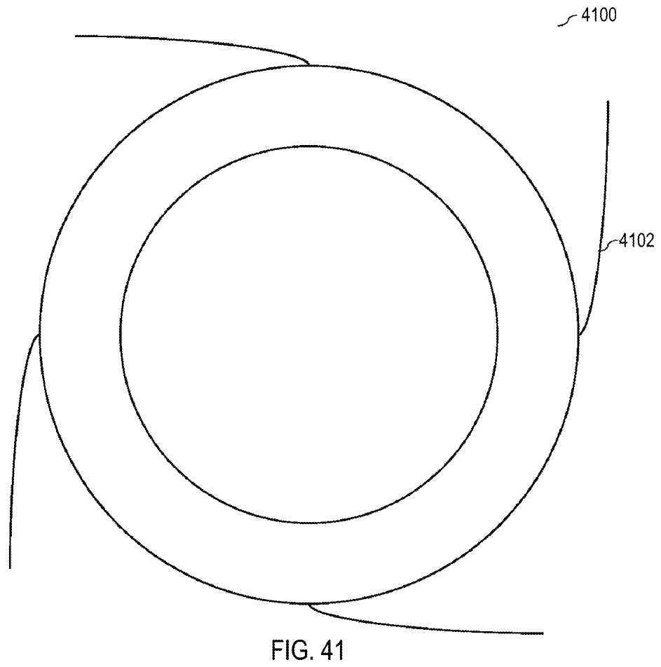

FIG. 41 illustrates another example prosthetic device according to some embodiments herein. In some embodiments, the prosthetic device 4100 may comprise one or more haptics 4102 extending from the exterior sidewall of the device 4100. In some embodiments, one or more haptics 4102 facilitate fixation of the device on the sclera and can be implanted in a patient with trauma or surgical complications that caused loss of natural capsular support. In some embodiments, the haptics 4102 may prevent rotation of the prosthetic capsular device to ensure centration of the device within the capsular bag, especially for asymmetric natural capsular bags.

In some embodiments, the prosthetic devices described herein may support any IOLs known to those skilled in the art, wherein the IOL may be coupled or inserted into the device within the eye. Existing devices only allow specific compatible IOLs to be fixated to the sclera of the eye. However, using the prosthetic capsular device of FIG. 41, any IOL could be coupled or inserted into the device, which fixates to the sclera and serves as a platform for the IOL. In some embodiments, the haptics and the prosthetic capsular device may provide scleral fixation, creating an artificial replacement lens capsule, in which various IOLs may be coupled or inserted.

In addition, the prosthetic capsular devices described herein may be tinted to prevent positive dysphotopsias, as well as cover iris defects from trauma or laser peripheral iridotomies for angle closure glaucoma.

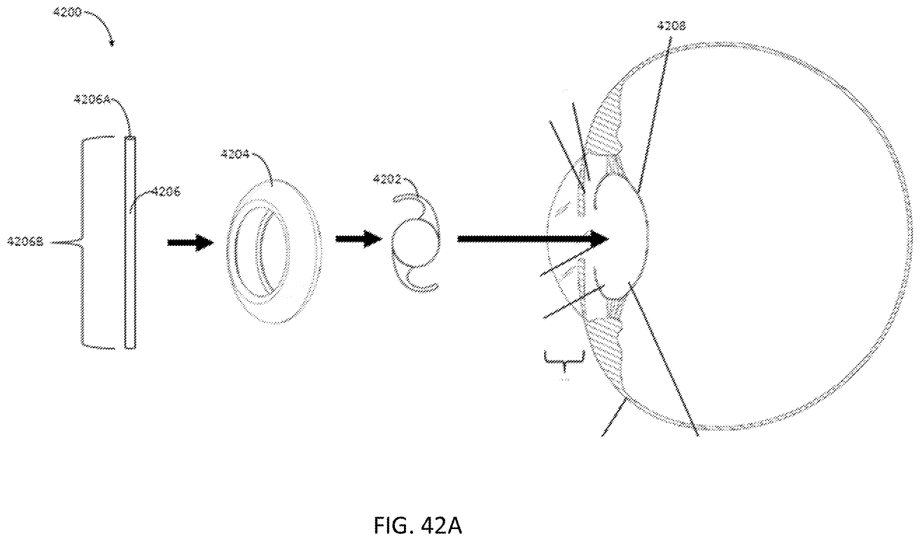

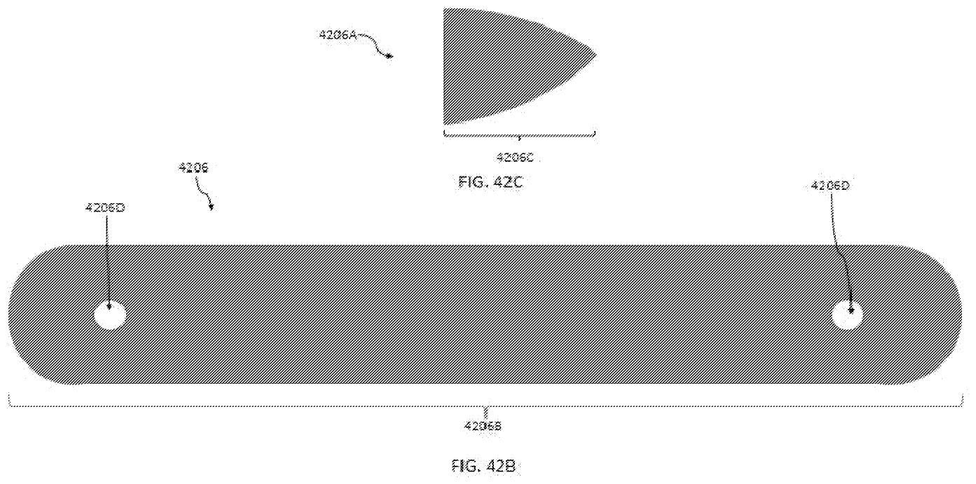

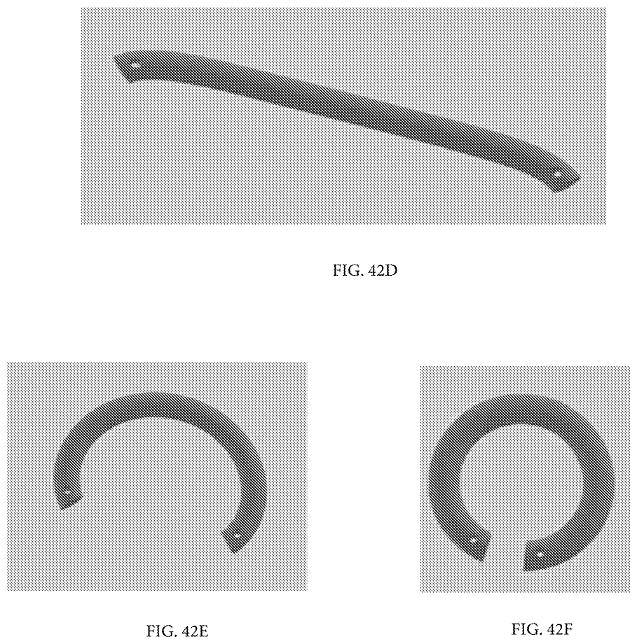

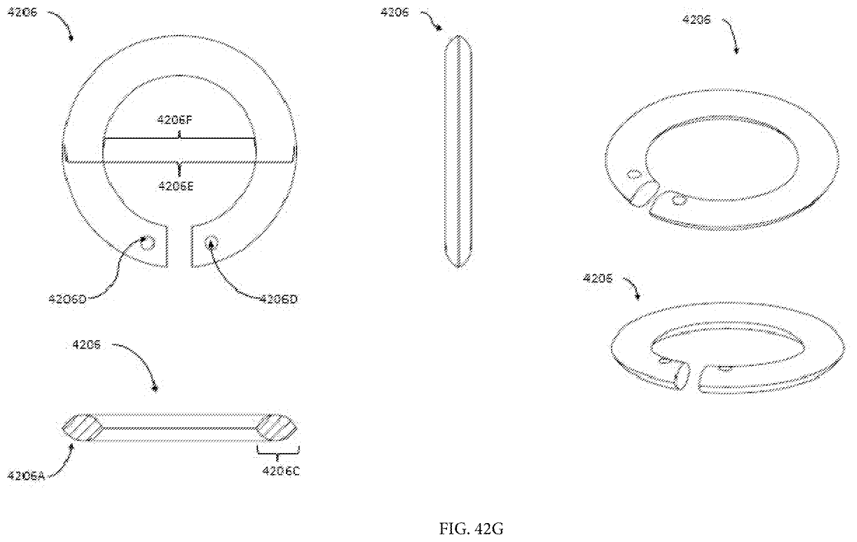

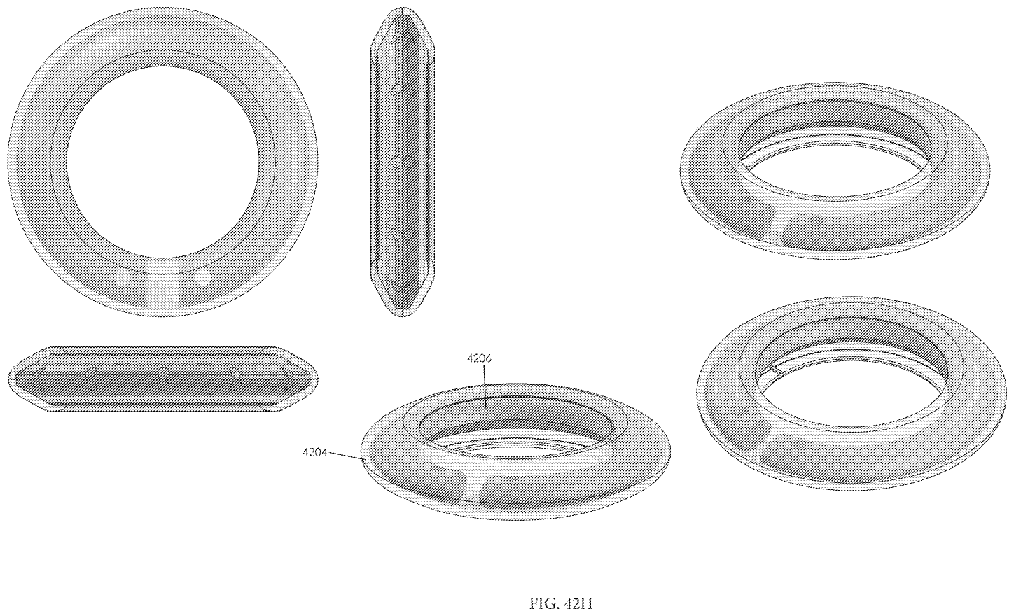

FIG. 42A is a schematic of a kit 4200 for use in an ophthalmic surgical procedure. The kit 4200 may comprise an IOL 4202, a prosthetic device 4204, and/or a drug delivery device 4206 or drug delivery implant. The IOL 4202 may an IOL as described in U.S. Pat. Nos. 8,900,300, 9,414,907, 9,358,103, 10,603,162, and/or any IOLs known to those skilled in the art. The prosthetic device 4202 may comprise any prosthetic device as described herein with reference to FIGS. 1-41.

FIGS. 42B-42H are schematics of the drug delivery device 4206. The drug delivery device 4206 may comprise a profile 4206A and a length 4206B. The profile 4206A may be a shape of the drug delivery device 4206 when viewed from an end or a sectional view of the drug delivery device 4206, as shown in FIGS. 42C and 42G. In some embodiments, the profile 4206A can be a circle, a square, a rectangle, an oval, a triangle, a rounded triangle, a pentagon, a trapezoid, an ellipse, a semicircle, a hexagon, an octagon, a ring, a rhombus, a star shape, and/or any other shape.

In some embodiments, the drug delivery device 4206 may be long and thin such that the length 4206B of the drug delivery device 4206 may be significantly larger than a width 4206C of the profile 4206A of the drug delivery device 4206. For example, the drug delivery device 4206 may be shaped as a pool noodle. In some embodiments, the length 4206B can be a 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 40 times, 45 times, 50 times, 60 times, 70 times, 80 times, 90 times, 100 times, 150 times, 200 times the width of the profile 4206B, and/or any value in between the aforementioned values. In some embodiments, the length 4206C of the drug delivery device 4206 can be about 1 mm, about 2 mm, about 3 mm, about 4 mm, about 5 mm, about 6 mm, about 7 mm, about 8 mm, about 9 mm, about 10 mm, about 15 mm, about 20 mm, about 25 mm, about 30 mm, about 35 mm, about 40 mm, about 50 mm, and/or any values between the aforementioned values. In some embodiments, the length 4206C of the drug delivery device 4206 can be about 28.5 mm.

The long and thin shape of the drug delivery device 4206 may allow the drug delivery device 4206 to flex or bend along the length 4206B of the drug delivery device 4206. In some embodiments, the drug delivery device 4206 may flex or bend from a straight configuration to a ring or discontinuous ring configuration, as shown in FIGS. 42D-42F, to fit inside an interior cavity of prosthetic device 4204 so the drug delivery device 4206 may contact an interior surface or sidewall of the prosthetic device 4204, as shown in FIG. 42H. In some embodiments, the drug delivery device 4206 may comprise a bias to be substantially straight, however, when the drug delivery device 4206 is positioned in the prosthetic device 4204, the drug delivery device 4206 may take on the shape of the prosthetic device 4204, for example, a ring like shape, or substantially curved configuration, or substantially circumferential configuration. In some embodiments, the drug delivery device 4206 can comprise a bias to be a ring like shape, or substantially curved configuration, or substantially circumferential configuration; however, when the drug delivery device 4206 is inserted and positioned through a delivery cannula, trochart, or injector, the drug delivery device 4206 can take on a substantially straight or linear configuration in order to be inserted through the delivery cannula, trochart, or injector, and into the prosthetic device 4204. In some embodiments, the drug delivery 4206 may form an arc such that the drug delivery device 4206 forms a portion of the ring or discontinuous circle shape.

In some embodiments, an outer diameter 4206E of the drug delivery device 4206 when the drug delivery device 4206 is in the ring configuration or discontinuous ring configuration may be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm, about 8.2 mm, about 8.4 mm, about 8.6 mm, about 8.8 mm, about 9.0 mm, about 9.2 mm, about 9.4 mm, about 9.6 mm, about 9.8 mm, about 10.0 mm, about 10.5 mm, about 11.0 mm, about 11.5 mm, about 12.0 mm and/or within a range defined by two of the aforementioned values. In some embodiments, the outer diameter 4206F may be about 9.15 mm.

In some embodiments, an inner diameter 4206F of the drug delivery device 4206 when the drug delivery device 4206 is in the ring configuration or discontinuous ring configuration may be about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, about 5.0 mm, about 5.2 mm, about 5.4 mm, about 5.6 mm, about 5.8 mm, about 6.0 mm, about 6.2 mm, about 6.4 mm, about 6.6 mm, about 6.8 mm, about 7.0 mm, about 7.2 mm, about 7.4 mm, about 7.6 mm, about 7.8 mm, about 8.0 mm and/or within a range defined by two of the aforementioned values. In some embodiments, the inner diameter 4206F may be about 6.0 mm.

In some embodiments, the length 4206B of the drug delivery device 4206 may be equal to or about a circumference of the interior cavity of the prosthetic device 4204 wherein the circumference of the interior cavity is the circumference of the prosthetic device 4204 where opposite sides of the interior surface of the prosthetic device 4204 are furthest apart, or where the inner cavity of the prosthetic device 4204 has a largest diameter. In some embodiments, the length 4206B of the drug delivery device 4206 may be greater than the circumference of the interior cavity of the prosthetic device 4204 such that at least a portion of the drug delivery device 4206 overlaps when the drug delivery device 4206 is in the inner cavity of the prosthetic device 4204.

In some embodiments, the drug delivery device 4206 may be shaped and/or sized such that when the drug delivery device 4206 is in the interior cavity of the prosthetic device 4204 none of the drug delivery device 4206 blocks or overlaps an opening of the prosthetic device 4204. In some embodiments, the drug delivery device 4206 may be shaped and/or sized such that when the drug delivery device 4206 is in the interior cavity of the prosthetic device 4204 a portion of the drug delivery device 4206 blocks or overlaps the opening of the prosthetic device 4204. In some embodiments, the drug delivery device 4206 can block or overlap a diameter of the opening of the prosthetic implant device 4204 by about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm and/or any value in between the aforementioned values.

In some embodiments, the length of the drug delivery device 4206 may be less than the circumference of the interior cavity of the prosthetic device 4204. In these embodiments, one drug delivery device 4206 may be inserted into the interior cavity of the prosthetic device 4204, or two or more drug delivery devices 4206 may be inserted into the interior cavity of the prosthetic device. In some embodiments, a sum of the lengths 4206B of the two or more drug delivery devices 4206 may be equal to or greater than a circumference of the interior cavity of the prosthetic device 4204 so the two or more drug delivery devices 4206 may form the entire ring or circular shape. In some embodiments, the sum of the lengths 4206B of the two or more drug delivery devices 4206 may be less than the circumference of the interior cavity of the prosthetic device so the two or more drug delivery devices 4206 form an arc or a portion of the ring or circular shape.

In some embodiments, the drug delivery device 4206 may comprise one or more bio-erodible materials. The bio-erodible material may comprise collagen, alginates, poly(lactic-glycolic acid) (PLGA), polyanhydrides, poly caprolactone, poly(trimethylene carbonate), polyphosphazenes, and/or any other bio-erodible materials. In some embodiments, the drug delivery device 4206 may not be bio-erodible. In some embodiments, the drug delivery device 4206 may comprise silicone, vulcanized silicone, and/or a silicone polymer.

In some embodiments, the one or more bio-erodible materials may be impregnated or infused with one or more drugs. In some embodiments, the drug delivery device 4206 may be immersed in a solution containing the one or more drugs. In these embodiments, the one or more drugs may be dissolved in a solvent that may swell silicone. In some embodiments, the drug delivery device 4206 may be immersed in the solvent with the dissolved one or more drugs. In some embodiments, the drug delivery device 4206 may be removed from the solvent with the dissolved one or more drugs after a predetermined immersion period. In some embodiments, after the drug delivery device 4206 is removed from the solvent, the solvent may evaporate, leaving the one or more drugs impregnated or infused within the drug delivery device 4206. In some embodiments, the drug delivery device 4206 may have a volume of and/or hold a volume of the one or more drugs of about 0.01 ml, about 0.02 ml, about 0.03 ml, about 0.04 ml, about 0.05 ml, about 0.06 ml, about 0.07 ml, about 0.08 ml, about 0.09 ml, 0.10 ml, about 0.11 ml, about 0.12 ml, about 0.13 ml, about 0.14 ml, about 0.15 ml, about 0.16 ml, about 0.17 ml, about 0.18 ml, about 0.19 ml, about 0.20 ml, and/or any value in between the aforementioned values. In some embodiments, a volume of the drug delivery device 4206 and/or the one or more drugs may be about 87.37 mm³.

In some embodiments, the drug delivery device 4206 may erode over a period of time. As the drug delivery device 4206 erodes, the drug delivery device 4206 can slowly release the one or more drugs in a sustained manner into the eye over the period of time. In some embodiments, the drug delivery device 4206 may include one or more drug release holes, eyelets, slits, and/or openings that allow a drug inside the drug delivery device 4206 to flow from inside the drug delivery device 4206 to the exterior of the drug delivery device 4206.

In some embodiments, the drug delivery device 4206 may include anti-inflammatory drugs (NSAIDs, Steroids), antifibrotics (Mitomycin), immune modulators (Cyclosporin, Tacrolimus, methotrexate), Anti-viral (Gancyclovir, Valcyclovir, acyclovir), Antibiotics, Antifungal (Voriconazol, Natamycin, Amphoteracin B), Anti-Vegf drugs (Avastin, Lucentis, Eylea) and/or any other drugs approved for intraocularly delivery and external delivery through eye drops.

In some embodiments, the drug delivery device 4206 may include one or more holes, eyelets, slits, and/or openings 4206D. In some embodiments, the one or more holes, eyelets, slits, and/or openings 4206D can allow the drug release device 4206 to be inserted into the prosthetic device 4204 with an injector. In some embodiments, the injector can be an injector as described herein with reference to FIG. 40. In some embodiments, the injector can be an injector as described in U.S. Pat. Nos. 8,900,300, 9,414,907, 9,358,103, 10,603,162, and/or any injectors known to those skilled in the art. In some embodiments, the injector may include a plunger pushrod. In some embodiments, the injector may include a screw advance pushrod system. In some embodiments, the drug delivery device 4206 can be shaped like a capsular tension ring.

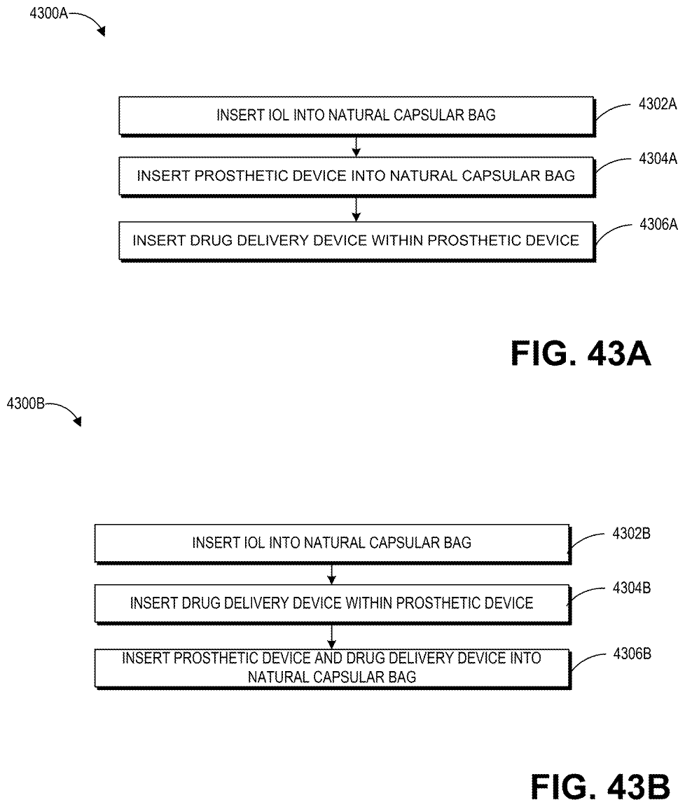

In some embodiments, as describe further below with reference to FIGS. 43A and 43B, the drug delivery device 4206 may be coupled to and/or bound to the inner surface or sidewall of the prosthetic device 4204 before the prosthetic device 4204 is inserted into a natural capsular bag, or the drug delivery device 4204 may be inserted into the inner cavity of the prosthetic device 4204 after the prosthetic device 4204 is inserted into a natural capsular bag.

FIG. 43A is a flow chart of method 4300A for inserting kit 4200 into the natural capsular bag. In some embodiments, the IOL 4202 may be inserted or implanted into the natural capsular bag at step 4302A of method 4300A. In some embodiments, before the IOL 4202 may be inserted into the natural capsular bag at step 4302A, an incision may be made in the cornea. In some embodiments, the IOL 4202 may be inserted according to any of the methods disclosed herein and/or methods described in U.S. Pat. Nos. 8,900,300, 9,414,907, 9,358,103, and 10,603,162. In some embodiments, the IOL 4202 may be inserted into the natural capsular bag such that haptics of the IOL 4202 are located or positioned outside of the natural capsular bag.

In some embodiments, at step 4304A the prosthetic device 4204 may be inserted into the natural capsular bag. In some embodiments, the prosthetic device 4024 may be inserted according to any of the methods disclosed herein and/or methods described in U.S. Pat. Nos. 8,900,300, 9,414,907, 9,358,103, and 10,603,162. In some embodiments, the prosthetic device 4204 may be inserted into the natural capsular bag such that the IOL 4202 is posterior to a posterior opening of the prosthetic device 4204. In some embodiments, the prosthetic device 4204 may be inserted into the natural capsular bag such that the IOL 4202 sits within the posterior opening of the prosthetic device 4204. In some embodiments, the prosthetic device 4204 may be inserted into the natural capsular bag such that the IOL 4202 is coupled to the posterior opening of the prosthetic device 4204.

In some embodiments, at step 4306A the drug delivery device 4206 may be inserted into the inner cavity of the prosthetic device 4204. In some embodiments, multiple drug delivery devices 4206 may be inserted into the prosthetic device 4204 at step 4306A. In some embodiments, the drug delivery device 4206 may be inserted into the inner cavity of the prosthetic device 4204. In some embodiments, the drug delivery device 4206 may be inserted into the prosthetic device 4204 via an injector as described above with reference to FIGS. 42A-42E. In some embodiments, the drug delivery device 4206 may be in the straight configuration when the drug delivery device 4206 is in the injector. In some embodiments, when the drug delivery device 4206 is inserted into the prosthetic device 4204, the drug delivery device 4206 may automatically flex or bend from the straight configuration to the ring or discontinuous ring configuration.

In some embodiments, after the drug delivery device 4206 is inserted into the prosthetic device 4204 at step 4306A, the incision in the cornea may be stitched or closed according to any of the methods disclosed herein and/or methods described in U.S. Pat. Nos. 8,900,300, 9,414,907, 9,358,103, and 10,603,162.

In embodiments where the drug delivery device 4206 is bio-erodible or biodegradable, after the drug delivery device 4206 is partially eroded or fully eroded such that a portion of the drugs of the drug delivery device 4206 are released or all of the drugs of the drug delivery device 4206 are released, step 4306A of method 4300A may be repeated such that at least a second drug delivery device 4206 can be inserted into the prosthetic device 4204.

In embodiments where the drug delivery device 4206 is not bio-erodible or biodegradable, after a portion of the drugs of the drug delivery device 4206 are released or all of the drugs of the drug delivery device 4206 are released, the drug delivery device 4206 may be removed from the interior of the prosthetic device 4206. In some embodiments, step 4306A of method 4300A may be repeated such that at least a second drug delivery device 4206 may be inserted into the prosthetic device 4204.

FIG. 43B is a flow chart of method 4300B for inserting kit 4200 into the natural capsular bag. In some embodiments, the IOL 4202 may be inserted or implanted into the natural capsular bag at step 4302B of method 4300B. In some embodiments, before the IOL 4202 may be inserted into the natural capsular bag at step 4302B, an incision may be made in the cornea. In some embodiments, the IOL 4202 may be inserted according to any of the methods disclosed herein and/or methods described in U.S. Pat. Nos. 8,900,300, 9,414,907, 9,358,103, and 10,603,162. In some embodiments, the IOL 4202 may be inserted into the natural capsular bag such that haptics of the IOL 4202 are located or positioned outside of the natural capsular bag.

In some embodiments, at step 4304B, the drug delivery device 4206 can be inserted into the inner cavity of the prosthetic device 4204. In some embodiments, step 4304B of method 4300B can be completed before the IOL 4202 is inserted or implanted into the natural capsular bag at step 4302B, or step 4304B of method 4300B can be completed after the IOL 4202 is inserted or implanted into the natural capsular bag at step 4302B. In some embodiments, the drug delivery device 4206 can be inserted into the inner cavity of the prosthetic device 4204 via an injector as described above with reference to FIGS. 42A-42H. In some embodiments, the drug delivery device 4206 may be in the straight configuration when the drug delivery device 4206 is in the injector. In some embodiments, when the drug delivery device 4206 is inserted into the prosthetic device 4204, the drug delivery device 4206 may automatically flex or bend from the straight configuration to the ring or discontinuous ring configuration. In some embodiments, the drug delivery device 4206 may be coupled to an inner surface of the prosthetic device 4204. In some embodiments, the drug delivery device 4206 may be a portion of the prosthetic device 4204 such that the drug delivery device 4206 is not removable from the prosthetic device 4206.

In some embodiments, at step 4306B of method 4300B, the prosthetic device 4204 and the drug delivery device 4206 may be inserted into the natural capsular bag. In some embodiments, the prosthetic device 4204 and the drug delivery device 4206 may be inserted according to any of the methods disclosed herein and/or methods described in U.S. Pat. Nos. 8,900,300, 9,414,907, 9,358,103, and 10,603,162. In some embodiments, the prosthetic device 4204 and the drug delivery device 4206 may be inserted into the natural capsular bag such that the IOL 4202 is posterior to a posterior opening of the prosthetic device 4204. In some embodiments, the prosthetic device 4204 and the drug delivery device 4206 may be inserted into the natural capsular bag such that the IOL 4202 sits within the posterior opening of the prosthetic device 4204. In some embodiments, the prosthetic device 4204 and the drug delivery device 4206 may be inserted into the natural capsular bag such that the IOL 4202 is coupled to the posterior opening of the prosthetic device 4204. In some embodiments, one or more IOL 4202 may be placed in front of and/or behind drug delivery device 4204. In some embodiments, drug delivery device 4204 may be configured to secure one or more IOL 4202 within or adjacent to the housing structure of prosthetic device 4204.

In embodiments where the drug delivery device 4206 is bio-erodible or biodegradable, after the drug delivery device 4206 is partially eroded or fully eroded such that a portion of the drugs of the drug delivery device 4206 are released or all of the drugs of the drug delivery device 4206 are released, at least a second drug delivery device 4206 can be inserted into the inner volume of the prosthetic device 4204.

In embodiments where the drug delivery device 4206 is not bio-erodible or biodegradable, after a portion of the drugs of the drug delivery device 4206 are released or all of the drugs of the drug delivery device 4206 are released, the drug delivery device 4206 may be removed from the inner volume of the prosthetic device 4204. In some embodiments, step 4306A of method 4300A may be repeated such that at least a second drug delivery device 4206 may be inserted into the prosthetic device 4204.

In some embodiments, after a portion of the drug of the drug delivery device 4206 are released or all of the drug of the drug delivery device 4206 are released, the prosthetic device 4204 and the drug delivery device 4206 may be removed from the natural capsular bag. In some embodiments, at least a second prosthetic device 4204 and/or at least a second drug delivery device 4206 can be inserted into the natural capsular bag according to methods 4300A, 4300B as described herein.

It is to be appreciated that although the drug delivery device 4206 is described herein as being inserted into the inner volume of the prosthetic device 4206, the drug delivery device may be coupled anywhere on or in the prosthetic device 4206, or anywhere in the natural capsular bag relative to the prosthetic device 4206 without deviating from the scope of this disclosure.

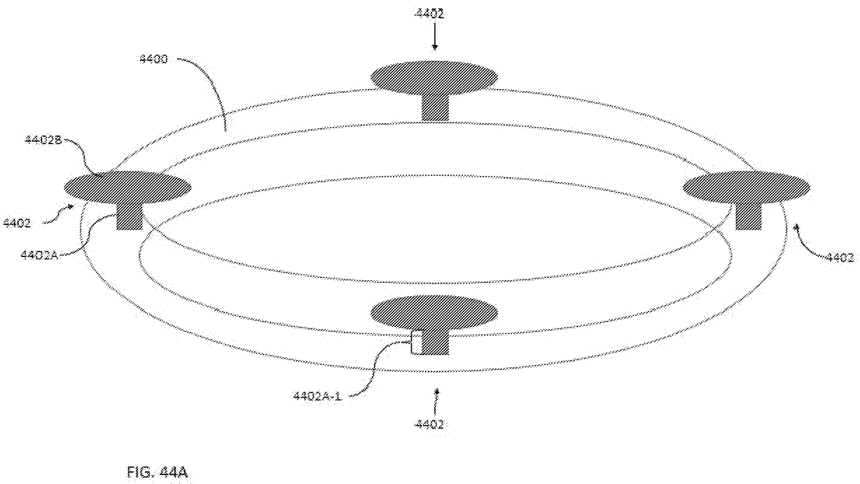

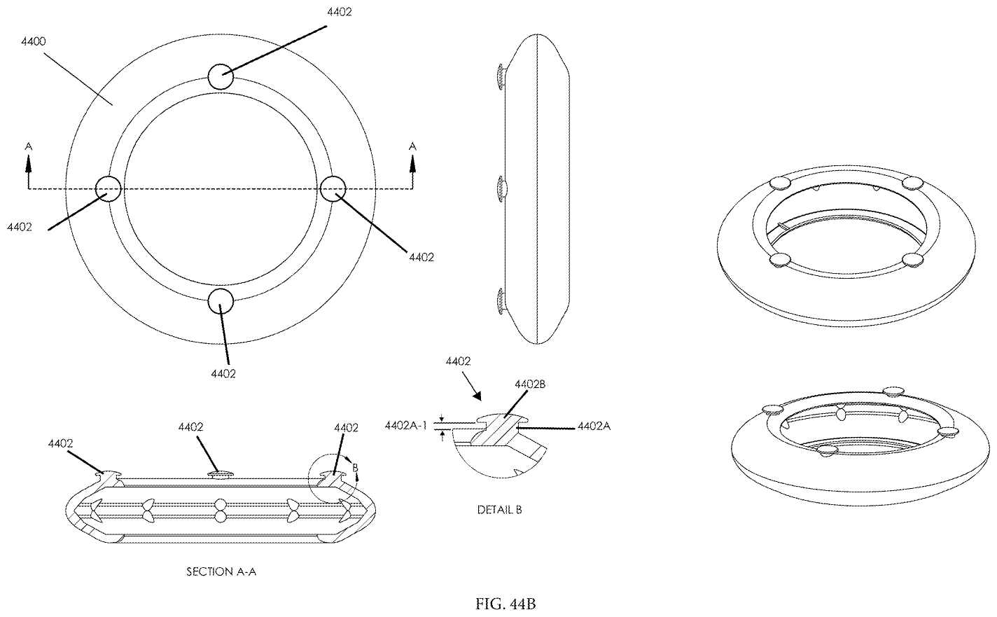

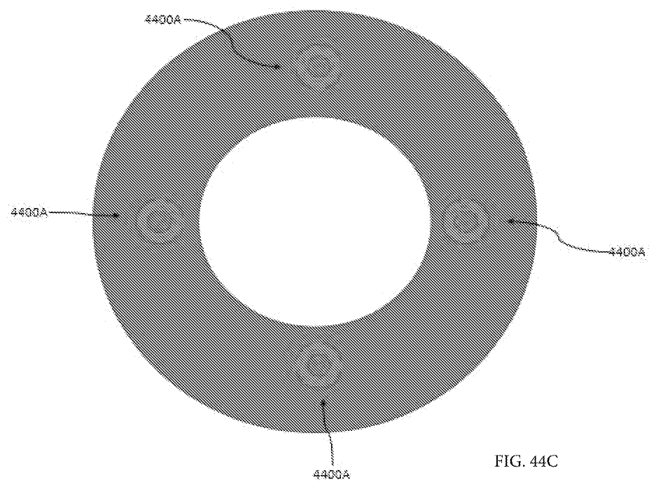

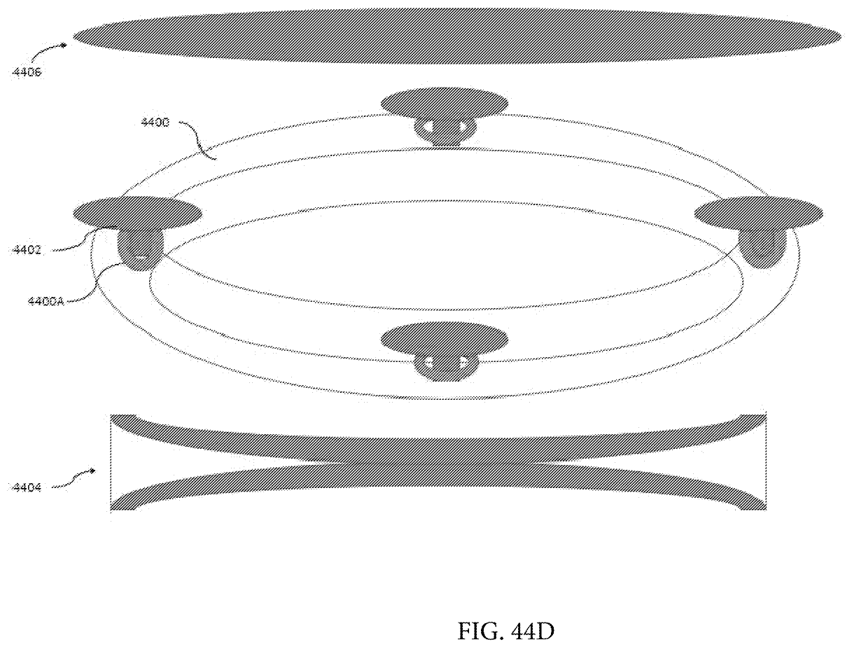

FIGS. 44A-44D are schematics of another prosthetic capsular device 4400 according to some embodiments herein. In some embodiments, the prosthetic capsular device 4400 may include one or more fixation members 4402. In some embodiments, the one or more fixation members 4402 may be transcapsular. In some embodiments, the prosthetic capsular device 4400 may include four fixation members 4402. In some embodiments, the prosthetic capsular device 4400 may include between two and eight fixation members 4402. In some embodiments, the one or more fixation member 4402 may be equally circumferentially spaced on the prosthetic capsular device 4400. In some embodiments, the one or more fixation members 4402 may include a stalk portion 4402A and/or a securement portion 4402B. In some embodiments, a first end of the stalk portion 4402A may be coupled to an outer surface of the prosthetic capsular device 4400. In some embodiments, the securement portion 4402B may be coupled to a second end of the stalk portion 4402A.

In some embodiments, the stalk portion 4402A may be cylindrical or any other prismatic shape. In some embodiments, the stalk portion 4402A may include a height 4402A-1. In some embodiments, the height 4402A-1 of the stalk portion 4402A may be about the thickness of an anterior capsule. In some embodiments, the height 4402A-1 of the stalk portion 4402A may be greater than the thickness of the anterior capsule.

In some embodiments, the stalk portion 4402A may include a diameter or width. In some embodiments, the diameter or width of the stalk portion 4402A may be about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, and/or any value in between the aforementioned values.

In some embodiments, the securement portion 4402B may be a portion of material with a circular, hemispherical, rectangular, triangular, and/or any other shaped profile, wherein the profile is the shape of the securement portion 4402B when view from the top of the securement portion 4402B. The top of the securement portion 4402B may be an end of the securement portion 4402B opposite the end of the securement portion 4402B coupled to the second end of the stalk portion 4402A. In some embodiments, the securement portion 4402B may be shaped such that the securement portion 4402B is a shape of a mushroom tip and/or a curved bulb.

In some embodiments, the profile of the securement portion 4402B can include a diameter or width. In some embodiments, the diameter or width of the profile of the securement portion 4402B can be greater than the diameter or width of the stalk portion 4402A. In some embodiments, the diameter or width of the profile of the securement portion 4402B may be about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm and/or any value in between the aforementioned values.

In some embodiments, the one or more fixation members 4402 may be coupled to the prosthetic capsular device 4400 at a radial distance from a center of the prosthetic capsular device 4400. In some embodiments, the radial distance may be about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, about 4.0 mm, and/or any value between the aforementioned values.

In some embodiments, one or more microrhexises 4400A may be formed in the anterior capsule. In some embodiments, one or more microrhexises 4400A may be formed in the periphery of the anterior capsule. In some embodiments, one or more microrhexises 4400A may be formed by a femtosecond laser. In some embodiments, the one or more microrhexises 4400A may be located at the radial distance from the center of the anterior capsule such that the one or more microrhexises 4400A may be substantially aligned with the one or more fixation members 4402. In some embodiments, the radial distance may be about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, about 4.0 mm, and/or any value between the aforementioned values. In some embodiments, four microrhexises 4400A may be formed in the anterior capsule. In some embodiments, between two and eight microrhexises 4400A may be formed in the anterior capsule.

In some embodiments, the microrhexises 4400A may include a diameter or width. In some embodiments, the diameter or width of the microrhexises 4400A may be the diameter or width of the stalk portion 4204A of the one or more fixation members 4402. In some embodiments, the diameter or width of the microrhexises 4400A may be about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, and/or any value in between the aforementioned values. In some embodiments, the diameter or width of the microrhexises 4400A may be less than the diameter or width of the profile of the securement portion 4402B.

In some embodiments, the prosthetic capsular device 4400 may be inserted into the natural capsular bag such that the one or more fixation members 4402 may be anterior to the rest of the prosthetic device 4400. In some embodiments, the one or more fixation members 4402 may be inserted through the one or more microrhexises 4400A such that the securement portion 4402B of the one or more fixation member 4402 extend into the sulcus. In some embodiments, the securement portion 4402B may be shaped such that the securement portion 4402B may be inserted through the one or more microrhexises 4400A and the securement portion 4402B may not be removed through the one or more microhexises 4400A. In some embodiments, the securement portion 4402B may secure the prosthetic device 4400 to the anterior capsule. In some embodiments, the securement portion 4402B may create a transcapsular bond. In some embodiments, the securement portion 4402B may prevent defixation of the prosthetic device 4400 to the anterior capsule.

In some embodiments, the one or more fixation members 4402 may couple or fix the prosthetic capsular device 4400 to the zonular complex. In some embodiments, when the ciliary muscle contracts, expands, and/or relaxes, the ciliary muscle may move the one or more fixation members 4402 in the radial direction. In some embodiments, the movement of the one or more fixation members 4402 in the radial direction may contract or expand the prosthetic capsular device 4400 in the radial direction.

In some embodiments, the contraction or expansion of the prosthetic capsular device 4400 in the radial direction may cause the prosthetic capsular device 4400 to expand or contract in the anterior/posterior direction. In some embodiments, the prosthetic capsular device 4400 may expand in the anterior/posterior direction when the ciliary muscle is contracted. In some embodiments, the prosthetic device 4400 may contract in the anterior/posterior direction when the ciliary muscle is relaxed.

In some embodiments, the expansion and/or contraction of the prosthetic capsular device 4400 in the anterior/posterior direction may allow the prosthetic capsular device 4400 to be a spring between a posterior lens 4404 and/or an anterior lens 4406, as shown in FIG. 44C. In some embodiments, movement of the ciliary muscle may change a distance between the posterior lens 4404 and an anterior surface of the prosthetic capsular device 4400. In some embodiments, movement of the ciliary muscle may change a distance between the anterior lens 4406 and a posterior surface of the prosthetic capsular device 4400. In some embodiments, movement of the ciliary muscle may change a distance between the posterior lens 4400 and the anterior lens 4406.

In some embodiments, the posterior lens 4404 may be coupled to the prosthetic capsular device 4400. In some embodiments, the posterior lens 4404 may be posterior to the prosthetic capsular device 4400 in the natural capsular bag. In some embodiments, the anterior lens 4406 may be coupled to the prosthetic capsular device 4400. In some embodiments, the anterior lens 4406 may be coupled to an anterior opening of the prosthetic device 4400, in an inner cavity of the prosthetic capsular device 4400, and/or in the ciliary sulcus.

In some embodiments, the posterior lens 4404 and/or the anterior lens 4406 may be a minus powered lens (i.e., plano concave, biconcave, or concave plano). In some embodiments, the posterior lens 4404 and/or the anterior lens 4406 may be a plus powered lens (i.e., plano convex, biconvex, or convex plano. In some embodiments, the posterior lens 4404 may be a minus powered lens, and the anterior lens 4406 may be a plus powered lens.

In some embodiments, the posterior lens 4404 and/or the anterior lens 4406 may include light adjustable power. The light adjustable power may adjust a refractive state of the eye after the prosthetic capsular device 4400, the posterior lens 4404, and/or the anterior lens 4406 are inserted into an eye. In some embodiments, the light adjustable power may include one or more sensors that detect an amount of light and/or contraction or relaxation of the ciliary muscle. In some embodiments, the light adjustable power may electro-mechanically, via an algorithm, adjust or change a power of the posterior lens 4404 and/or the anterior lens 4406 (e.g., change lens curvature, change lens thickness, change lens refractive index).

### ADDITIONAL EMBODIMENTS

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than restrictive sense.

Indeed, although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in several ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

What is claimed is:

1. A prosthetic capsular device configured to be inserted in a natural capsular bag of an eye, the prosthetic capsular device comprising:
a housing structure comprising:
an anterior portion comprising:
an anterior circular opening;
an anterior rim surrounding the anterior circular opening and defining a perimeter of the anterior circular opening; and
an anterior sidewall connected to the anterior rim and extending laterally outward and posteriorly from the anterior rim to a center plane of the housing structure;
a posterior portion comprising:
a posterior circular opening;
a posterior rim surrounding the posterior circular opening and defining a perimeter of the posterior circular opening; and
a posterior sidewall connected to the posterior rim and extending laterally outward and anteriorly from the posterior rim to the longitudinal center plane of the housing structure;
an interior cavity formed between the anterior circular opening and the posterior circular opening, the interior cavity configured to house a drug delivery device; and
a groove formed by one or more ribs, the one or more ribs formed along a circumference of the interior cavity at the longitudinal center plane of the housing structure, wherein each rib of the one or more ribs comprises a top surface and a bottom surface formed a rib angle, and wherein the groove is configured to hold the drug delivery device in place within the interior cavity of the housing structure; and
a drug delivery device having a ring or an arc configuration, wherein the drug delivery device is held in place within the groove formed by the one or more ribs within the interior cavity of the housing structure.

2. The prosthetic capsular device of claim 1, wherein the drug delivery device, when viewed in profile, comprises a circle, a square, a rectangle, an oval, a triangle, a rounded triangle, a pentagon, a trapezoid, an ellipse, a semicircle, a hexagon, an octagon, a ring, a rhombus, or a star shape.

3. The prosthetic capsular device of claim 1, wherein the drug delivery device comprises a length and a width, and wherein the ration of the length to the width is between about 2:1 and about 200:1.

4. The prosthetic capsular device of claim 3, wherein the length is between 1 mm and 50 mm.

5. The prosthetic capsular device of claim 1, wherein the drug delivery device contacts an interior surface or an interior sidewall of the housing structure.

6. The prosthetic capsular device of claim 1, wherein the drug delivery device is configured to flex or bend from a straight configuration to the ring or the arc configuration.

7. The prosthetic capsular device of claim 1, wherein the drug delivery device comprises an outer diameter between about 3 mm and about 12 mm.

8. The prosthetic capsular device of claim 1, wherein a length of the drug delivery device is substantially equal to a circumference of the interior cavity of the housing structure.

9. The prosthetic capsular device of claim 1, wherein a length of the drug delivery device is greater than a circumference of the interior cavity of the housing structure, such that at least a portion of the drug delivery device overlaps when the drug delivery device is held in the interior cavity.

10. The prosthetic capsular device of claim 1, wherein a length of the drug delivery device is less than a circumference of the interior cavity of the housing structure.

11. The prosthetic capsular device of claim 10, further comprising a second drug delivery device having an arc configuration, wherein the second drug delivery device is held in place within the groove formed by the one or more ribs within the interior cavity of the housing structure.

12. The prosthetic capsular device of claim 11, wherein the second drug delivery device comprises a second length, wherein a sum of the length of the drug delivery device and the second length of the second drug delivery device is about equal to the circumference of the interior cavity of the housing structure.

13. The prosthetic capsular device of claim 12, wherein the drug delivery device and the second drug delivery device substantially form an entire ring shape within the groove of the housing structure.

14. The prosthetic capsular device of claim 1, wherein the drug delivery device comprises a bio-erodible material.

15. The prosthetic capsular device of claim 14, wherein the bio-erodible material comprises collagen, alginates, poly(lactic-glycolic acid) (PLGA), polyanhydrides, poly caprolactone, poly(trimethylene carbonate), or polyphosphazenes.

16. The prosthetic capsular device of claim 14, wherein the bio-erodible material is impregnated or infused with one or more drugs.

17. The prosthetic capsular device of claim 16, wherein the one or more drugs comprise anti-inflammatory drugs (NSAIDs, Steroids), antifibrotics (Mitomycin), immune modulators (Cyclosporin, Tacrolimus, methotrexate), Anti-viral (Gancyclovir, Valcyclovir, acyclovir), Antibiotics, Antifungal (Voriconazol, Natamycin, Amphoteracin B), or Anti-Vegf drugs (Avastin, Lucentis, Eylea).

18. The prosthetic capsular device of claim 1, wherein the drug delivery device comprises silicone, vulcanized silicone, and/or a silicone polymer.

19. The prosthetic capsular device of claim 1, wherein the drug delivery device comprises one or more holes, eyelets, slits, and/or openings.

20. The prosthetic capsular device of claim 19, wherein the one or more holes, eyelets, slits, and/or openings are configured to allow the drug delivery device to be placed within the interior cavity of the housing structure with an injector.
